(19) United States
(12) Patent Application Publication
(10) Pub. No.: US 2024/0254082 A1
(43) Pub. Date: Aug. 1, 2024

(54) Substituted Isoindoline-1, 3-diketone PDE4 Inhibitor and Pharmaceutical Application Thereof

(71) Applicant: SUZHOU INTRAGRAND PHARMA CO., LTD, Suzhou, Jiangsu (CN)

(72) Inventors: Jiawang ZHU, Suzhou, Jiangsu (CN); Yao YAO, Suzhou, Jiangsu (CN)

(73) Assignee: SUZHOU INTRAGRAND PHARMA CO., LTD, Suzhou, Jiangsu (CN)

(21) Appl. No.: 18/293,761

(22) PCT Filed: Apr. 2, 2022
(86) PCT No.: PCT/CN2022/088776

§ 371 Date: Jan. 30, 2024

(30) Foreign Application Priority Data
Aug. 13, 2021 (CN) 202110932229.9

(51) Int. Cl. C07D 209/48; A61K 31/4035; A61P 29/00

(52) U.S. Cl. CPC C07D 209/48; A61K 31/4035; A61P 29/00

(57) ABSTRACT

The present disclosure relates to a compound as shown in formula I and its racemates, stereoisomers, tantomers, isotopic labels, solvates, polymorphs, esters, prodrugs or pharmaceutically acceptable salts as well as a pharmaceutical composition containing same, and preparation methods and medical uses thereof. The structure of formula I is as follows:

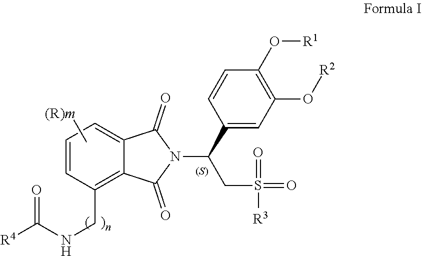

### TECHNICAL FIELD

The present disclosure belongs to the field of medicine, and in particular relates to a substituted isoindoline-1, 3-diketone PDE4 inhibitor as well as a preparation method and pharmaceutical application thereof.

### BACKGROUND

Cyclic adenosine monophosphate (cAMP) as a second messenger plays a fairly significant role in biological processes. Studies have found that the deficiency or inactivation of the cyclic adenosine monophosphate has contributed to diseases such as asthma, pulmonary obstructive disease, and inflammation (Lowe and Cheng, Drugs of the Future, 17(9): 799-807, 1992), while an increase in cAMP level of inflammatory leukocytes inhibits the release of inflammatory mediators including TNF-α and NF-κB; and furthermore, the increase in the cAMP level will also lead to the relaxation of airway smooth muscle (ASM).

The main biological mechanism for inactivation of cyclic adenosine monophosphate is due to the destruction of cAMP level by cyclic nucleotide phosphodiesterase (PDE) family (Beavo and Reitsnyder, Trends in Pharm., 11: 150-155, 1990). It is known that there are currently 11 family enzymes of PDE members, and inhibition of type 4 PDE (type IV PDE) has a significant effect on the promotion of cAMP and the release of inflammatory mediators (Verghes et al., Journal of Pharmacology and Experimental Therapeutics, 272(3): 1313-1320, 1995). Thus, the organic compounds that selectively inhibiting of PDE4 has the potential to inhibit airway inflammation, promote the relaxation of ASM, and treat skin inflammation.

Inhibition of the PDE4 enzyme may block the activity or production of certain cytokines including alpha-tumor necrosis factor (TNF-α). Alpha-tumor necrosis factor is a cytokine that is released primarily by mononuclear phagocytes in response to immune stimuli. TNF-α is capable of promoting most cellular processes such as differentiation, recruitment, proliferation and protein degradation. At low levels, TNF-α has a protective effect against infectious agents, tumors and tissue damage, but TNF-α plays an inducing and exacerbating role in many diseases. When administered to mammals or humans, TNF-α causes or exacerbates inflammation, fever, cardiovascular effects, hemorrhage, and acute reactions similar to those occurring during acute infection and shock.

Arthritis, arthritic conditions (such as osteoarthritis and rheumatoid arthritis), enteritis (such as segmental ileitis and ulcerative colitis), sepsis, psoriasis, atopic dermatitis (AD), contact dermatitis and chronic obstructive pulmonary disease (COPD), chronic pneumonia, acute respiratory distress syndrome (ARDS), vitiligo, prurigo nodularis, vulvodynia, fibrotic disease, cachexia, autoimmune disease, ankylosing spondylitis, osteoporosis, segmental ileitis, ulcerative colitis, enteritis, multiple sclerosis (MS), discoid lupus erythematosus, systemic lupus erythematosus, radiation injury, hyperoxia alveolar damage (Tracey et al., 1987, Nature, 330: 662-664 and Hinshaw et al., 1990, Circle. Shock, 30: 279-292 (endotoxin shock); Millar et al., 1989, Lancet, 2: 712-714 and Ferrai-Baliviera et al., 1989, Arch Surg., 124: 1400-1405 (Adult Respiratory Distress Syndrome); Bertolini et al., 1986, Nature, 319: 516-518; Pignet et al., 1990, Nature, 344: 245-247, Bissonnette et al., 1989, Inflammation, 13: 329-339, and Baughman et al., 1990, J Lab. Lin. Med., 115: 36-42 (Chronic Pneumonia); Elliot et al., 1995, Int. J. Pharmac., 17: 141-145 (Rheumatoid Arthritis); Von Dullemen et al., 1995, Gastroenterology, 109: 129-135 (Segmental Ileitis)) and other inflammatory diseases are common intractable diseases, and in these inflammatory reactions, alpha-tumor necrosis factor plays a crucial role. The inhibition of the alpha-tumor necrosis factor can be seen to show an effective blockage effect on chronic and acute inflammatory reactions in animal models of inflammatory diseases. A number of small-molecule inhibitors have been found to be effective in inflammatory diseases involving alpha-tumor necrosis factor (see Review by Lowe, 1998, Exp. Opin. Ther. Patents, 8: 1309-1332). Such molecules are the substituted phenethyl sulfones described in U.S. Pat. Nos. 6,020,358, 6,962,940, as well as WO0134606A1, WO0025777, WO2012083153, WO2018157779A1, WO0134604, WO2012083153, and WO2016169533. Apremilast is disclosed in patent US2003187052, and Chinese patents of the same family are CN1652772, CN1965823, CN101683334, and CN03811093.8.

However, the biological activity of isoindoline-1, 3-diketone PDE4 inhibitors in the prior art is relatively general, and thus the efficacy thereof is not high. Therefore, it is necessary to provide an isoindoline-1, 3-diketone PDE4 inhibitor with a novel structure and good efficacy to treat many diseases.

### SUMMARY

In order to solve the problems existing in the prior art and improve the efficacy, the present disclosure provides a compound as shown in formula I and its racemates, stereoisomers, tautomers, isotopic labels, solvates, polymorphs, esters, prodrugs or pharmaceutically acceptable salts:

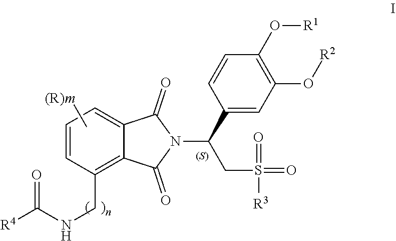

in the formula, each R may independently be H, deuterium, halogen, amino, hydroxyl, cyano, nitro, and the following groups unsubstituted or optionally substituted with one, two or more Ra: alkyl groups of C1-C16, heteroalkyl groups of C1-C16, cycloalkyl groups of C3-C12, R′SO₂NH—, alkyl- of R′SO₂NH-C1-C16, alkyl- of R′SO₂-C1-C16, R′SO₂—, 3-12 membered heterocyclic groups, aryl groups of C6-C14, and 5-14 membered heteroaryl groups; or cyclic moiety may be formed between two Rs at different positions independently.

Ra is independently selected from deuterium, halogen, amino, hydroxyl, cyano, nitro, oxo (═O), and the following groups unsubstituted or optionally substituted with one, two or more Rb: hydrocarbyl groups of C1-C16, heteroalkyl groups of C1-C16, cycloalkyl groups of C3-C12, 3-12 membered heterocyclic groups, aryl groups of C6-C14, and 5-14 membered heteroaryl groups;

R′ is independently selected from the following groups unsubstituted or optionally substituted with one, two or more Rb: hydrocarbyl groups of C1-C16, heteroalkyl groups of C1-C16, cycloalkyl groups of C3-C12, 3-12 membered heterocyclic groups, aryl groups of C6-C14, and 5-14 membered heteroaryl groups;

Rb is selected from deuterium, halogen, amino, hydroxyl, cyano, nitro, oxo (=O), as well as hydrocarbyl groups of C1-C16, heteroalkyl groups of C1-C16, cycloalkyl groups of C3-C12, 3-12 membered heterocyclic groups, aryl groups of C6-C14, and 5-14 membered heteroaryl groups; m is 1, 2, or 3; n is 0 or 1;

R¹ refers to the following groups unsubstituted or optionally substituted with one, two or more R1a: hydrocarbyl groups of C1-C16, heteroalkyl groups of C1-C16, and cycloalkyl groups of C3-C12;

R² refers to the following groups unsubstituted or optionally substituted with one, two or more R2a: hydrocarbyl groups of C1-C16, heteroalkyl groups of C1-C16, and cycloalkyl groups of C3-C12;

alternatively, R¹ and R² may form cyclic moiety;

R³ refers to the following groups unsubstituted or optionally substituted with one, two or more R3a: hydrocarbyl groups of C1-C16, heteroalkyl groups of C1-C16, and cycloalkyl groups of C3-C12;

R⁴ refers to the following groups unsubstituted or optionally substituted with one, two or more R4a: hydrocarbyl groups of C1-C16, heteroalkyl groups of C1-C16, and cycloalkyl groups of C3-C12;

R1a is independently selected from deuterium, halogen, amino, hydroxyl, cyano, nitro, oxo (═O), and the following groups unsubstituted or optionally substituted with one, two or more R1b: hydrocarbyl groups of C1-C16, heteroalkyl groups of C1-C16, and cycloalkyl groups of C3-C12;

R2a is independently selected from deuterium, halogen, amino, hydroxyl, cyano, nitro, oxo (═O), and the following groups unsubstituted or optionally substituted with one, two or more R2b: hydrocarbyl groups of C1-C16, heteroalkyl groups of C1-C16, and cycloalkyl groups of C3-C12;

R3a is independently selected from deuterium, halogen, amino, hydroxyl, cyano, nitro, oxo (═O), and the following groups unsubstituted or optionally substituted with one, two or more R3b: hydrocarbyl groups of C1-C16, heteroalkyl groups of C1-C16, and cycloalkyl groups of C3-C12;

R4a is independently selected from deuterium, halogen, amino, hydroxyl, cyano, nitro, oxo (═O), and the following groups unsubstituted or optionally substituted with one, two or more R4b: hydrocarbyl groups of C1-C16, heteroalkyl groups of C1-C16, and cycloalkyl groups of C3-C12;

R1b is selected from deuterium, halogen, amino, hydroxyl, cyano, nitro, oxo (═O), as well as hydrocarbyl groups of C1-C16, and heteroalkyl groups of C1-C16;

R2b is selected from deuterium, halogen, amino, hydroxyl, cyano, nitro, oxo (═O), as well as hydrocarbyl groups of C1-C16, and heteroalkyl groups of C1-C16;

R3b is selected from deuterium, halogen, amino, hydroxyl, cyano, nitro, oxo (═O), as well as hydrocarbyl groups of C1-C16, and heteroalkyl groups of C1-C16; and

R4b is selected from deuterium, halogen, amino, hydroxyl, cyano, nitro, oxo (═O), as well as hydrocarbyl groups of C1-C16, and heteroalkyl groups of C1-C16.

According to some embodiments of the present disclosure, R¹ and R² can form 5-, 6-, and 7-membered cyclic miocty.

According to some embodiments of the present disclosure, the R may independently be a 5, 6, or 7 substituents.

According to some embodiments of the present disclosure, the hydrocarbyl groups of C1-C16 are alkyl groups of C1-C16, alkenyl groups of C2-C16, and alkynyl groups of C2-C16.

According to some embodiments of the present disclosure, each of the heteroalkyl groups of C1-C16 is an alkyl containing one, two or more heteroatoms selected from N, O, S; specifically, the heteroalkyl groups of C1-C16 may be selected from C1-C16 alkyloxy, C1-C8-alkyl OC1-C8 alkyl-, C1-C8-alkyl-O-C1-C8 alkyl-NH—, C1-C16 alkylthio-, C1-C8-alkyl-S-C1-C8 alkyl-, C1-C8-alkyl-S-C1-C8 alkyl-NH—, C1-C16 alkyl-NH—, C1-C8-alkyl-NH-C1-C8 alkyl-, NH₂-C1-C16 alkyl-, -C1-C8-alkyl-NH-C1-C8 alkyl-NH_{2;} and the number of carbon atoms in the heteroalkyl groups of C1-C16 may be further preferably C1-C12, more preferably C5-8.

According to some embodiments of the present disclosure, the two Rs together with the carbon atom to which they are attached form C5-6 membered cycloalkyl groups.

According to some embodiments of the present disclosure, the R is a 5, 6, or 7 substituent, and may independently be H, deuterium, halogen, amino, hydroxyl, cyano, nitro, and the following groups unsubstituted or optionally substituted with one, two or more Ra: alkyl groups of C1-C12, alkenyl groups of C2-C12, alkynyl groups of C2-C12, heteroalkyl groups of C1-C12, cycloalkyl groups of C3-C8, 3-10 membered heterocyclic groups, aryl groups of C6-C10, and 5-10 membered heteroaryl groups.

According to the exemplary embodiment of the present disclosure, the R is a 5, 6, or 7 substituent, and may independently be H, deuterium, halogen, amino, hydroxyl, cyano, nitro, and the following groups unsubstituted or optionally substituted with one, two or more Ra: alkyl groups of C5-C8, alkenyl groups of C5-C8, alkynyl groups of C5-C8, heteroalkyl groups of C5-C8, cycloalkyl groups of C3-C6, 3-6 membered heterocyclic groups, an aryl group of C6, and 5-6 membered heteroaryl groups.

According to some embodiments of the present disclosure, the Ra is independently selected from deuterium, halogen, amino, hydroxyl, cyano, nitro, oxo (═O), and the following groups unsubstituted or optionally substituted with one, two or more Rb: alkyl groups of C1-C12, alkenyl groups of C2-C12, alkynyl groups of C2-C12, heteroalkyl groups of C1-C12, cycloalkyl groups of C3-C8, 3-12 membered heterocyclic groups, aryl groups of C6-C14, and 5-10 membered heteroaryl groups.

According to the exemplary embodiment of the present disclosure, the Ra is independently selected from deuterium, halogen, amino, hydroxyl, cyano, nitro, oxo (═O), and the following groups unsubstituted or optionally substituted with one, two or more Rb: alkyl groups of C5-C8, alkenyl groups of C5-C8, alkynyl groups of C5-C8, heteroalkyl groups of C5-C8, cycloalkyl groups of C3-C6, 3-6 membered heterocyclic groups, an aryl group of C6, and 5-6 membered heteroaryl groups.

According to some embodiments of the present disclosure, R′ is independently selected from the following groups unsubstituted or optionally substituted with one, two or more Rb: alkyl groups of C1-C12, alkenyl groups of C2-C12, alkynyl groups of C2-C12, heteroalkyl groups of C1-C12, cycloalkyl groups of C3-C8, 3-12 membered heterocyclic groups, aryl groups of C6-C14, and 5-10 membered heteroaryl groups.

According to the exemplary embodiment of the present disclosure, the R′ is independently selected from deuterium, halogen, amino, hydroxyl, cyano, nitro, oxo (═O), and the following groups unsubstituted or optionally substituted with one, two or more Rb: alkyl groups of C5-C8, alkenyl groups of C5-C8, alkynyl groups of C5-C8, heteroalkyl groups of C5-C8, cycloalkyl groups of C3-C6, 3-6 membered heterocyclic groups, an aryl group of C6, and 5-6 membered heteroaryl groups.

According to some embodiments of the present disclosure, Rb is selected from deuterium, halogen, amino, hydroxyl, cyano, nitro, oxo (═O), as well as alkyl groups of C1-C12, alkenyl groups of C2-C12, alkynyl groups of C2-C12, heteroalkyl groups of C1-C12, cycloalkyl groups of C3-C8, 3-12 membered heterocyclic groups, aryl groups of C6-C14, and 5-10 membered heteroaryl groups.

According to the exemplary embodiment of the present disclosure, the Rb is selected from deuterium, halogen, amino, hydroxyl, cyano, nitro, oxo (═O), as well as alkyl groups of C5-C8, alkenyl groups of C5-C8, alkynyl groups of C5-C8, heteroalkyl groups of C5-C8, cycloalkyl groups of C3-C6, 3-6 membered heterocyclic groups, an aryl group of C6, and 5-6 membered heteroaryl groups.

According to some embodiments of the present disclosure,

R¹ refers to the following groups unsubstituted or optionally substituted with one, two or more R1a: alkyl groups of C1-C12, alkenyl groups of C2-C12, alkynyl groups of C2-C12, heteroalkyl groups of C1-C12, and cycloalkyl groups of C3-C8; the R1a is independently selected from deuterium, halogen, amino, hydroxyl, cyano, nitro, oxo (═O), and the following groups unsubstituted or optionally substituted with one, two or more R1b: alkyl groups of C1-C12, alkenyl groups of C2-C12, alkynyl groups of C2-C12, heteroalkyl groups of C1-C12, and cycloalkyl groups of C3-C12; the R1b is selected from deuterium, halogen, amino, hydroxyl, cyano, nitro, oxo (═O);

preferably, R¹ refers to the following groups unsubstituted or optionally substituted with one, two or more R1a: alkyl groups of C1-C6, alkenyl groups of C2-C6, alkynyl groups of C2-C6, heteroalkyl groups of C1-C6, and cycloalkyl groups of C3-C6; the R1a is independently selected from deuterium, halogen, amino, hydroxyl, cyano, nitro, oxo (═O), and the following groups unsubstituted or optionally substituted with one, two or more R1b: alkyl groups of C1-C6, alkenyl groups of C2-C6, alkynyl groups of C2-C6, heteroalkyl groups of C1-C6, and cycloalkyl groups of C3-C6; the R1b is selected from deuterium, halogen, amino, hydroxyl, cyano, nitro, oxo (═O);

more preferably, R¹ refers to the following groups unsubstituted or optionally substituted with one, two or more R1a: alkyl groups of C1-C3, alkenyl groups of C2-C3, alkynyl groups of C2-C3, heteroalkyl groups of C1-C3, and cycloalkyl groups of C3-C6; the R1a is independently selected from deuterium, halogen, amino, hydroxyl, cyano, nitro, oxo (═O), and the following groups unsubstituted or optionally substituted with one, two or more R1b: alkyl groups of C1-C3, alkenyl groups of C2-C3, alkynyl groups of C2-C3, heteroalkyl groups of C1-C3, and cycloalkyl groups of C3-C6; the R1b is selected from deuterium, halogen, amino, hydroxyl, cyano, nitro, oxo (═O);

R² refers to the following groups unsubstituted or optionally substituted with one, two or more R2a: alkyl groups of C1-C12, alkenyl groups of C2-C12, alkynyl groups of C2-C12, heteroalkyl groups of C1-C12, and cycloalkyl groups of C3-C8; the R2a is independently selected from deuterium, halogen, amino, hydroxyl, cyano, nitro, oxo (═O), and the following groups unsubstituted or optionally substituted with one, two or more R2b: alkyl groups of C1-C12, alkenyl groups of C2-C12, alkynyl groups of C2-C12, heteroalkyl groups of C1-C12, and cycloalkyl groups of C3-C12; the R2b is selected from deuterium, halogen, amino, hydroxyl, cyano, nitro, oxo (═O);

preferably, R² refers to the following groups unsubstituted or optionally substituted with one, two or more R2a: alkyl groups of C1-C6, alkenyl groups of C2-C6, alkynyl groups of C2-C6, heteroalkyl groups of C1-C6, and cycloalkyl groups of C3-C6; the R2a is independently selected from deuterium, halogen, amino, hydroxyl, cyano, nitro, oxo (═O), and the following groups unsubstituted or optionally substituted with one, two or more R2b: alkyl groups of C1-C6, alkenyl groups of C2-C6, alkynyl groups of C2-C6, heteroalkyl groups of C1-C6, and cycloalkyl groups of C3-C6; the R2b is selected from deuterium, halogen, amino, hydroxyl, cyano, nitro, oxo (═O);

more preferably, R² refers to the following groups unsubstituted or optionally substituted with one, two or more R2a: alkyl groups of C1-C5, alkenyl groups of C2-C5, alkynyl groups of C2-C5, heteroalkyl groups of C1-C5, and cycloalkyl groups of C3-C6; the R2a is independently selected from deuterium, halogen, amino, hydroxyl, cyano, nitro, oxo (═O), and the following groups unsubstituted or optionally substituted with one, two or more R2b: alkyl groups of C1-C5, alkenyl groups of C2-C5, alkynyl groups of C2-C5, heteroalkyl groups of C1-C5, and cycloalkyl groups of C3-C6; the R2b is selected from deuterium, halogen, amino, hydroxyl, cyano, nitro, oxo (═O);

R³ refers to the following groups unsubstituted or optionally substituted with one, two or more R3a: alkyl groups of C1-C12, alkenyl groups of C2-C12, alkynyl groups of C2-C12, heteroalkyl groups of C1-C12, and cycloalkyl groups of C3-C8; the R3a is independently selected from deuterium, halogen, amino, hydroxyl, cyano, nitro, oxo (═O), and the following groups unsubstituted or optionally substituted with one, two or more R3b: alkyl groups of C1-C12, alkenyl groups of C2-C12, alkynyl groups of C2-C12, heteroalkyl groups of C1-C12, and cycloalkyl groups of C3-C12; the R3b is selected from deuterium, halogen, amino, hydroxyl, cyano, nitro, oxo (═O);

preferably, R³ refers to the following groups unsubstituted or optionally substituted with one, two or more R3a: alkyl groups of C1-C6, alkenyl groups of C2-C6, alkynyl groups of C2-C6, heteroalkyl groups of C1-C6, and cycloalkyl groups of C3-C6; the R3a is independently selected from deuterium, halogen, amino, hydroxyl, cyano, nitro, oxo (═O), and the following groups unsubstituted or optionally substituted with one, two or more R3b: alkyl groups of C1-C6, alkenyl groups of C2-C6, alkynyl groups of C2-C6, heteroalkyl groups of C1-C6, and cycloalkyl groups of C3-C6; the R3b is selected from deuterium, halogen, amino, hydroxyl, cyano, nitro, oxo (═O);

more preferably, R³ refers to the following groups unsubstituted or optionally substituted with one, two or more R3a: alkyl groups of C1-C5, alkenyl groups of C2-C5, alkynyl groups of C2-C5, heteroalkyl groups of C1-C5, and cycloalkyl groups of C3-C6; the R3a is independently selected from deuterium, halogen, amino, hydroxyl, cyano, nitro, oxo (═O), and the following groups unsubstituted or optionally substituted with one, two or more R3b: alkyl groups of C1-C5, alkenyl groups of C2-C5, alkynyl groups of C2-C5, heteroalkyl groups of C1-C5, and cycloalkyl groups of C3-C6; the R3b is selected from deuterium, halogen, amino, hydroxyl, cyano, nitro, oxo (═O);

R⁴ refers to the following groups unsubstituted or optionally substituted with one, two or more R4a: alkyl groups of C1-C12, alkenyl groups of C2-C12, alkynyl groups of C2-C12, heteroalkyl groups of C1-C12, and cycloalkyl groups of C3-C8; the R4a is independently selected from deuterium, halogen, amino, hydroxyl, cyano, nitro, oxo (═O), and the following groups unsubstituted or optionally substituted with one, two or more R4b: alkyl groups of C1-C12, alkenyl groups of C2-C12, alkynyl groups of C2-C12, heteroalkyl groups of C1-C12, and cycloalkyl groups of C3-C12; the R4b is selected from deuterium, halogen, amino, hydroxyl, cyano, nitro, oxo (═O);

preferably, R⁴ refers to the following groups unsubstituted or optionally substituted with one, two or more R4a: alkyl groups of C1-C6, alkenyl groups of C2-C6, alkynyl groups of C2-C6, heteroalkyl groups of C1-C6, and cycloalkyl groups of C3-C6; the R4a is independently selected from deuterium, halogen, amino, hydroxyl, cyano, nitro, oxo (═O), and the following groups unsubstituted or optionally substituted with one, two or more R4b: alkyl groups of C1-C6, alkenyl groups of C2-C6, alkynyl groups of C2-C6, heteroalkyl groups of C1-C6, and cycloalkyl groups of C3-C6; and the R4b is selected from deuterium, halogen, amino, hydroxyl, cyano, nitro, oxo (═O).

According to the embodiments of the present disclosure, the compound as shown in formula I is further selected from the following formula II:

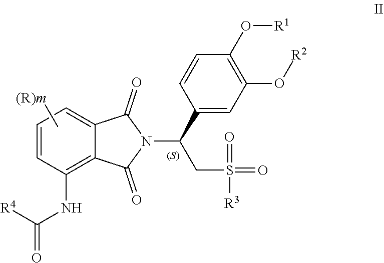

in formula II, the R, R¹, R², R³, R⁴, and m are as defined above.

According to the embodiments of the present disclosure, the compound as shown in formula I is further selected from the following formula III:

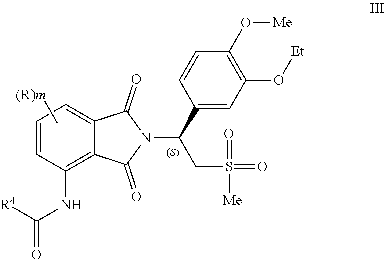

in formula III, the R, R⁴, and m are as defined above.

According to the embodiments of the present disclosure, the compound as shown in formula I is further selected from the following formula IV:

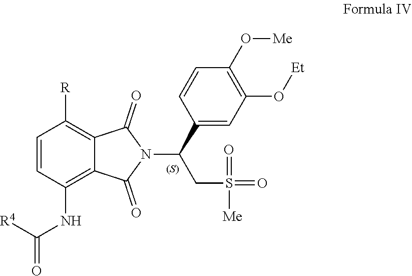

in formula IV, the R, m, and R⁴ are as defined above.

According to the embodiments of the present disclosure, in the compound as shown in formula I (including formula II-III) and its racemates, stereoisomers, tautomers, isotopic labels, solvates, polymorphs, esters, prodrugs or pharmaceutically acceptable salts, illustrative non-restrictive specific examples of the compound as shown in formula I are as follows:

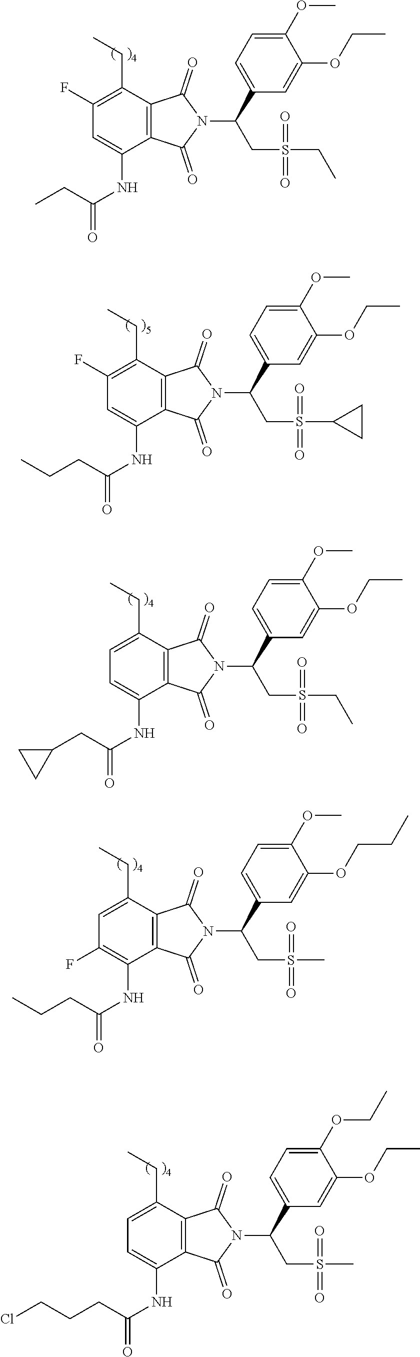

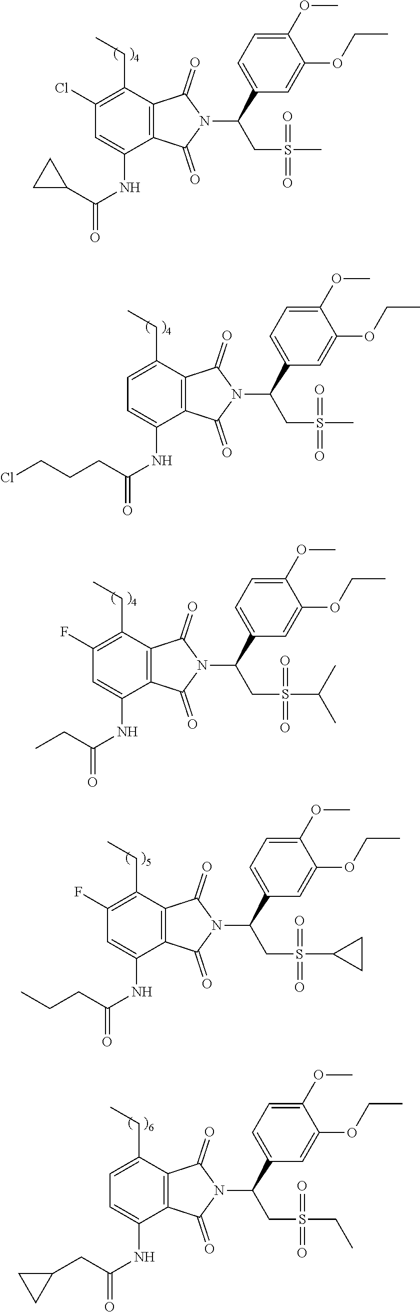

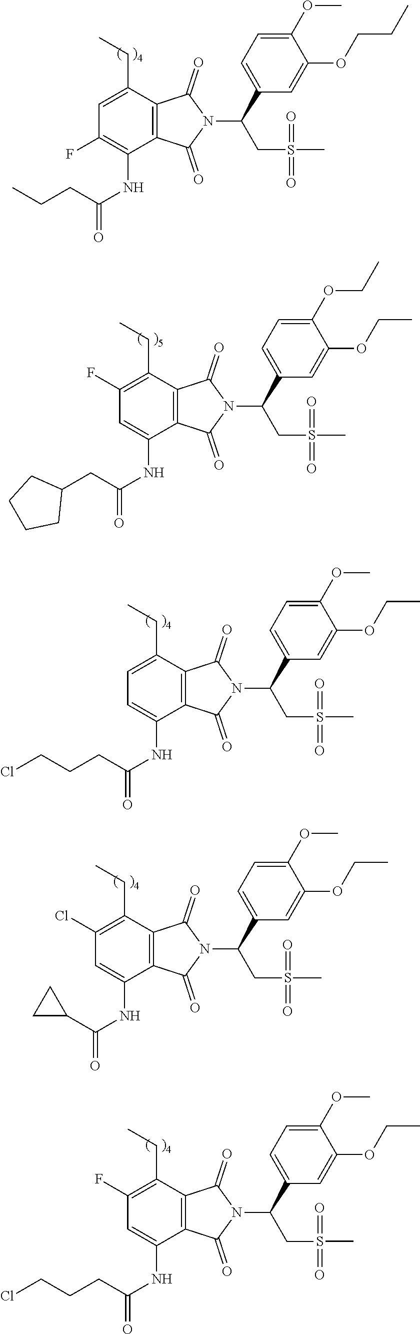

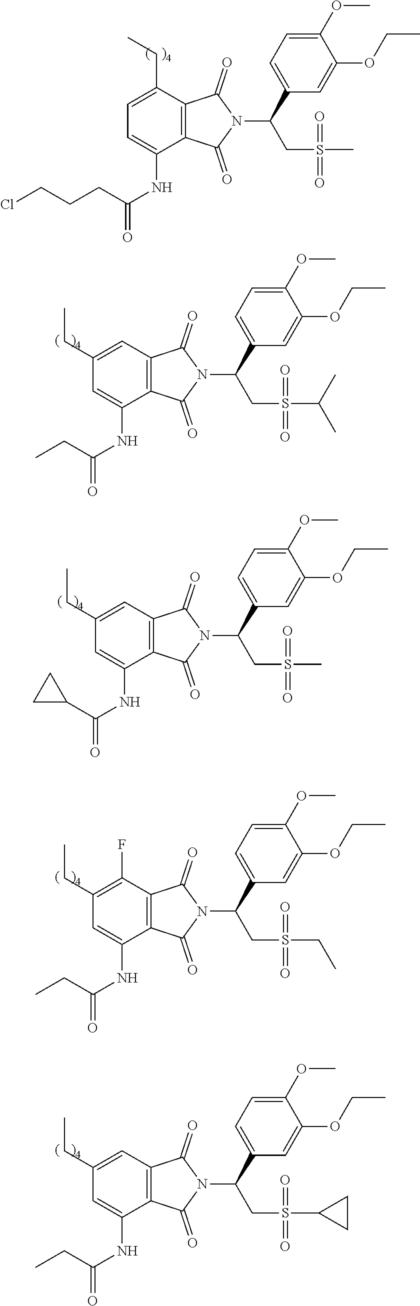

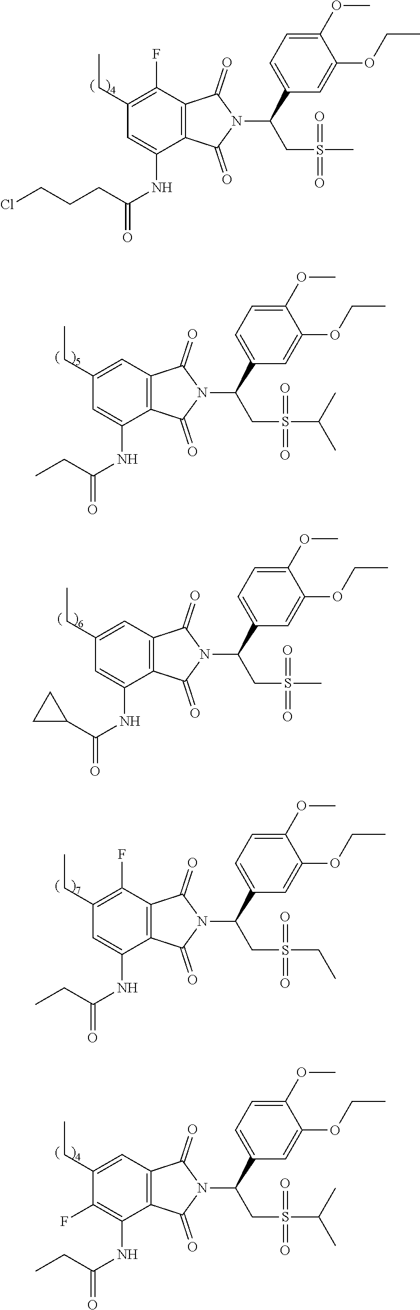

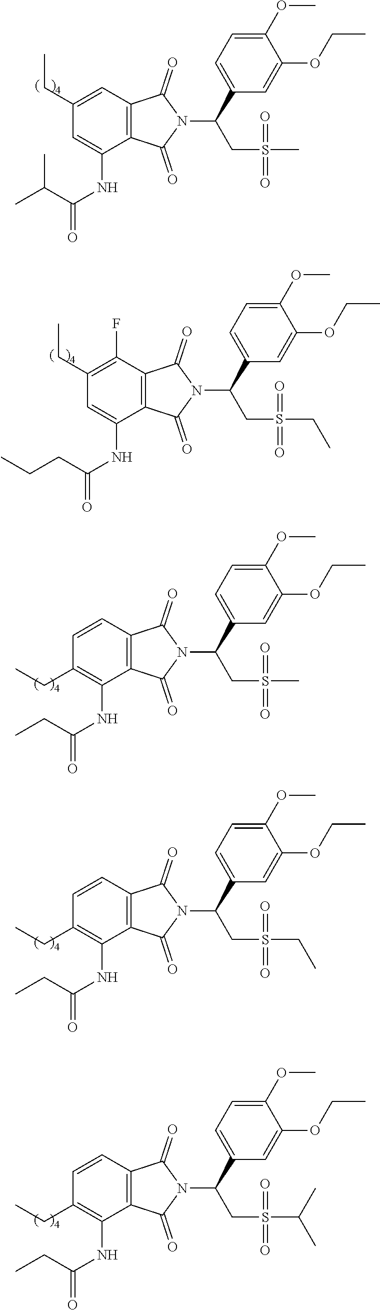

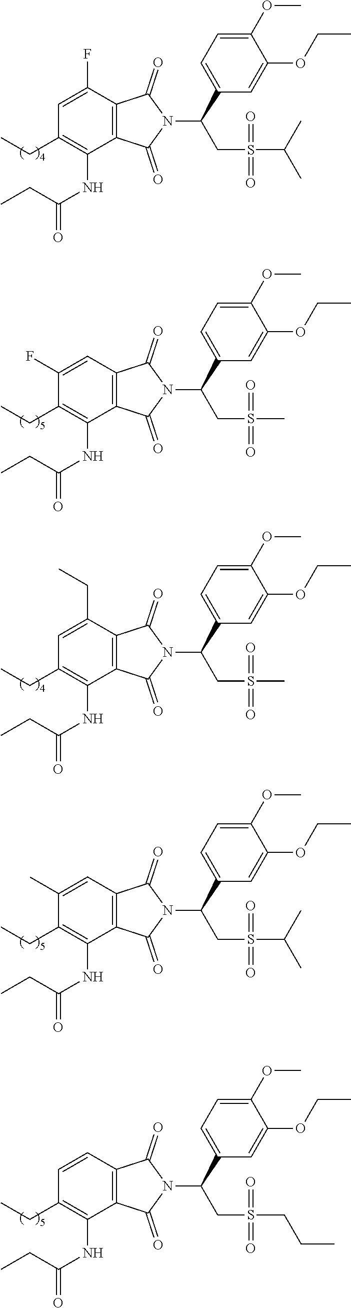

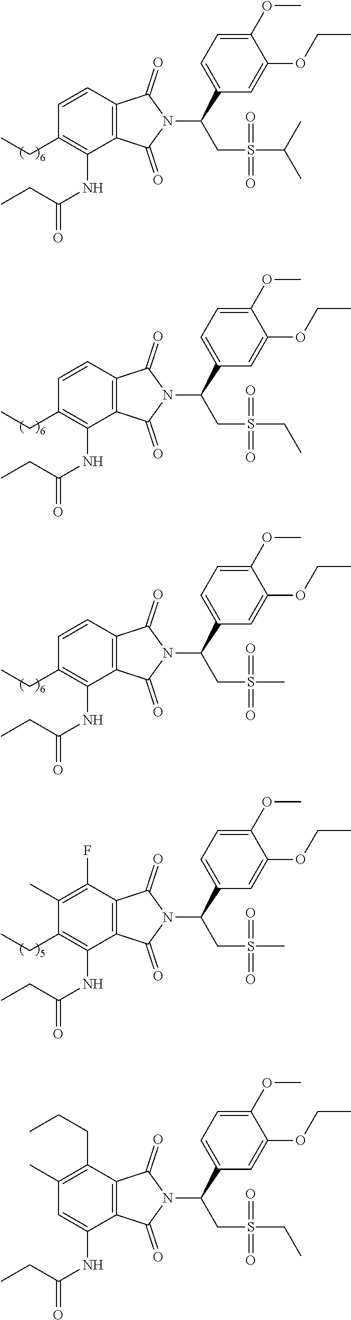

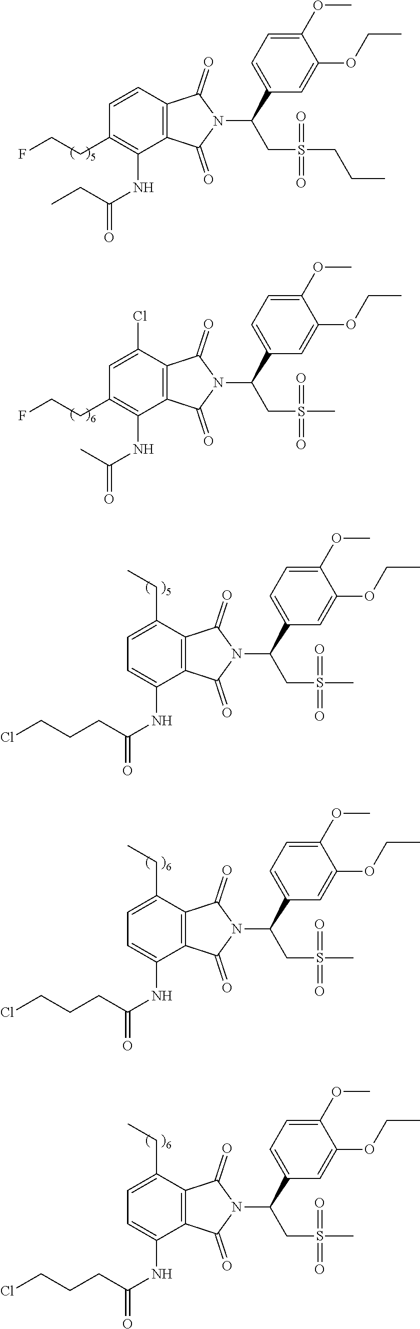

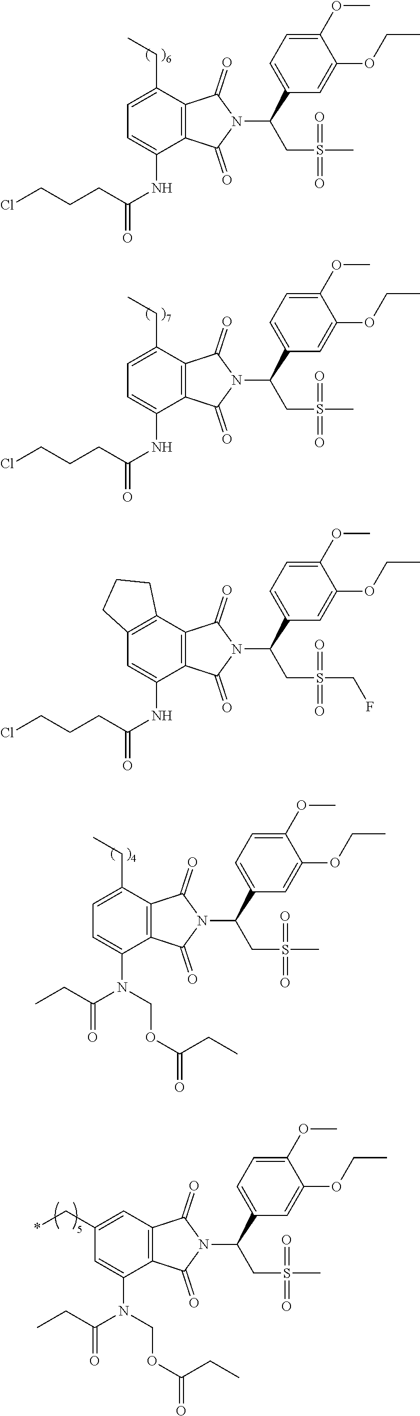

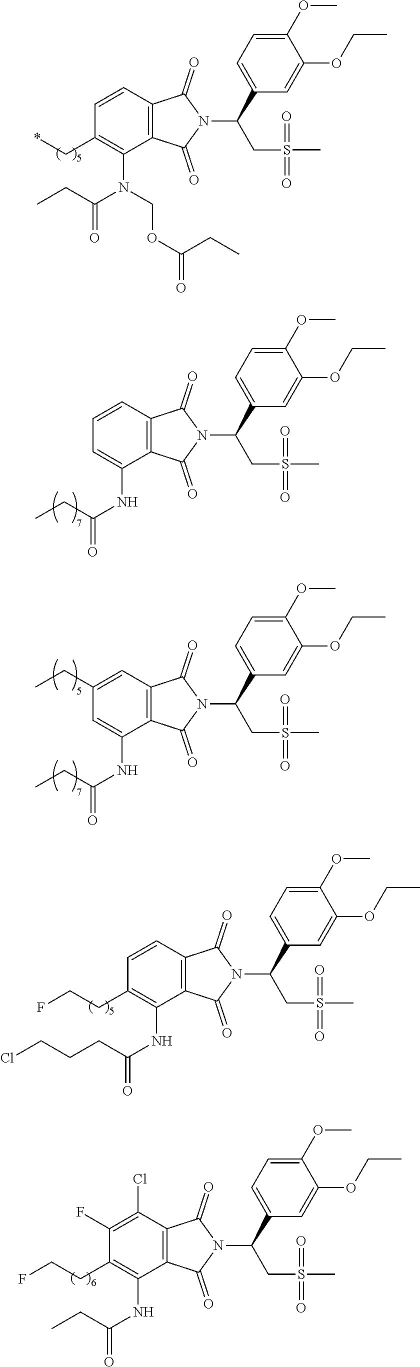

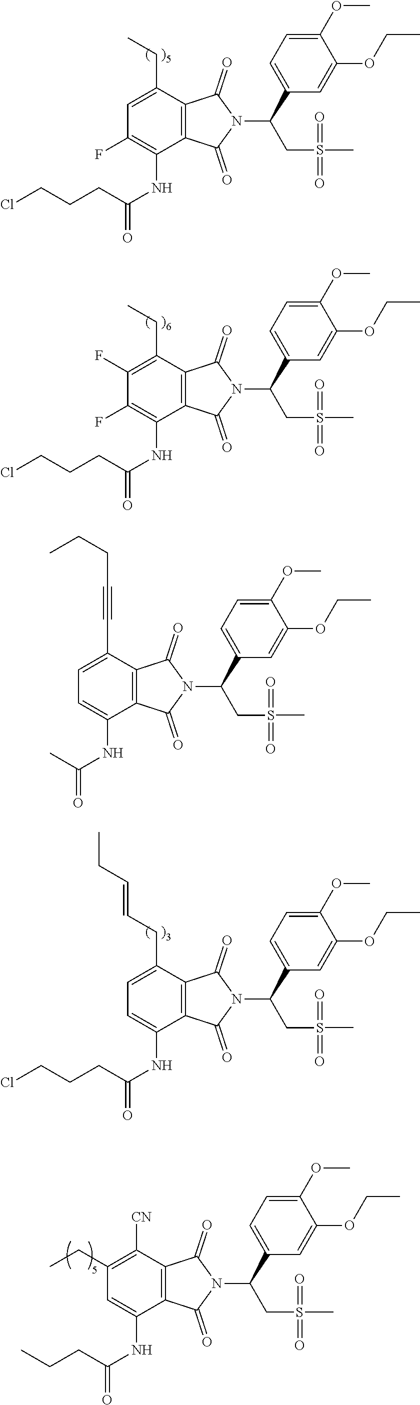

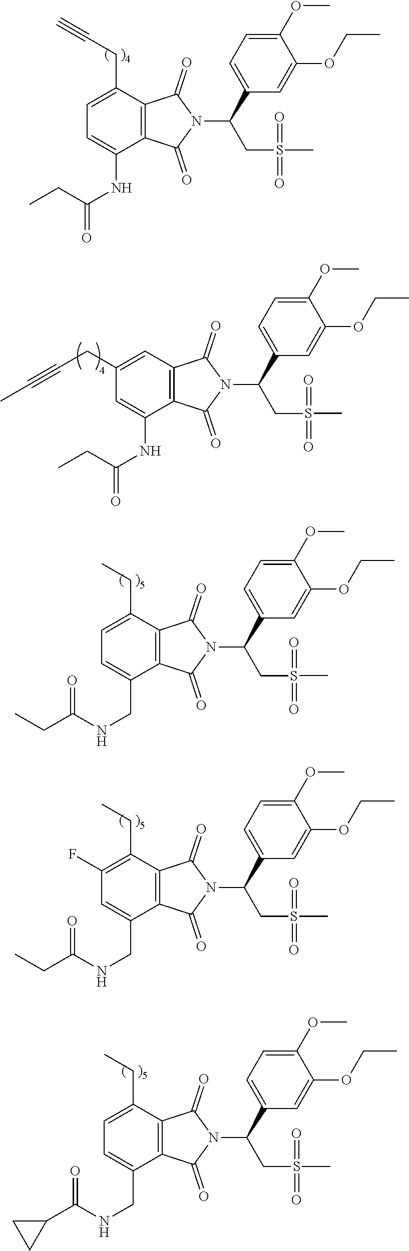

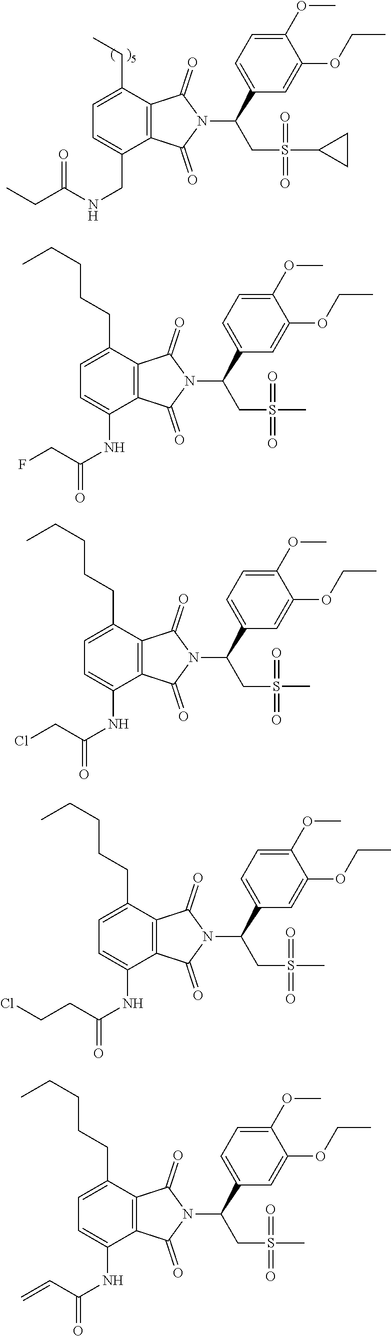

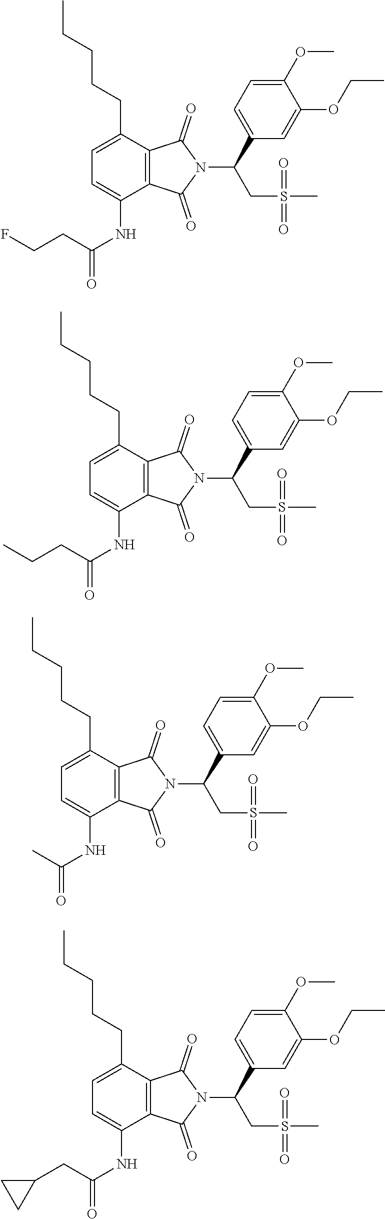

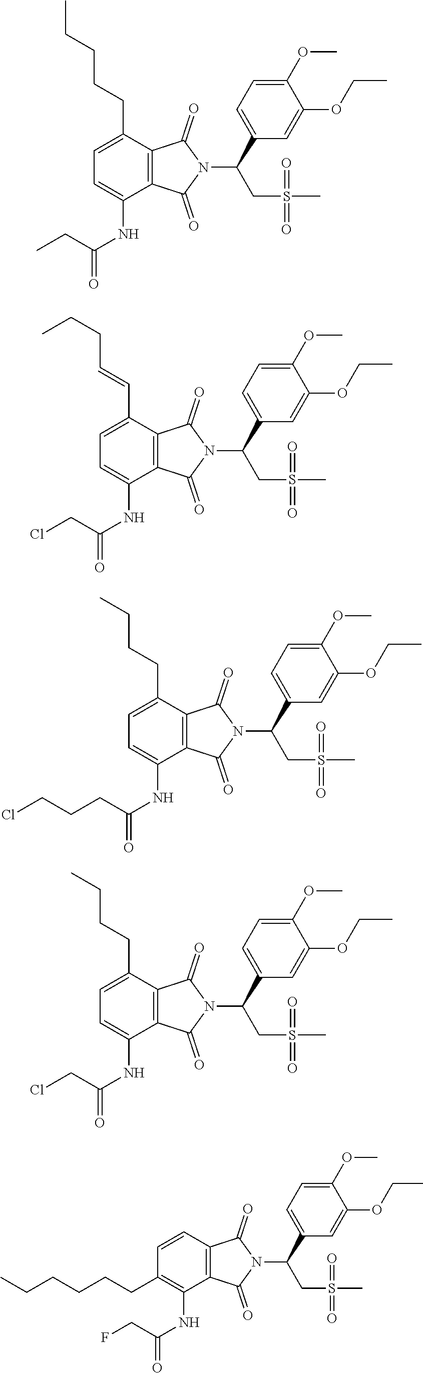

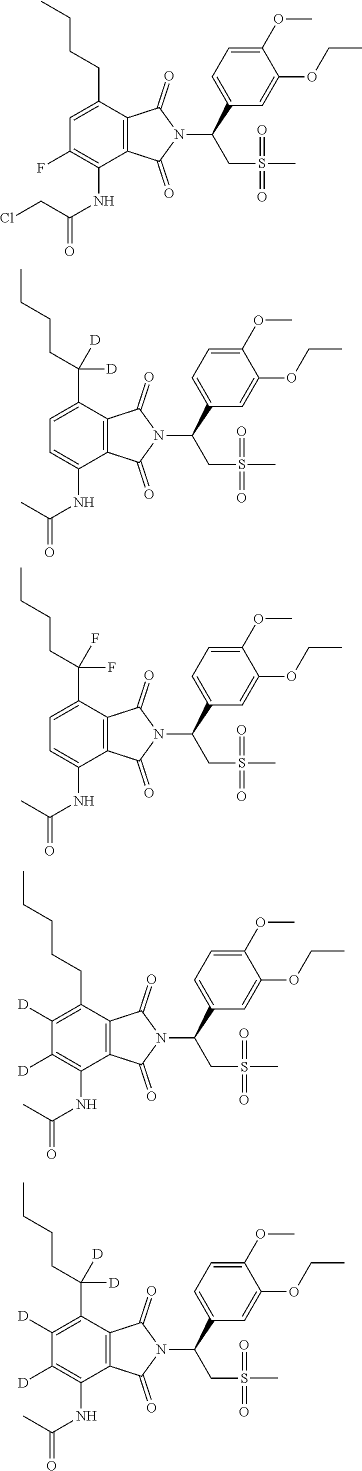

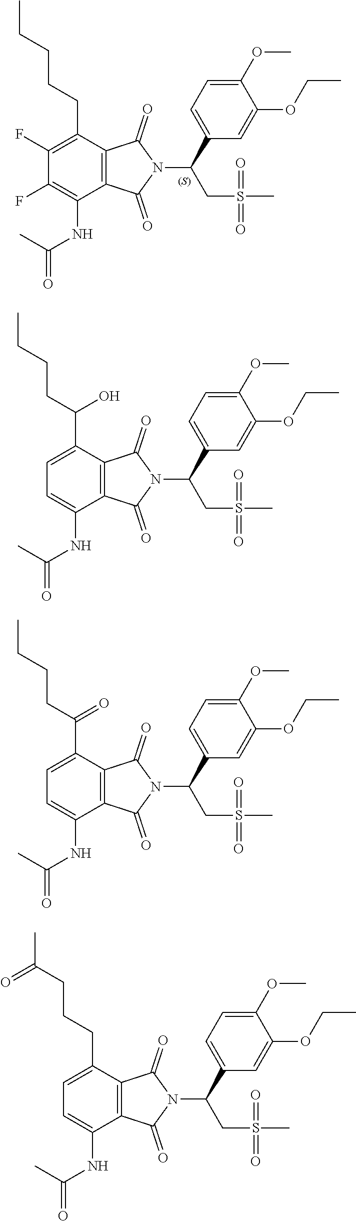

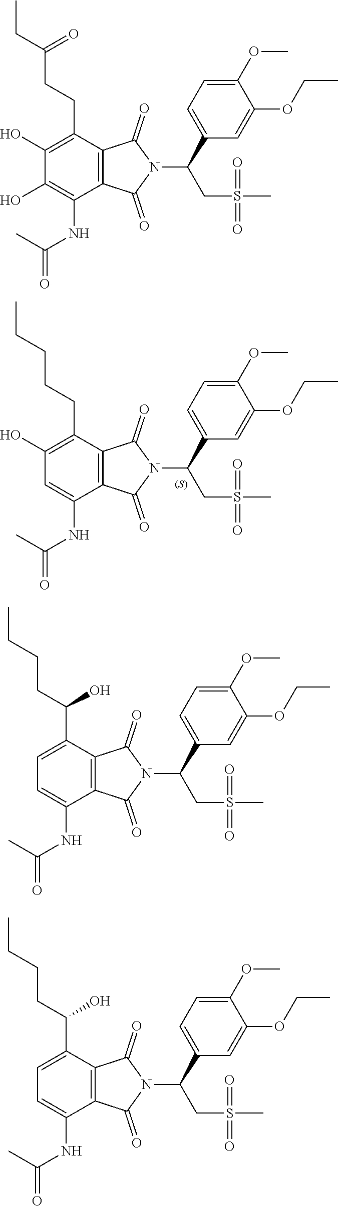

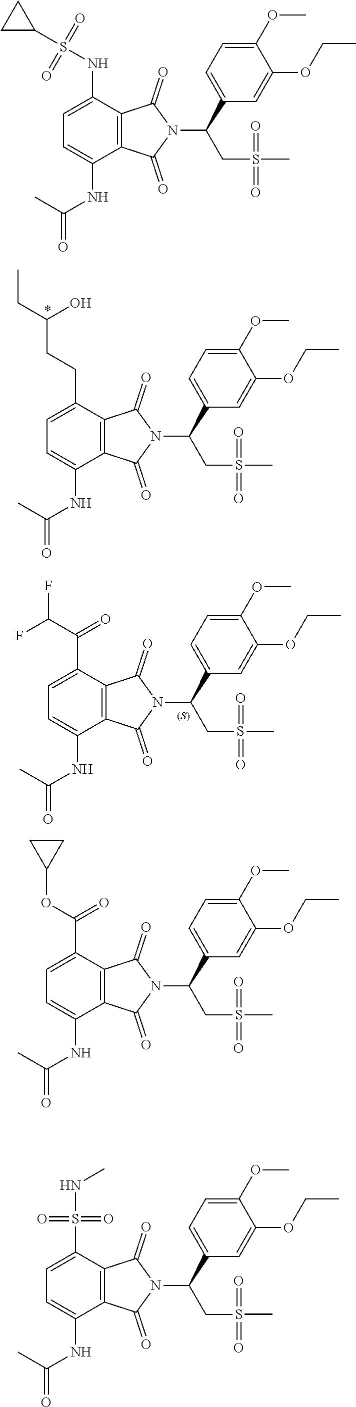

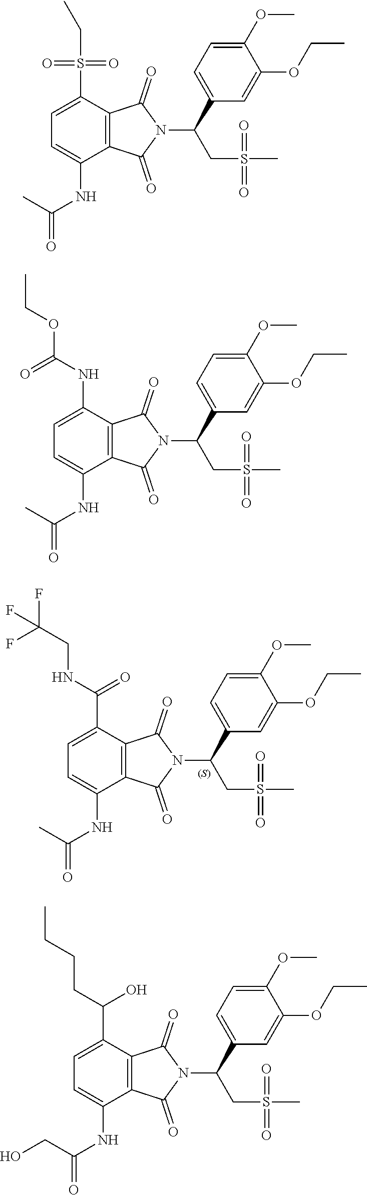

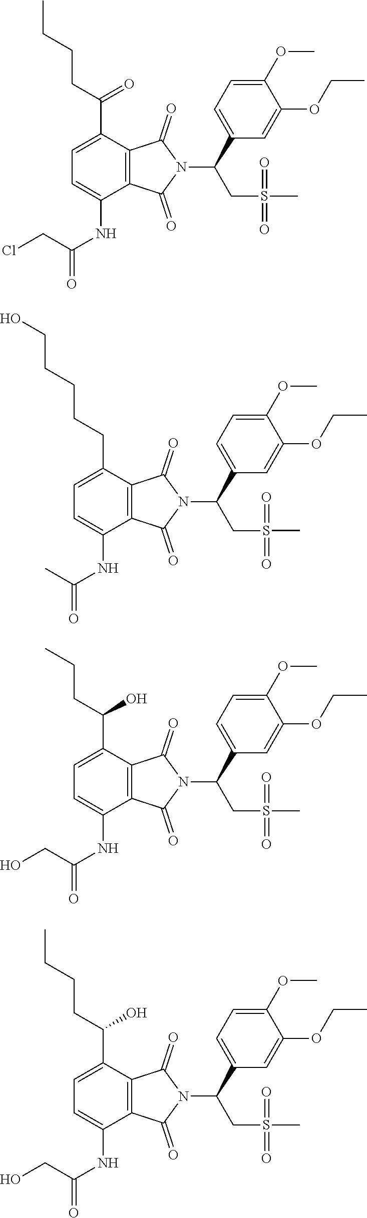

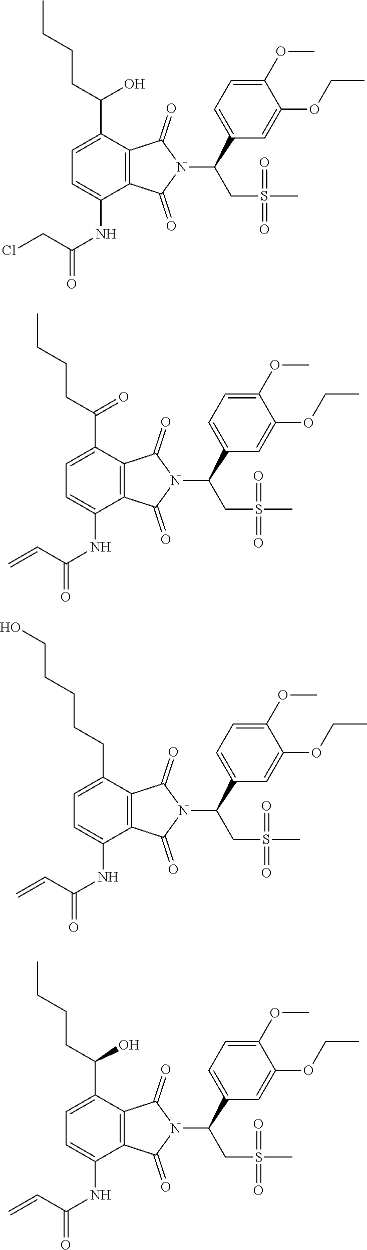

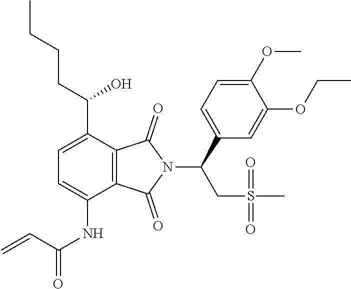 [SMILES: C=CC(=O)Nc1ccc([C@@H](O)CCCC)c2c1C(=O)N([C@H](CS(C)(=O)=O)c1ccc(OC)c(OCC)c1)C2=O]

The present disclosure also provides preparation methods the compound as shown in formula I (including formula II-III) and its racemates, stereoisomers, tautomers, isotopic labels, solvates, polymorphs, esters, prodrugs or pharmaceutically acceptable salts, but not limited to the methods described below. All raw materials are prepared based on the group characteristics of target molecules that conform to the general formula law, and through solutions in these routes and methods well known to the those having ordinary skill in the art of organic chemistry, or are purchased directly. The compound provided by the present disclosure can be synthesized using the methods described below in combination with synthetic methods known in the art of synthetic organic chemistry, or with relevant modifications recognized by those skilled in the art. Those skilled in the art know that, according to a specific target structure, one or more of the following solutions may be optionally combined, or any steps in one or more of the solutions may be combined, so as to obtain a synthetic solution.

The preparation method of the compound as shown in formula I of the present disclosure includes: under suitable conditions, a substituted benzoic acid raw material I-1 (with R′ being halogen, alkane, carboxyl, cyano, amino or nitro, and t being an integer ranging from 1 to 5) is converted into acid anhydride I-2 through synthesis, and then further reacts with an amine intermediate I-3 to generate substituted isoindoline-1, 3-diketone I-4; and under suitable conditions, the compound as shown in formula I is obtained through the steps of adding protecting groups, removing protecting groups, substituting, condensing, reductive amination or hydrolysis. Specifically, the compound can be synthesized with reference to the following further solution.

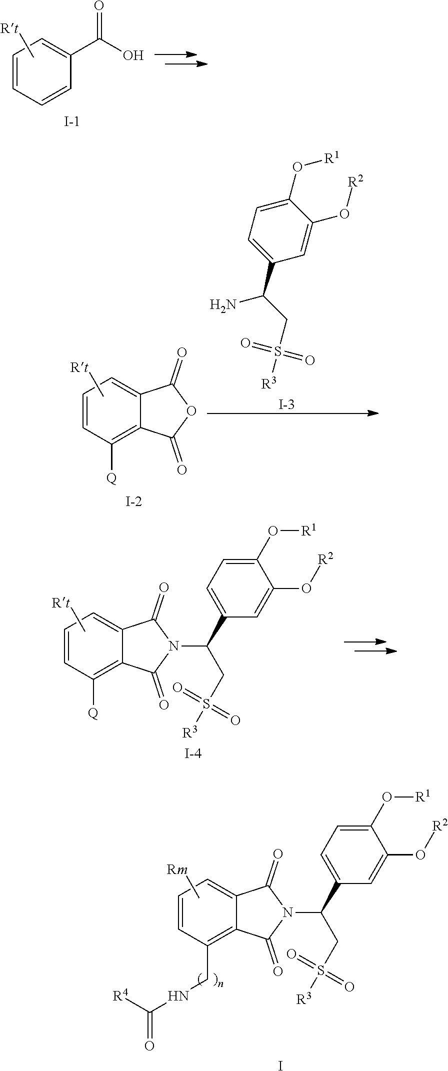

The preparation of the compound provided by the present disclosure may include one or more of the following general steps according to the known synthesis method (such as WO2016169533). Further synthetic route of intermediate sulfonyl ethylamine I-3 (11):

A raw material substituted benzoate 1 is subjected to para-phenolic group protection to obtain compound 2, subjected to meta-etherification to obtain compound 3, and subjected to para-phenolic group deprotection to obtain compound 4. Similar etherification is performed on para-substituted compound 5, ester is reduced to alcohol 6, and oxidation is carried out to obtain intermediate aldehyde 7. Further reaction is carried out to successfully obtain methylsulfonyl styrene 8, which is further alkylated to obtain alkylsulfonyl styrene 9, and subjected to an amination reaction to obtain a product 10. Through chiral resolution or separation of phenyl ketone, (S)-1-(3-alkoxy-4-alkoxyphenyl)-2-alkyl sulfonyl ethylamine 11 can be obtained.

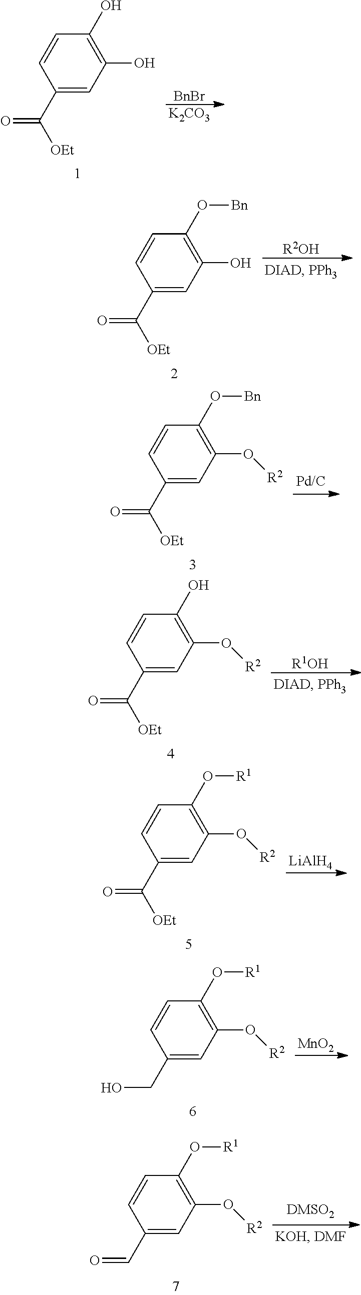

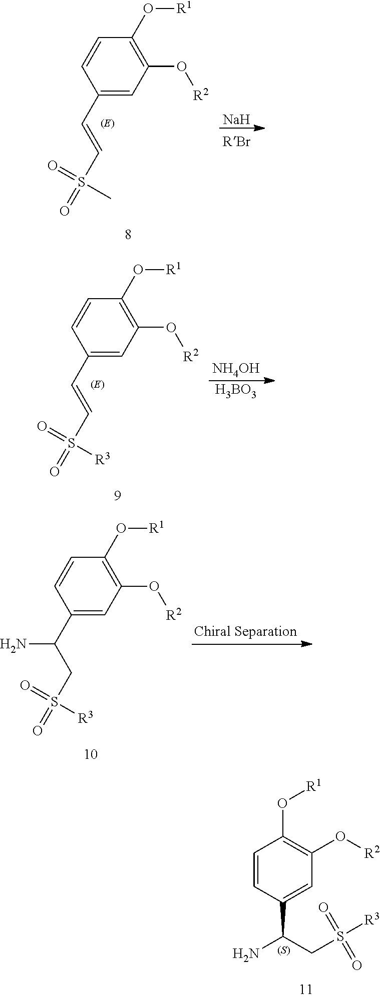

In addition, a raw material phenyl cyanide 12 may also be converted into phenyl ketone 14 intermediate through an intermediate 13, reduced to alcohol 15, and dehydrated to obtain methylsulfonyl styrene 8, so that chiral 1-(3-alkoxy-4-alkoxyphenyl)-2-alkyl sulfonyl ethylamine 11 is obtained by further chemical conversion as above.

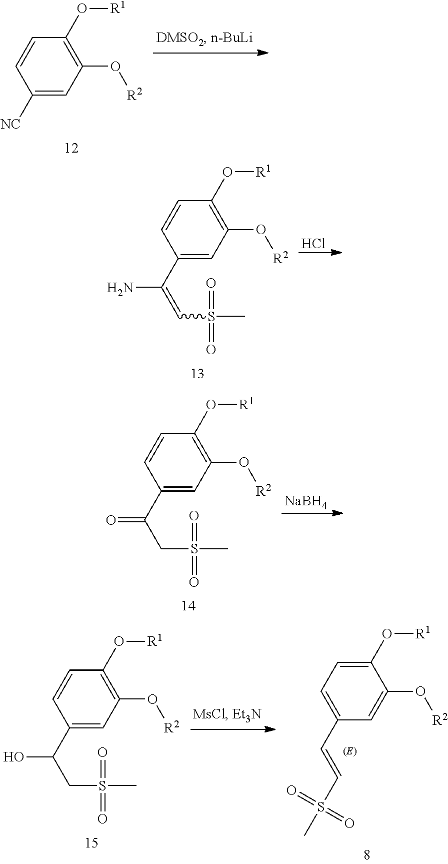

Bihydroxy-substituted benzophenone 24 is para-alkylated to obtain 17, further alkylated to produce bisalkoxy benzophenone 18, subjected to a bromination reaction to obtain bromo-benzophenone 19, and subjected to thioetherification to obtain an intermediate 20. The intermediate 11 is synthesized through two pathways in 21, 22, and 23.

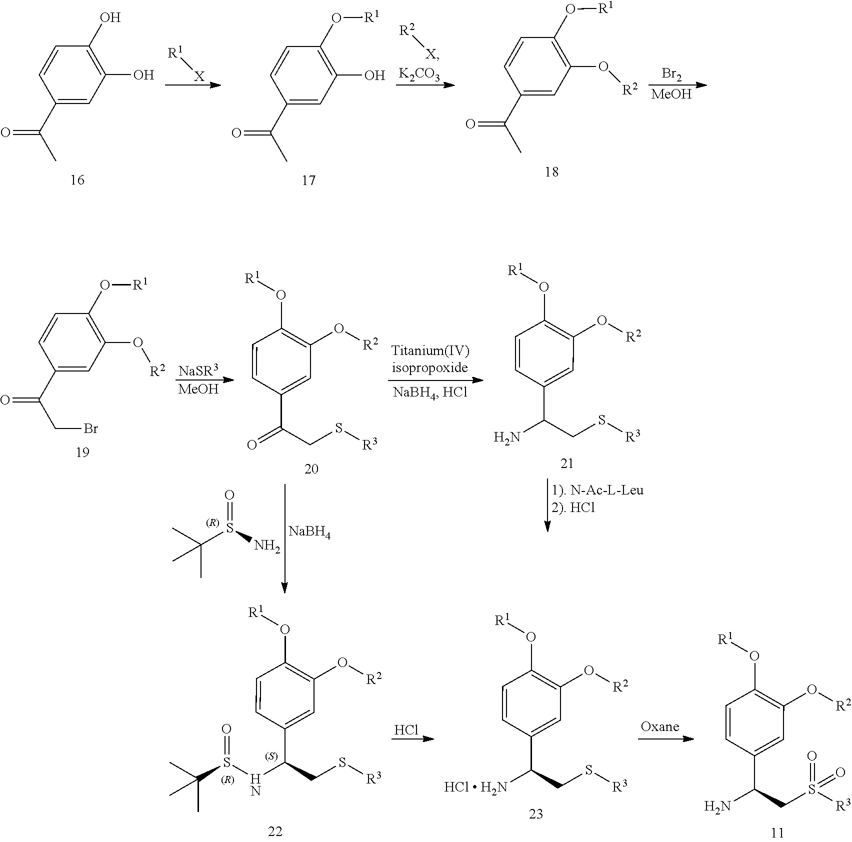

R¹, R² and R³ are as defined in the aforementioned formula I, and X is selected from halogen.

Corresponding chiral compounds can be separated from their racemic compounds by techniques available in the art. Examples include, but are not limited to, chiral salt formation, the use of chirality and high performance liquid chromatography (HPLC), aswell as the formation and crystallization of chiral salts. See, e.g., Jacques, J. et al., Enantiomers, Racemates and Resolutions (Wiley-Interscience, New York, 1981); Wilen, S. H. et al., Tetrahedron, 33:2725 (1977); Eliel, E.L., Stereochemistry of Carbon Compounds (McGraw-Hill, New York, 1962) and Wilen, S. H., Tables of Resolving Agents and Optical Resolutions, 268 (written by Eliel E. L., Notre Dame University Press, Notre Dame, IN, 1972).

In specific examples, chiral amino acid salts of (S)-2-(3-alkoxy-4-alkoxyphenyl)-1-(alkylsulfonyl)-ethylenediamine 11 include, but are not limited to, salts formed with L-isomers of amino acids or L-isomers of acylated amino acids.

According to the embodiments of the present disclosure, the compound provided by the present disclosure can be synthesized according to one selected from the following synthetic routes (referring to WO2018157779A1):

Synthetic route 1:

Halogenated o-methyl benzoic acid 24 undergoes a nitration reaction to generate 25, undergoes an oxidation reaction to obtain substituted phthalic acid 26, then undergoes an anhydride reaction to obtain halogenated 4-nitrobenzoic anhydride 27, and further reacts with 1-(3-alkoxy-4-alkoxyphenyl)-2-alkylsulfonyl ethylamine 11 in acetic acid to obtain halogenated (S)-2-[1-(3-alkoxy-4-alkoxyphenyl)-2-alkylsulfonyl ethyl]-4-nitroisoindoline-1, 3-diketone 28; a nitro group is further reduced to obtain an intermediate 29, which is subjected to acylation to obtain 30; and long-chain hydrocarbon-substituted (S)-2-[1-(3-alkoxy-4-alkoxyphenyl)-2-alkylsulfonyl ethyl]-4-amidoisoindoline-1, 3-diketone II is generated by a Suzuki reaction or Sonogashira reaction.

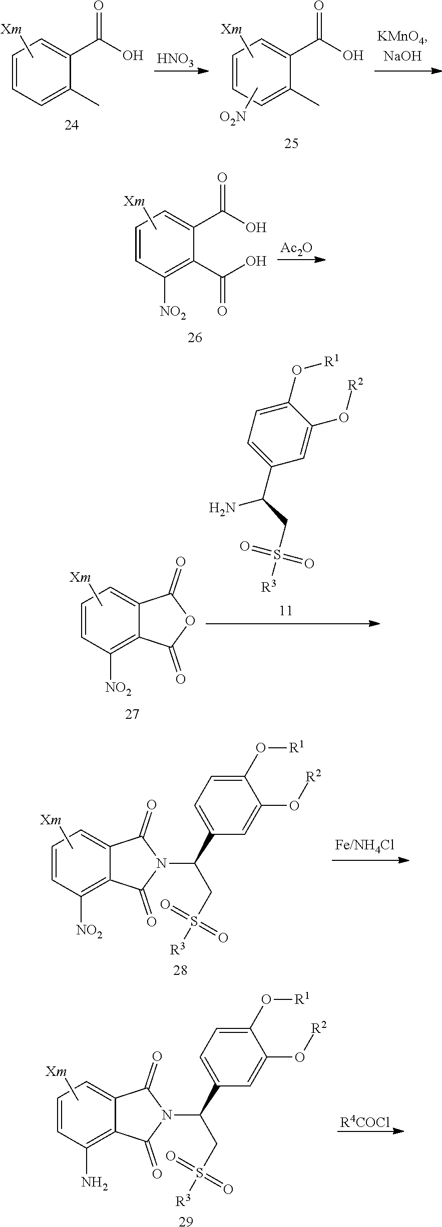

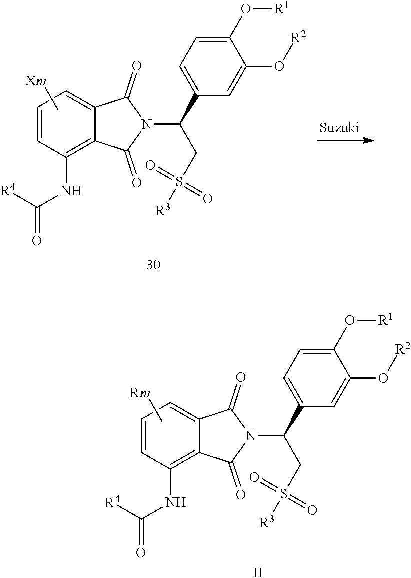

R, R¹, R² R³, R⁴, and m are as defined in the aforementioned formula I, and X is selected from halogen (Cl, Br, I).

Synthetic route 2:

Halogenated 3-nitrobenzoic anhydride 27 and a nitro group are reduced to obtain an intermediate 31 and 4-acylated benzoic anhydride 32, and further reacts with 1-(3-alkoxy-4-alkoxyphenyl)-2-alkylsulfonyl ethylamine 11 in acetic acid to obtain halogenated (S)-2-[1-(3-alkoxy-4-alkoxyphenyl)-2-alkylsulfonyl ethyl]-4-amidoisoindoline-1, 3-diketone 30; and long-chain hydrocarbon-substituted (S)-2-[1-(3-alkoxy-4-alkoxyphenyl)-2-alkylsulfonyl ethyl]-4-amidoisoindoline-1, 3-diketone II is generated by a Suzuki reaction or Sonogashira reaction.

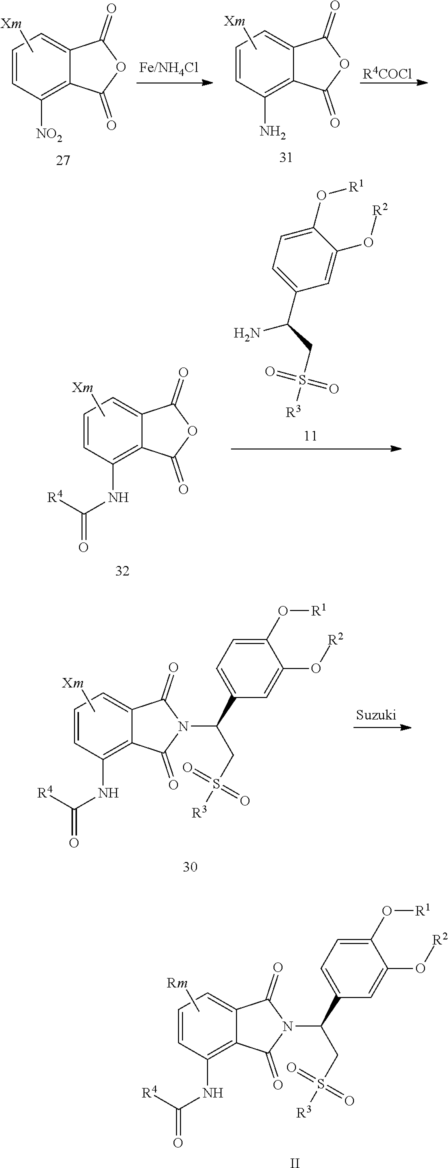

R, R¹, R² R³, R⁴, and m are as defined in the aforementioned formula I, and X is selected from halogen (Cl, Br, I).

Synthetic route 3:

Hydrocarbonated 4-nitrobenzoic anhydride 33 further reacts with 1-(3-alkoxy-4-alkoxyphenyl)-2-alkylsulfonyl ethylamine 11 in acetic acid to obtain hydrocarbonated (S)-2-[1-(3-alkoxy-4-alkoxyphenyl)-2-alkylsulfonyl ethyl]-4-nitroisoindoline-1, 3-diketone 34; and a nitro group is reduced to obtain an intermediate (S)-2-[1-(3-alkoxy-4-alkoxyphenyl)-2-alkylsulfonyl ethyl]-4-amidoisoindoline-1, 3-diketone 35, which is subjected to acylation to obtain (S)-2-[1-(3-alkoxy-4-alkoxyphenyl)-2-alkylsulfonyl ethyl]-4-amidoisoindoline-1, 3-diketone II.

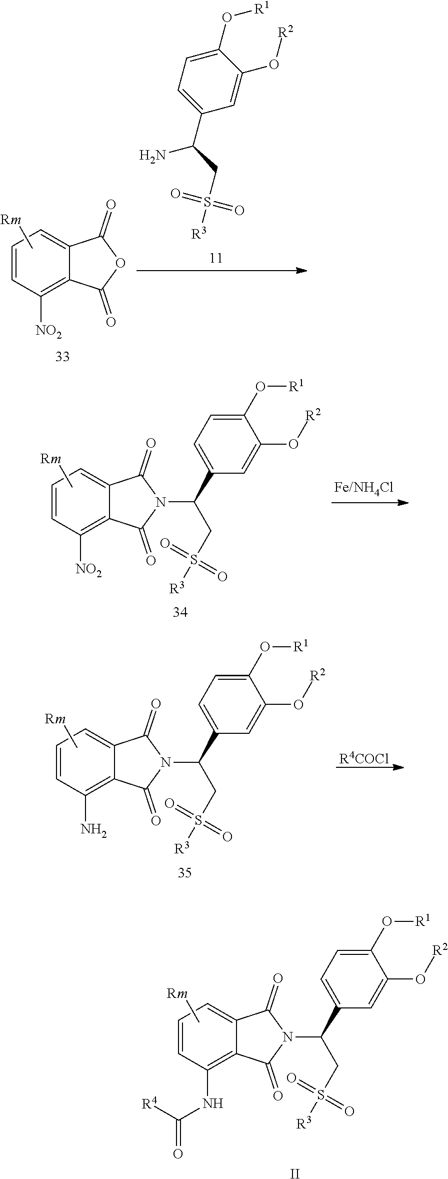

R, R¹, R² R³, R⁴, and m are as defined in the aforementioned formula I.

Synthetic route 4:

Hydrocarbonated 4-aminobenzoic anhydride 36 is acylated to obtain an intermediate 37, and further reacts with 1-(3-alkoxy-4-alkoxyphenyl)-2-alkylsulfonyl ethylamine 11 in acetic acid to obtain chain hydrocarbon-substituted (S)-2-[1-(3-alkoxy-4-alkoxyphenyl)-2-alkylsulfonyl ethyl]-4-amidoisoindoline-1, 3-diketone II.

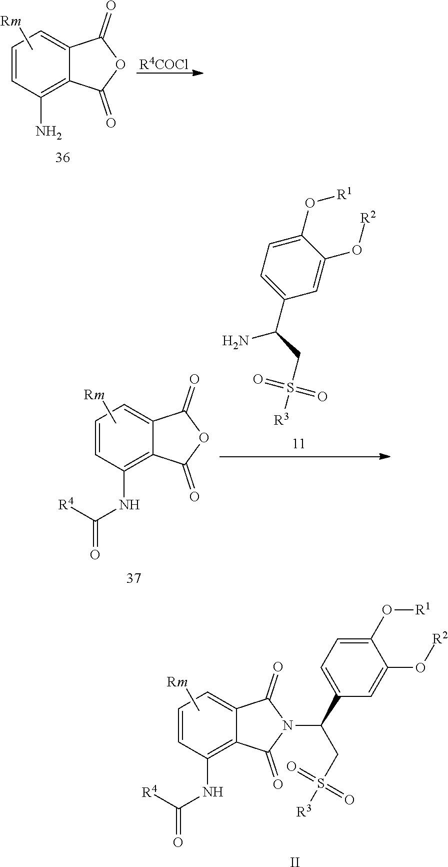

R, R¹, R² R³, R⁴, and m are as defined in the aforementioned formula II.

Synthetic route 5:

4-nitrobenzoic anhydride 27 is aminated to obtain 38, and is subjected to a Mitsunobu reaction with alcohol 39 to obtain an intermediate 28; and after a Suzuki reaction or Sonogashira reaction, an intermediate 34 is generated, which is further reduced to obtain an amino group 35, and then subjected to an acylation reaction to obtain chain hydrocarbon-substituted (S)-2-[1-(3-alkoxy-4-alkoxyphenyl)-2-alkylsulfonyl ethyl]-4-amidoisoindoline-1, 3-diketone II.

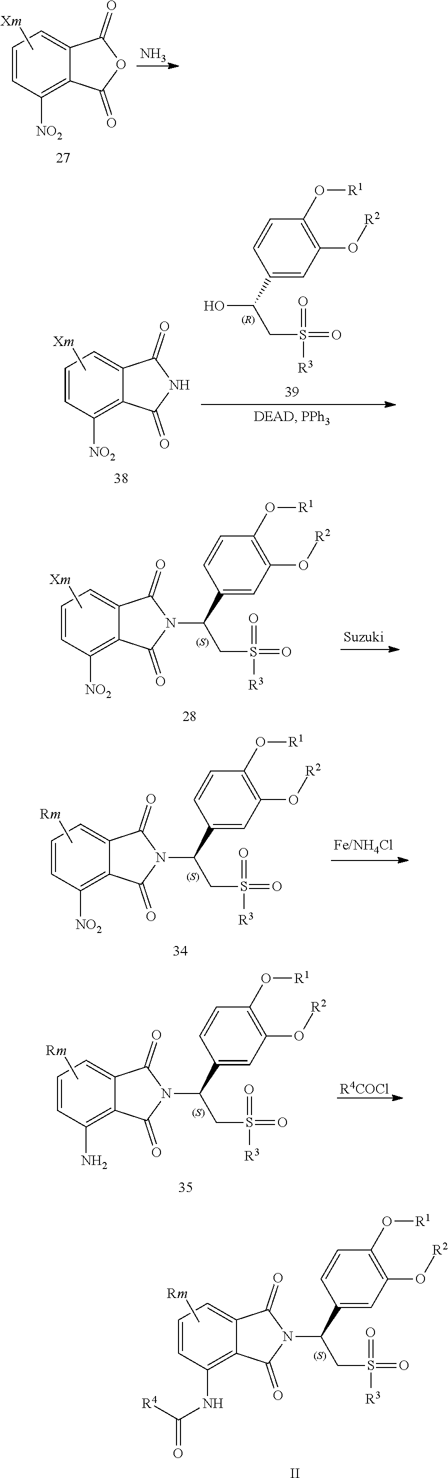

R, R¹, R² R³, R⁴, and m are as defined in the aforementioned formula II.

Synthetic route 6:

Hydrocarbonated (S)-2-[1-(3-alkoxy-4-alkoxyphenyl)-2-alkylsulfonyl ethyl]-4-aminoisoindoline-1, 3-diketone 35 is brominated to obtain 39, cyanated to obtain a cyano compound 40, reduced to obtain a 4-aminomethyl substituted group intermediate 41, and acylated to obtain chain hydrocarbon-substituted (S)-2-[1-(3-alkoxy-4-alkoxyphenyl)-2-alkylsulfonyl ethyl]-4-(amidomethyl) isoindoline-1, 3-diketone.

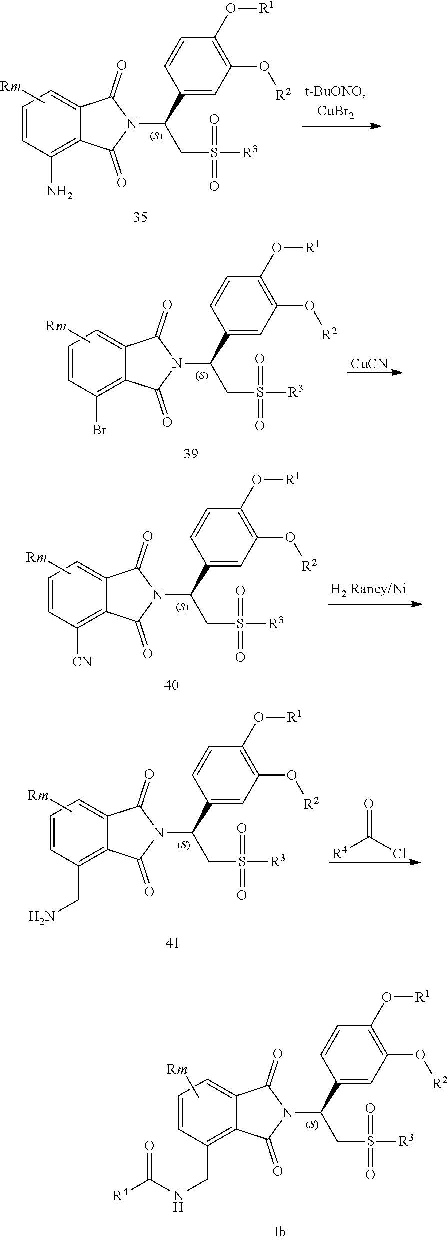

R, R¹, R² R³, R⁴, and m are as defined in the aforementioned formula I.

The present disclosure further provides a pharmaceutical composition, which contains the compound as shown in formula I and its racemates, stereoisomers, tautomers, isotopic labels, solvates, polymorphs, esters, prodrugs or pharmaceutically acceptable salts.

In some embodiments, the pharmaceutical composition according to the present disclosure contains a therapeutically effective amount of the compound as shown in formula I and its racemates, stereoisomers, tautomers, isotopic labels, solvates, polymorphs, metabolites, esters, prodrugs, or pharmaceutically acceptable salts and pharmaceutically acceptable carriers.

The carrier in the pharmaceutical composition is “acceptable”, which is compatible with (and preferably, is capable of stabilizing) the active components of the composition and is not deleterious to the subject being treated. One or more solubilizers may be used as pharmaceutical excipients for delivery of active compounds.

The present disclosure further provides the use of the compound as shown in formula I and its racemates, stereoisomers, tautomers, isotopic labels, solvates, polymorphs, esters, prodrugs or pharmaceutically acceptable salts, or the pharmaceutical composition in the preparation of medicines for inhibiting PDE4 enzyme.

The present disclosure further provides the use of the compound as shown in formula I and its racemates, stereoisomers, tautomers, isotopic labels, solvates, polymorphs, esters, prodrugs or pharmaceutically acceptable salts, or the pharmaceutical composition in the preparation of medicines for treating diseases related to the regulation of intracellular cAMP level.

The present disclosure further provides the use of the compound as shown in formula I and its racemates, stereoisomers, tautomers, isotopic labels, solvates, polymorphs, esters, prodrugs or pharmaceutically acceptable salts, or the pharmaceutical composition in the preparation of medicines for inhibiting the production of TNF-α or NF-κB.

According to the embodiments of the present disclosure, the conditions ameliorated by by inhibiting the PDE4 with the medicines for inhibiting the PDE4 include, but are not limited to, dermatitis, psoriasis, atopic dermatitis, seborrheic dermatitis, stasis dermatitis, palmoplantar abscess, asthma, inflammation (e.g., inflammation caused by reperfusion), chronic or acute obstructive pulmonary disease, chronic or acute pneumonia, pulmonary diseases caused by viruses such as Covid-19, enteritis, segmental ileitis, psoriasis, psoriatic arthritis, Behcet's disease or colitis.

In particular method of the present disclosure, the compound provided by the present disclosure, or the pharmaceutically acceptable polymorph, prodrug, salt, solvate, hydrate or clathrate thereof, is administered in combination with at least one another therapeutic agent.

The unit dosage forms of the present disclosure are suitable for oral, mucosal (e.g., nasal, sublingual, vaginal, buccal or rectal), parenteral (e.g., subcutaneous, intravenous, single bolus injection, intramuscular or arterial) or transdermal administration to patients, as well as topical dosage forms in the form of topical or inhalants. The dosage forms include, but are not limited to: tablets, pills, caplets, sustained release dosage forms, capsules such as soft elastic gelatin capsules, cachets, troches, dispersants, suppositories, ointments, cataplasm (poultices), plasters, powders, dressings, creams, medicinal paste, solutions, patches, aerosols (such as nasal sprays or inhalants), gels, dry powder inhalants, liquid dosage forms suitable for oral or transmucosal administration to patients, including suspensions (such as aqueous or non-aqueous suspensions, oil-in-water emulsions, or water-in-oil liquid emulsions), solutions, and elixirs, liquid dosage forms suitable for parenteral administration to patients, and sterile solid dosage forms (e.g., crystalline or amorphous solids) that can be reconstituted to provide liquid dosage forms suitable for parenteral administration to patients. The specific dosage forms encompassed by the present disclosure vary in various aspects, which will be apparent to those skilled in the art. See, for example, Remington's Pharmaceutical Sciences, 18th edition, published by Mack, Easton PA (1990).

Terminology:

Unless otherwise stated, definitions of groups and terms set forth in the Description and Claims of the present application, including their definitions as examples, exemplary definitions, preferred definitions, definitions set forth in tables, definitions of specific compounds described in the examples etc., may be arbitrarily combined and bonded with each other. The group definitions and compound structures after such combinations and bindings shall fall within the scope contained in the Description of the present application.

The term “halogen” refers to F, Cl, Br and I. In other words, F, Cl, Br, and I may be described as “halogen” in this Description.

Optionally substituted with substituents described herein encompasses both unsubstituted as well as substituted with one or more substituents, e.g., “optionally substituted with one, two or more R” means that it may be not substituted with R (unsubstituted) or substituted with one, two or more R.

The term “hydrocarbyl groups” include saturated or unsaturated chain or cyclic hydrocarbyl groups with straight or branched chains, the types of which may be selected from alkyl groups, alkenyl groups, alkynyl groups, etc., and the number of carbon atoms of each of the hydrocarbyl groups (alkyl groups, alkenyl groups, alkynyl groups) is preferably 1-16, more preferably 1-12, 1-8, 5-8, 1-5, 1-3, etc. Specific groups may be included, but not limited to the followings: methyl, cthyl, n-propyl, isopropyl, n-butyl, isobutyl, tert-butyl, n-pentyl, isopentyl, neopentyl, n-hexyl, vinyl, 1-propenyl, 2-propenyl, 1-methylvinyl, 1-butenyl, 1-ethylvinyl, 1-methyl-2-propenyl, 2-butenyl, 3-butenyl, 2-methyl-1-propenyl, 2-methyl-2-propenyl, 1-pentenyl, 1-hexcnyl, cthynyl, 1-propynyl, 2-propynyl, 1-butynyl, 1-methyl-2-propynyl, 3-butynyl, 1-pentynyl, 1-hexynyl, cyclopropyl, cyclobutyl, cyclopentyl, and cyclohexyl; and the hydrocarbyl groups in other terms (including alkyl groups, alkenyl groups, and alkynyl groups) also conforms to this definition.

The term “heteroalkyl group” by itself or in combination with another term represents a stable straight-chain or branched-chain alkyl atom group or its combination, composed of a certain number of carbon atoms and at least one heteroatom. The number of the carbon atoms may be 1-16, for example, 1, 2, 3, 4, 5, 6, 7, 8, 9, 10, 11, 12, 13, 14, 15, 16. The heteroalkyl group may optionally contain one, two, or more heteroatoms selected from N, O, S (or be interpreted as optional heteroatoms inserted into either a C—C bond or a C—H bond in an alkyl group). The heteroatoms O, N, and S may be located at any internal positions of the heteroalkyl group or at the positions where alkyl groups attach to the rest of a molecule. Examples include but are not limited to —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH—CH—O—CH₃, —CH₂—CH=N—OCH₃, and —CH=CH—N(CH₃)—CH₃. At most two heteroatoms may be consecutive, e.g., —CH₂—NH—OCH_{3.}

The term “cycloalkyl group”, including “cycloalkyl groups of C₃₋₁₂”, should be understood to refer to a saturated or unsaturated monovalent mono- or bicyclic ring having 3-12 carbon atoms, preferably cycloalkyl groups of C₃₋₈, and more preferably cycloalkyl groups of C₃₋₆. For example, the cycloalkyl groups of C₃₋₈ should be understood to refer to saturated or unsaturated monovalent monocyclic or bicyclic rings having 3, 4, 5, 6, 7 or 8 carbon atoms. The cycloalkyl groups of C₃₋₁₂ may be monocyclic hydrocarbyl groups, such as cyclopropyl, cyclobutyl, cyclopentyl, cyclohexyl, cycloheptyl, cyclooctyl, cyclononyl or cyclodecyl, or may be bicyclic hydrocarbyl groups such as tetrahydronaphthalene or decahydronaphthalenc.

The term “3-12 membered heterocyclic groups” should be understood to refer to saturated monovalent monocyclic, bicyclic hydrocarbon-ring or bridged alkane, including 1-5 non-aromatic cyclic groups having heteroatoms independently selected from N, O, and S, with a total number of ring formation atoms being 3-12 (e.g., 3, 4, 5, 6, 7, 8, 9, 10, 11, 12), preferably “3-10 membered heterocyclic groups”. The term “3-10 membered heterocyclic groups” refers to saturated monovalent monocyclic, bicyclic hydrocarbon-ring or bridged alkane, which includes 1-5, preferably 1-3 heteroatoms independently selected from N, O, and S, for example, 1, 2, and 3 heteroatoms independently selected from N, O, and S. The heterocyclic groups may be attached to the rest of a molecule through any of the carbon atoms or a nitrogen atom (if present). In particular, the heterocyclic groups may include but are not limited to: 4-membered rings, such as azetidinyl, and oxetanyl; 5-membered rings, such as tetrahydrofuranyl, dioxolenyl, pyrrolidinyl, imidazolidinyl, pyrazolidyl, and pyrrolinyl; 6-membered rings, such as tetrahydropyranyl, piperidinyl, morpholinyl, dithianyl, thiomorpholinyl, piperazinyl or trithianyl; or 7-membered rings, such as diazacycloheptyl. Optionally, the heterocyclyl groups may be benzo-fused. The heterocyclyl groups may be bicyclic, including, but not limited to, 5, 5-membered rings, such as a hexahydrocyclopento [c] pyrrol-2 (1H)-yl ring, or a 5,6-membered bicyclic ring, such as a hexahydropyrrolo [1, 2-a] pyrazin-2 (1H)-yl ring. The nitrogen atom-containing ring may be partially unsaturated, i.e., it may contain one or more double bonds, including, but not limited to, 2, 5-dihydro-1H-pyrrolyl, 4H-[1, 3, 4] thiadiazinyl, 4, 5-dihydrooxazolyl or 4H-[1, 4] thiazinyl, or it may be benzo-fused, including, but not limited to, dihydroisoquinolinyl. According to the present disclosure, the heterocyclic groups are non-aromatic. When the 3-12 membered heterocyclic groups are attached to other groups to form the compound provided by the present disclosure, the carbon atoms on the 3-12 membered heterocyclic groups may be attached to other groups, or the heterocyclic atoms on the 3-12 membered heterocyclic groups may be attached to other groups. For example, when the 3-12 membered heterocyclic group is selected from a piperazinyl group, the nitrogen atom of the piperazinyl group may be attached to other groups. Alternatively, when the 3-12 membered heterocyclic group is selected from a piperidinyl group, the nitrogen atom on the piperidinyl ring and the carbon atom in its para position may be attached to other groups.

The term “aryl groups of C₆₋₁₄” should be understood to refer to aromatic or partially aromatic monovalent monocyclic, bicyclic or tricyclic hydrocarbon rings having 6 to 14 carbon atoms, such as an aromatic or partially aromatic monovalent monocyclic, bicyclic or tricyclic hydrocarbon ring having 6, 7, 8, 9, 10, 11, 12, 13 or 14 carbon atoms, in particular a ring (“aryl group of C₆”) having 6 carbon atoms, i.e., phenyl; or biphenyl, or a ring (“aryl group of C₉”) having 9 carbon atoms, such as indanyl or indenyl, or a ring (“aryl group of C₁₀”) having 10 carbon atoms, such as tetrahydronaphthyl, dihydronaphthyl or naphthyl, or a ring (“aryl group of C₁₃”) having 13 carbon atoms, such as fluorenyl, or a ring (“aryl group of C₁₄”) having 14 carbon atoms, such as anthryl. When the aryl groups of C₆₋₂₀ are substituted, it may be mono-or poly-substituted. Also, the substitution site thereof is not limited, and may be, for example, ortho-, para-, or meta-substitution.

The term “5-14 membered heteroaryl groups” or “5-14 membered heteroaryl groups” should be understood to include such monovalent monocyclic, bicyclic or tricyclic aromatic ring systems, which are aromatic or partially aromatic, have 5 to 14 ring atoms and contain 1 to 5 heteroatoms independently selected from N, O and S. For example, it may have 5, 6, 7, 8, 9, 10, 11, 12, 13 or 14 ring atoms, in particular 5, 6, 9 or 10 carbon atoms, and contain 1 to 5, preferably, 1 to 3 heteroatoms independently selected from N, O and S; and in addition, in each case, it may be benzo-fused. In particular, the heteroaryl groups are selected from the group consisting of thienyl, furyl, pyrrolyl, oxazolyl, thiazolyl, imidazolyl, pyrazolyl, isoxazolyl, isothiazolyl, oxadiazolyl, triazolyl, thiadiazolyl, and thi-4H-pyrazolyl, and benzo derivatives thereof, for example, benzofuryl, benzothienyl, benzoxazolyl, benzisoxazolyl, benzimidazolyl, benzotriazolyl, indazolyl, indolyl, isoindolyl, and the like; or pyridyl, pyridazinyl, pyrimidinyl, pyrazinyl, and triazinyl, and benzo derivatives thereof, such as quinolyl, quinazolinyl, and isoquinolyl; or acridyl, indolazinyl, purinyl, and benzo derivatives thereof; or cinolinyl, phthalazinyl, quinazolinyl, quinoxalinyl, naphthyridinyl, pteridyl, carbazolyl, acridinyl, phenazinyl, phenothiazinyl, phenoxazinyl and the like. When the 5-14 membered heteroaryl groups are attached to other groups to form the compound provided by the present disclosure, the carbon atoms on the 5-14 membered heteroaryl group rings may be attached to other groups, or the heteroatoms on the 5-14 membered heteroaryl group rings may be attached to other groups. When the 5-14 membered heteroaryl groups are substituted, it may be mono-or poly-substituted. Also, the substitution site thereof is not limited, for example, the hydrogen attached to the carbon atoms on the heteroaryl group rings may be substituted, or the hydrogen attached to the heteroatoms on the heteroaryl group rings may be substituted.

Unless otherwise specified, a heterocyclyl, heteroaryl or sub-heteroaryl group includes all possible isomeric forms thereof, such as positional isomers thereof. Thus, for some illustrative non-limiting examples, they may include forms of substitution or bonding to other groups at one, two, or more sites such as 1-, 2-, 3-, 4-, 5-, 6-, 7-, 8-, 9-, 10-, 11-, and 12- (if present), including pyridine-2-yl, pyridylidene-2-yl, pyridine-3-yl, pyridylidene-3-yl, pyridine-4-yl, and pyridylidene-4-yl; thiophene or thenylidene includes thiophene-2-yl, thenylidene-2-yl, thiophene-3-yl, and thenylidene-3-yl; and pyrazole-1-yl, pyrazole-3-yl, pyrazole-4-yl, pyrazole-5-yl.

The term “effective amount” or “therapeutically effective amount” refers to an amount of a compound described herein that is sufficient to effect the intended use, including but not limited to the disease treatment as defined below. The therapeutically effective amount may vary depending on the intended use (in vitro or in vivo), or the subject and disease being treated, such as the weight and age of the subject, the severity of the disease, and the mode of administration, which can be easily determined by a person generally skilled in the art. The specific dosage will vary depending upon the particular compound selected, the dosing regimen pursuant to which it is administered, whether it is administered in combination with other compounds, the timing of administration, the tissue to which it is administered, and the physical delivery system being carried.

The term “solvates” are those forms of the compounds provided by the present disclosure which, in solid or liquid states, form complexes by coordination with solvent molecules. Hydrates are specific forms of the solvates, in which the coordination is achieved in water. In the present disclosure, the preferred solvates are hydrates. Further, the pharmaceutically acceptable solvates (hydrates) of the compound as shown in formula I of the present disclosure refers to co-crystals and clathrates formed by the compound I with one or more molecules of water or other solvents based on stoichiometry. The solvents that can be used for the solvates include, but are not limited to, water, methanol, ethanol, ethylene glycol, and acetic acid.

The term “prodrugs” or “drug precursors” refer to compounds converted in vivo to the compounds as shown in the foregoing general formula or specific compound. Such conversion is affected by hydrolysis of the prodrugs in blood or enzymatic conversion to a parent structure in blood or tissue. The prodrugs described in the present disclosure may be esters. In the present disclosure, the esters that may be used as the prodrugs include phenyl esters, aliphatic esters, acyloxymethyl esters, carbonates, carbamates and amino-acid esters. For example, one of the compounds provided by the present disclosure contains a hydroxy/carboxy group, i.e., it can be acylated to obtain the compound in a prodrug form. Other prodrug forms include phosphate esters, such as those obtained by phosphorylation of the hydroxyl group on the parent.

### Beneficial Effects

The isoindoline-1, 3-diketone compound provided by the present disclosure has a prominent and highly inhibited biological response to PDE4 enzyme, which further increases CAMP level or inhibits factors such as TNF-α, thereby effectively treating psoriasis, psoriatic arthritis, scalp psoriasis, Behcet's disease, atopic dermatitis (AD), vitiligo, seborrheic dermatitis, stasis dermatitis, palmoplantar abscess, chronic obstructive pulmonary disease (COPD), acute pneumonia (ARDS), virus-induced lung diseases, and respiratory inflammatory diseases. The compound provided by the present disclosure has an outstanding inhibitory activity of the isoindoline-1, 3-diketone series compounds on the biological enzyme PDE4, and has the beneficial effect of higher efficacy.

### DETAILED DESCRIPTION

The technical solution of the present disclosure will be described in further detail below with reference to specific examples. It should be understood that the following examples are only exemplary to illustrate and explain the present disclosure, and should not be construed as limiting the scope of the present disclosure. All technologies implemented based on the above content of the present disclosure are covered within the intended protection scope of the present disclosure.

Unless otherwise specified, the raw materials and reagents used in the following examples are commercially available or can be prepared by known methods.

### EXAMPLE 1

### (S)-2-[1-(3-ethoxy-4-methoxyphenyl)-2-alkylsulfonyl ethyl]-4-(γ-chlorobutyramido)-5-hexyl isoindoline-1, 3-diketone

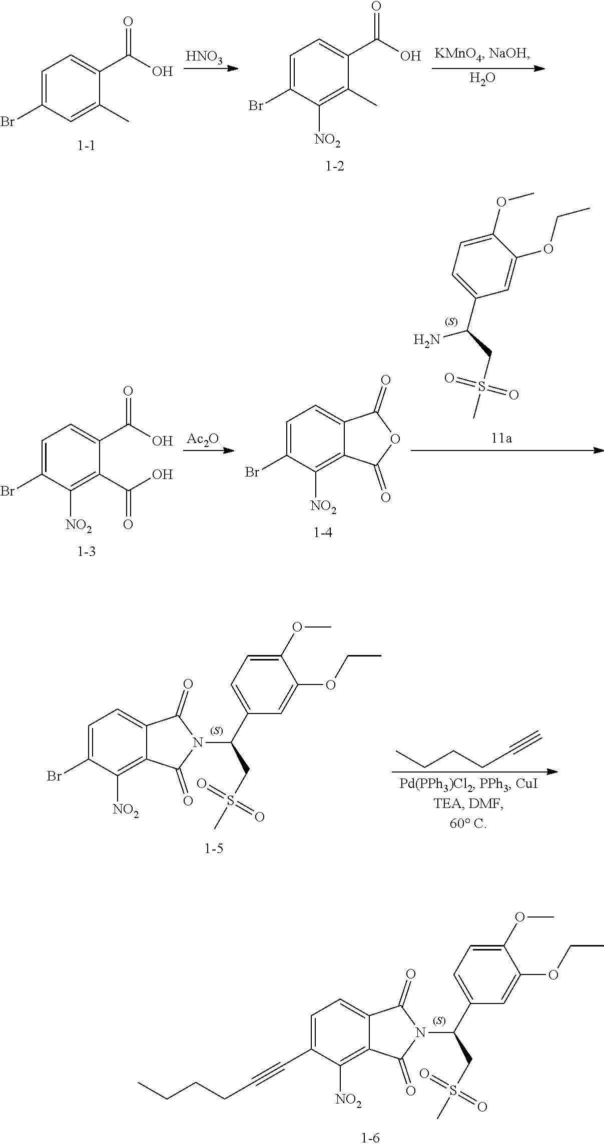

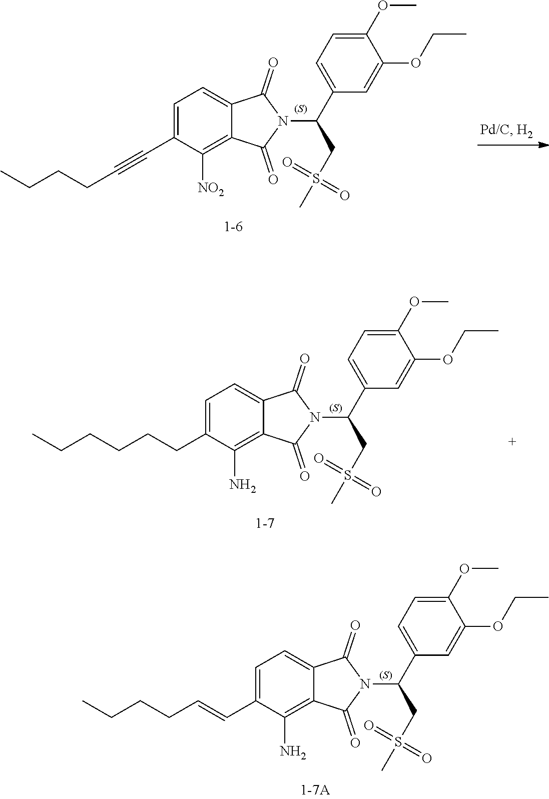

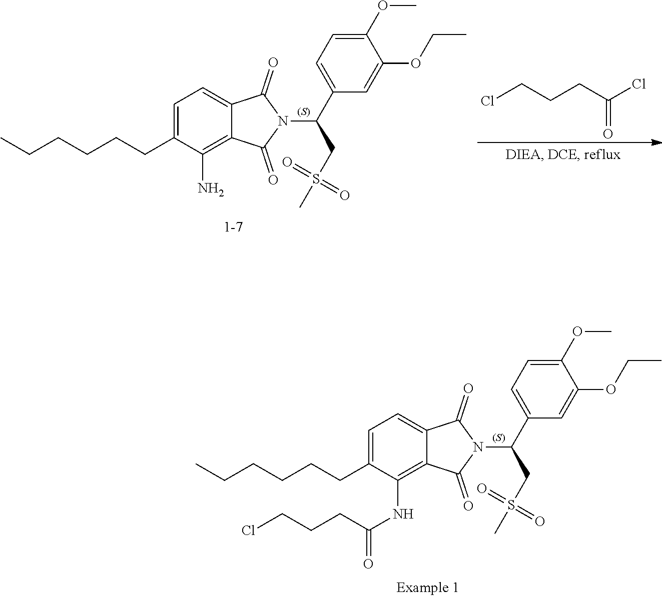

Synthesis of compound 1-2: A compound 1-1 (5 g, 23.25 mmol, 1 eq) was added in batches to fuming nitric acid (1.47 g, 23.25 mmol, 16 mL, 1 eq) at 0° C., after addition, the resulting mixture was further stirred at 0° C. for 1 h, the yellow suspension was formed. The mixture was stirred and poured into ice water (100 mL), the suspension was filtered, the filter cake was washed with water (30 mL), the washed filter cake was dissolved in ethyl acetate (100 mL), and the product was dried with Na₂SO₄ and concentrated in vacuo. A compound 1-2 (5.3 g, crude) was obtained as a yellow solid.

Synthesis of compound 1-3: The compound 1-2 (5.3 g, 6.73 mmol, 1 eq) was dissolved in H₂O (60 mL), NaOH (2.42 g, 60.53 mmol, 9 eq) was added into the solution, and the obtained mixture was heated to 80° C. and added with KMnO₄ (25.51 g, 161.42 mmol, 24 eq) in batches within 3 h. After the addition was completed, stirring was continued for 30 min, suction filtration was carried out, the solid was washed with hot water (30 mL*3), the aqueous phase was cooled with ice water, and the pH was adjusted to 2 with 2M HCl. The product was extracted with ethyl acetate (100 mL*2), washed with a saturated saline solution after combination of organic phases, dried with Na₂SO₄, filtered, and concentrated to obtain a compound 1-3 (1.9 g, crude) as a yellow solid.

Synthesis of compound 1-4: The compound 1-3 (1.9 g, 3.93 mmol, 1 eq) was dissolved in Ac2O (21.80 g, 213.54 mmol, 20 mL, 54.33 eq), stirred at 140° C. for 16 h, and concentrated to obtain a compound 1-4 (1.6 g, crude) as a light brown solid.

Synthesis of compound 1-5: The compound 1-4 (1.6 g, 3.53 mmol, 1 eq) and a compound 11a (1.54 g, 5.65 mmol, 1.6 eq) were dissolved in AcOH (20 mL) and stirred at 120° C. for 18 h. The reaction solution was concentrated to obtain a crude product, and the crude product was purified by silica gel chromatography (petroleum ether:ethyl acetate=10:1 to 1:1) to obtain a compound 1-5 (1.2 g, crude) as a yellow solid.

Synthesis of compound 1-6: The compound 1-5 (1.2 g, 1.50 mmol, 1 eq), PdCl₂(PPh3)2 (210.83 mg, 300.37 μmol, 0.2 eq), CuI (57.21 mg, 300.37 μmol, 0.2 eq), DIEA (582.31 mg, 4.51 mmol, 784.78 μL, 3 eq) and 1-hexyne (370.11 mg, 4.51 mmol, 506.99 μL, 3 eq) were dissolved in DMF (12 mL), the reaction solution was stirred at 60° C. for 18 h under N₂ atmosphere. The reaction mixture was added with water (15 mL) and ethyl acetate (20 mL), the aqueous phase was extracted with ethyl acetate (30 mL*3), the combined organic layers were washed with a saturated saline solution, dried with Na₂SO₄, filtered, and concentrated to obtain a crude product, and the residue was purified by silica gel chromatography (SiO₂, PE:EtOAc=10:1 to 1:1) to obtain a compound 1-6 (376 mg, yield: 47.36%) as a yellow solid.

1H-NMR(400 MHz, CDCl₃) of the compound 1-6: δ=7.87 (d, J=8.0 Hz, 1H), 7.81 (d, J=8.0 Hz, 1H), 7.11-7.06 (m, 2H), 6.83 (d, J=8.4 Hz, 1H), 5.86 (dd, J=4.4, 10.4 Hz, 1H), 4.50 (dd, J=10.4, 14.0 Hz, 1H), 4.10 (q, J=7.2 Hz, 2H), 3.87-3.84 (m, 3H), 3.70 (dd, J=4.4, 14.4 Hz, 1H), 2.89-2.84 (m, 3H), 2.44 (t, J=7.2 Hz, 2H), 1.62-1.56 (m, 2H), 1.49-1.41 (m, 5H), 0.97-0.91 (m, 3H).

Synthesis of compound 1-7: The compound 1-6 (370.00 mg, 700.00 μmol, 1 eq) was dissolved in methanol (10 mL), Pd/C (100 mg, 10% purity) was added under nitrogen atmosphere, and the mixture was replaced with hydrogen for 3 times under vacuum condition, and stirred at 60° C. under hydrogen atmosphere (50 Psi) for 16 h. The reaction solution was filtered with diatomite to remove solids, and the filter cake was washed with EtOAc, concentrated, and purified by using prep-HPLC (a formic acid system) to obtain a compound 1-7 (128 mg, yield: 36.38%) and a compound 1-7A (25 mg, yield: 7.13%).

¹H-NMR(400 MHz, CDCl₃) of the compound 1-7: δ=7.25 (br s, 1H), 7.15-7.07 (m, 3H), 6.82 (d, J=8.0 Hz, 1H), 5.83 (dd, J=5.2, 9.6 Hz, 1H), 5.28 (s, 2H), 4.52 (dd, J=9.2, 14.4 Hz, 1H), 4.10 (q, J=7.2 Hz, 2H), 3.84 (s, 3H), 3.79 (dd, J=5.2, 14.4 Hz, 1H), 2.80 (s, 3H), 2.51 (t, J=8.0 Hz, 2H), 1.64-1.58 (m, 2H), 1.46 (t, J=7.2 Hz, 3H), 1.35-1.29 (m, 6H), 0.90-0.87 (m, 3H).

¹H-NMR(400 MHz, CDCl₃) of the compound 1-7A: δ=7.43 (d, J=7.2 Hz, 1H), 7.16-7.10 (m, 3H), 6.84 (d, J=8.4 Hz, 1H), 6.36-6.30 (m, 1H), 6.26-6.18 (m, 1H), 5.85 (dd, J=5.2, 9.6 Hz, 1H), 5.36 (s, 2H), 4.52 (dd, J=9.6, 14.4 Hz, 1H), 4.12 (q, J=7.2 Hz, 2H), 3.85 (s, 3H), 3.80 (dd, J=4.8, 14.4 Hz, 1H), 2.81 (s, 3H), 2.30-2.23 (m, 2H), 1.51-1.447 (m, 5H), 1.43-1.35 (m, 2H), 0.97-0.92 (m, 3H).

Synthesis of Example 1:

The compound 1-7 (40 mg, 79.58 μmol, 1 eq) and chlorobutyryl chloride (11.22 mg, 79.58 μmol, 8.91 μL, 1 eq) were dissolved in DCE (2 mL), DIEA (10.29 mg, 79.58 μmol, 13.86 μL, 1 eq) was added to the reaction solution, and the mixture was stirred at 50° C. for 3 h. The reaction solution was concentrated and purified by using prep-HPLC (a formic acid system) to obtain a white solid of Example 1 (14.04 mg, yield: 29.06%).

¹H-NMR (400 MHz, CDCl₃) δ=7.94 (br s, 1H), 7.65-7.57 (m, 2H), 7.07 (d, J=2.0 Hz, 1H), 7.09 (s, 1H), 6.83 (d, J=8.4 Hz, 1H), 5.85 (dd, J=4.4, 10.0 Hz, 1H), 4.51 (dd, J=10.4, 14.4 Hz, 1H), 4.10 (q, J=6.8 Hz, 2H), 3.85 (s, 3H), 3.74 (dd, J=4.4, 14.4 Hz, 1H), 3.69 (t, J=6.4 Hz, 2H), 2.84 (s, 3H), 2.74-2.60 (m, 4H), 2.24 (quin, J=6.8 Hz, 2H), 1.63-1.56 (m, 2H), 1.46 (t, J=7.2 Hz, 3H), 1.28 (br s, 6H), 0.92-0.82 (m, 3H).

LCMS: 607.0([M+H]+).

### EXAMPLE 2

### (S)-2-[1-(3-ethoxy-4-methoxyphenyl)-2-alkylsulfonyl ethyl]-4-(γ-chlorobutyramido)-5-hexene (−1) yl isoindoline-1, 3-diketone

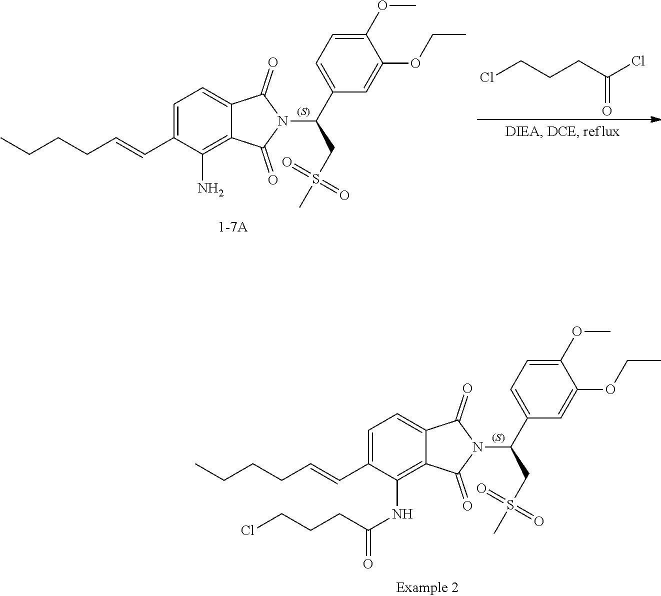

The compound 1-7A (20.00 mg, 39.95 μmol, 1 eq) and chlorobutyryl chloride (6.20 mg, 43.95 μmol, 4.92 μL, 1.1 eq) were dissolved in DCE (2 mL), DIEA (5.16 mg, 39.95 μmol, 6.96 μL, 1 eq) was added to the reaction solution, and the mixture was stirred at 50° C. for 16 h. The mixture was concentrated in reduced pressure and the crude product was purified by using prep-HPLC (a formic acid system) to obtain a white solid of Example 2 (5.13 mg, yield: 21.22%).

¹H-NMR (400 MHz, CDCl3) δ=8.03 (br s, 1H), 7.83 (d, J=7.6 Hz, 1H), 7.62 (d, J=7.6 Hz, 1H), 7.11-7.06 (m, 2H), 6.83 (d, J=8.8 Hz, 1H), 6.42-6.28 (m, 2H), 5.85 (dd, J=4.4, 10.4 Hz, 1H), 4.51 (br dd, J=10.4, 14.4 Hz, 1H), 4.10 (q, J=6.8 Hz, 2H), 3.85 (s, 3H), 3.77-3.62 (m, 3H), 2.83 (s, 3H), 2.69 (br s, 2H), 2.23 (quin, J=7.2 Hz, 4H), 1.50-1.43 (m, 5H), 1.40-1.33 (m, 2H), 0.96-0.87 (m, 3H).

LCMS: 605.1([M+H]+).

### EXAMPLE 3

### (S)-2-[1-(3-ethoxy-4-methoxyphenyl)-2-alkylsulfonyl ethyl]-4-cyclopropanamido-5-octyl isoindoline-1, 3-diketone

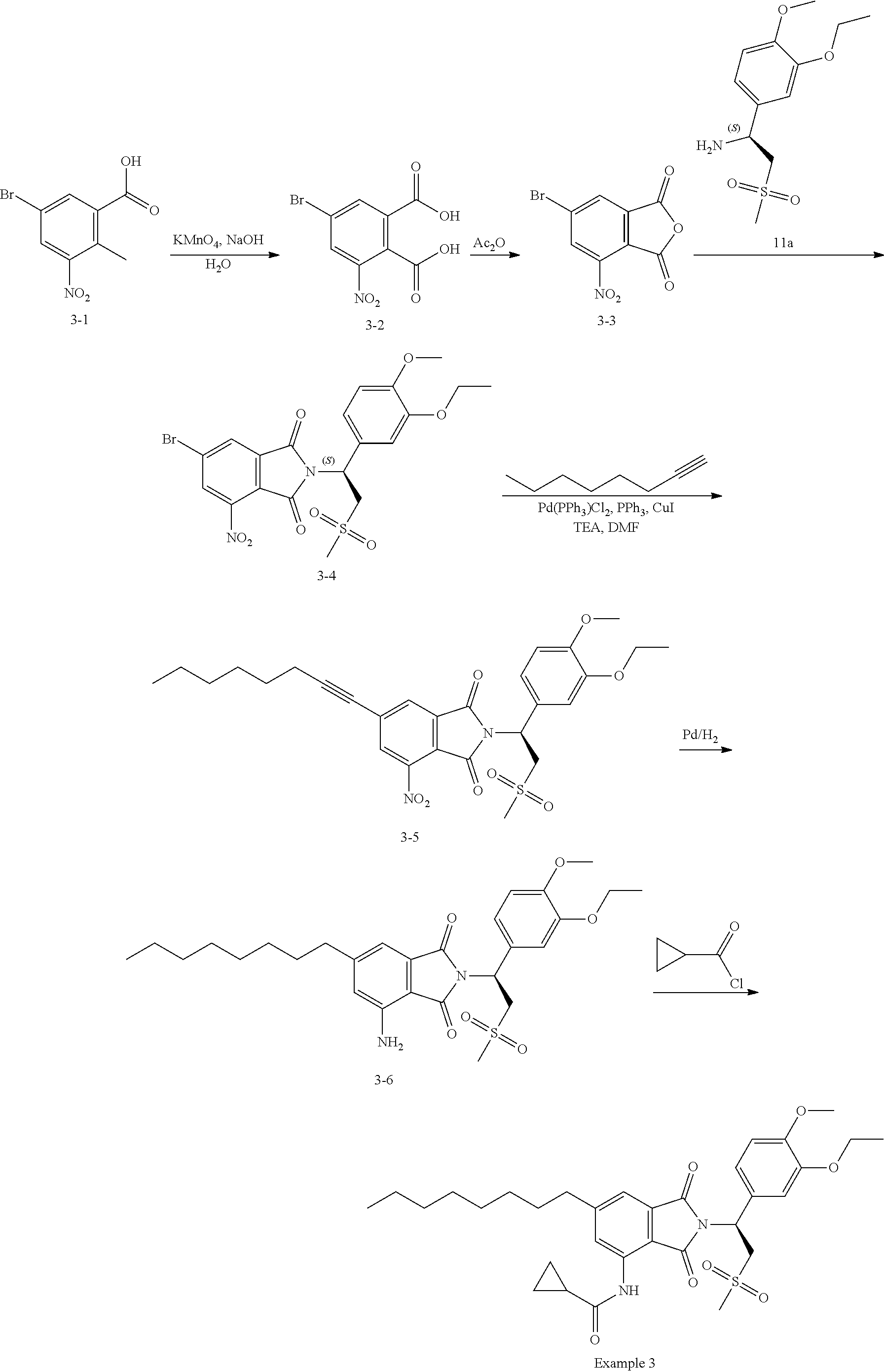

Synthesis of compound 3-2: A compound 3-1 5-bromo-2-methyl-3-nitrobenzoic acid (20 g, 76.91 mmol, 1 eq.) was dissolved in H₂O (20 mL), NaOH (9.23 g, 230.73 gmmol, 3 eq) was added into the obtained solution, the mixture was heated up to 80° C., and KMnO4 (97.24 g, 615.29 mmol, 8 eq) was added in batches within 3 h; and after the addition was finished, stirring was continued for 30 min at 80° C., the reaction mixture was filtered and the filter cake was washed with hot water (300 mL*3). The aqueous phase was cooled with ice water, and the pH was adjusted to 1 with 2M HCl. The product was extracted with EtOAc (400 mL*3), washed with a saturated saline solution after combination of organic phases, dried with Na₂SO₄, filtered, and concentrated to obtain a compound 3-2 (5 g, yield: 22.42%) as a yellow solid.

¹H-NMR (400 MHz, DMSO-d6) δ=13.98 (br s, 2H), 8.52 (d, J=2.0 Hz, 1H), 8.33 (d, J=2.0 Hz, 1H).

Synthesis of compound 3-3: The compound 3-2 (5 g, 17.24 mmol, 1 eq) was dissolved in Ac₂O (20 mL), and stirred at 140° C. for 16 h. The reaction mixture was concentrated to obtain a crude product, and the crude product was purified by silica gel chromatography (petroleum ether: ethyl acetate=100/0 to 1/1) to obtain a compound 3-3 (4.5 g, crude) as a yellow solid.

Synthesis of compound 3-4: The compound 3-3 (4 g, 14.71 mmol, 1 eq) and a compound 11a (4.02 g, 14.71 mmol, 1 eq.) were dissolved in AcOH (80 mL), and stirred at 120° C. for 16 h. The reaction mixture was concentrated to obtain a crude product, and the crude product was purified by silica gel chromatography (petroleum ether:ethyl acetate=100/0 to 1/1) to obtain a compound 3-4 (2.9 g, yield: 37.40%) as a yellow solid.

¹H-NMR (400 MHz, CDCl₃) δ=8.24 (d, J=1.6 Hz, 1H), 8.21 (d, J=1.6 Hz, 1H), 7.13-7.06 (m, 2H), 6.84 (d, J=7.6 Hz, 1H), 5.91 (dd, J=11.2, 4.4 Hz, 1H), 4.57 (dd, J=14.4, 11.2 Hz, 1H), 4.14-4.07 (m, 2H), 3.85 (s, 3H), 3.66 (dd, J=14.4, 4.0 Hz, 1H), 2.91 (s, 3H), 1.47 (t, J=7.2 Hz, 3H).

Synthesis of compound 3-5: The compound 3-4 (400.00 mg, 758.52 μmol, 1 eq), PdCl₂(PPh3)2 (106.48 mg, 151.70 μmol, 0.2 eq), CuI (28.89 mg, 151.70 μmol, 0.2 eq), DIEA (294.09 mg, 2.28 mmol, 396.35 μL, 3 eq) and 1-octyne (417.93 mg, 3.79 mmol, 5 eq) were dissolved in DMF (4 mL), the reaction solution was stirred at 60° C. under N₂ atmosphere for 16 h, and water was (10 mL) added to the reaction system. The product was extracted with ethyl acetate (10 mL*3), washed with a saturated saline solution after combination of organic phases, dried with Na₂SO₄, filtered, and concentrated to obtain a crude product; and the crude product was purified by silica gel chromatography (petroleum ether:ethyl acetate=100:1 to 1:1) to obtain a compound 3-5 (130 mg, yield: 30.79%) as a yellow solid.

¹H-NMR (400 MHz CDCl₃) δ=7.13-7.07 (m, 3H), 6.85-6.80 (m, 2H), 5.82 (dd, J=9.6, 5.2 Hz, 1H), 5.15 (s, 2H), 4.49 (dd, J=14.4, 9.6 Hz, 1H), 4.10 (d, J=7.2 Hz, 2H), 3.84 (s, 3H), 3.78 (dd, J=14.4, 5.2 Hz, 1H), 2.80 (s, 3H), 2.39 (t, J=7.2 Hz, 2H), 1.63-1.56 (m, 2H), 1.46 (t, J=7.2 Hz, 3H), 1.32-1.30 (m, 3H), 0.88-0.87 (m, 4H).

Synthesis of compound 3-6: The compound 3-5 (130 mg, 233.55 μmol, 1 eq) was dissolved in EtOAc (15 mL), Pd/C (140 mg, 10%) was added under nitrogen atmosphere, and the mixture was replaced with hydrogen for 3 times under vacuum condition, and stirred at 60° C. under hydrogen atmosphere (50 Psi) for 16 h. The reaction solution was filtered with diatomite to remove solids, the filter cake was washed with EtOAc, and the filtrate was concentrated to obtain a compound 3-6 (100 mg, yield: 80.68%) as a yellow solid.

¹H-NMR (400 MHz CDCl₃) δ=7.14-7.08 (m, 2H), 7.00-6.96 (m, 1H), 6.82 (d, J=8.4 Hz, 1H), 6.62 (s, 1H), 5.82 (dd, J=5.2, 9.6 Hz, 1H), 5.30 (s, 1H), 5.12 (s, 2H), 4.51 (dd, J=14.8, 9.6 Hz, 1H), 4.11 (q, J=7.2 Hz, 2H), 3.84 (s, 3H), 3.79 (dd, J=14.8, 5.2 Hz, 1H), 2.80 (s, 3H), 2.57 (t, J=7.6 Hz, 2H), 2.05-1.98 (m, 1H), 1.57 (br s, 2H), 1.46 (t, J=7.2 Hz, 3H), 1.26 (br d, J=6.8 Hz, 8H), 0.89-0.86 (m, 3H).

Synthesis of Example 3:

The compound 3-6 (10 mg, 18.84 μmol, 1 eq) and cyclopropionyl chloride (9.85 mg, 94.22 μmol, 8.56 μL, 5 eq) were dissolved in DCE (1 mL), and DIEA (19.48 mg, 150.75 μmol, 26.26 μL, 8 eq) was added into the reaction solution, and stirred at 90° C. for 2 h. The reaction mixture was concentrated, and the crude product was purified by using prep-HPLC (a formic acid system) to obtain a white solid of Example 3 (8.2 mg, yield: 72.68%).

¹H-NMR (400 MHz, CDCl₃) δ=9.60 (s, 1H), 8.58 (s, 1H), 7.30 (s, 1H), 7.12-7.08 (m, 2H), 6.86-6.82 (m, 1H), 5.86 (dd, J=10.4, 4.4 Hz, 1H), 4.55 (dd, J=14.4, 10.4 Hz, 1H), 4.11 (q, J=6.8 Hz, 2H), 3.85 (s, 3H), 3.74 (dd, J=14.4, 4.4 Hz, 1H), 2.86 (s, 3H), 2.69-2.64 (m, 2H), 1.67-1.58 (m, 3H), 1.47 (t, J=7.2 Hz, 3H), 1.26 (br d, J=12.4 Hz, 10H), 1.12 (quin, J=3.6 Hz, 2H), 0.97-0.91 (m, 2H), 0.89-0.84 (m, 3H).

LCMS: 599.1 [M+H]+.

### EXAMPLE 4

### (S)-2-[1-(3-ethoxy-4-methoxyphenyl)-2-alkylsulfonyl ethyl]-4-acetamidomethyl-7-pentyl isoindoline-1, 3-diketone

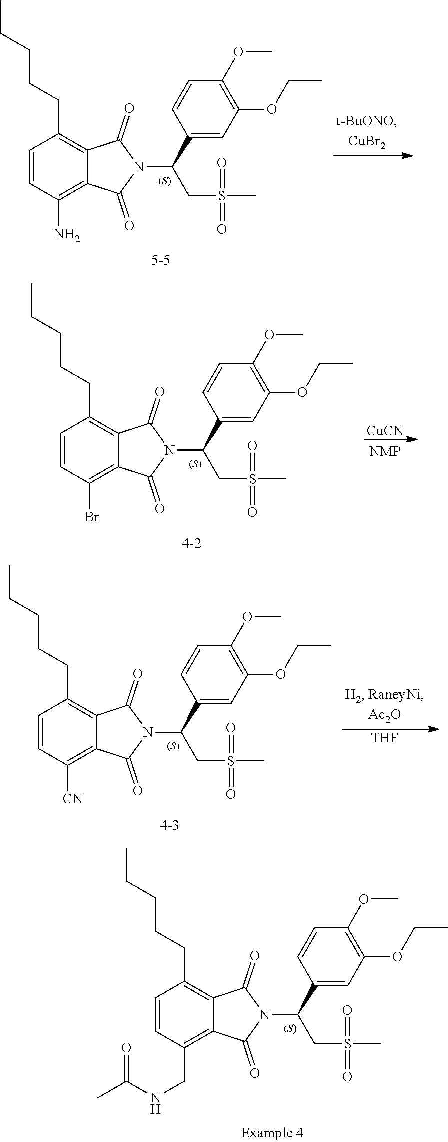

Example 4 was synthesized via synthetic route 6.

LCMS: 545.1 ([M+H]+).

### EXAMPLE 5

### (S)-2-[1-(3-ethoxy-4-methoxyphenyl)-2-alkylsulfonyl ethyl]-4-(γ-chlorobutyramido)-7-pentyl isoindoline-1, 3-diketone

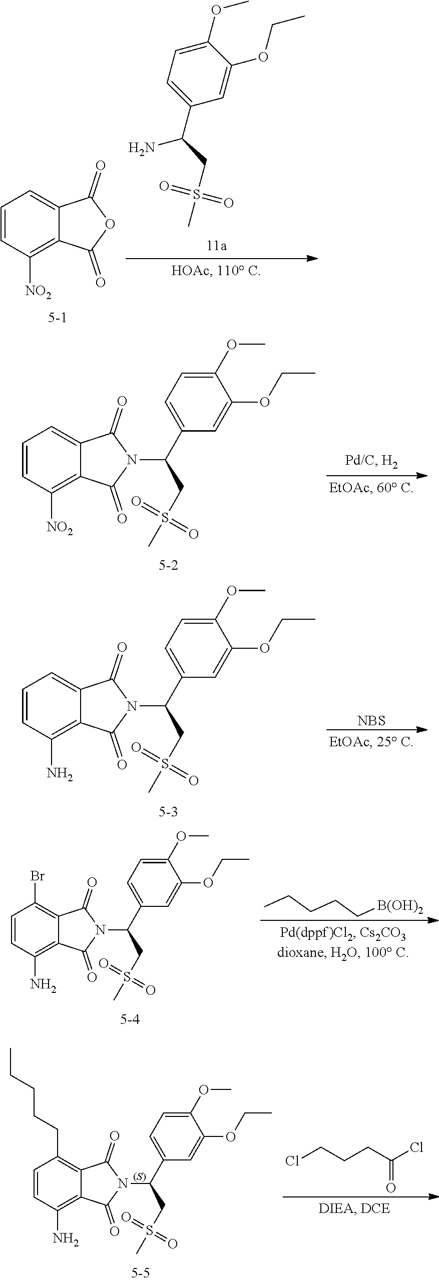

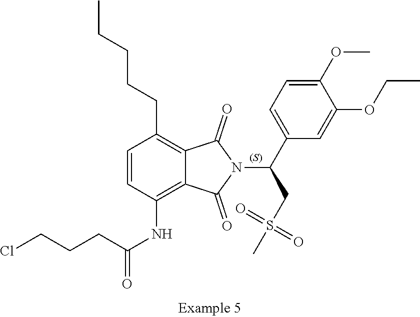

Synthesis of compound 5-2: A compound 5-1 (21.19 g, 109.75 mmol, 1 eq) and the compound 11a (30 g, 109.75 mmol, 1 eq) were dissolved in HOAc (500 mL), and stirred at 120° C. for 16 h. The reaction mixture was concentrated to obtain a crude product, and the crude product was purified by silica gel chromatography (petroleum ether:ethyl acetate=100/0 to 1/1) to obtain a compound 5-2 (43.3 g, yield: 89.98%) as a yellow solid.

¹H-NMR (400 MHz, CDCl₃) δ=8.13-8.07 (m, 2H), 7.92-7.86 (m, 1H), 7.15-7.09 (m, 2H), 6.84 (d, J=8.0 Hz, 1H), 5.93 (dd, J=4.0, 10.8 Hz, 1H), 4.58 (dd, J=10.8, 14.4 Hz, 1H), 4.15-4.07 (m, 2H), 3.85 (s, 3H), 3.70 (dd, J=4.4, 14.4 Hz, 1H), 2.90 (s, 3H), 1.47 (t, J=7.2 Hz, 3H).

Synthesis of compound 5-3: The compound 5-2 (50.00 g, 111.15 mmol, 1 eq) was dissolved in EtOAc (400 mL), Pd/C (9 g, 10%) was added under nitrogen atmosphere, and the mixture was replaced with hydrogen for 3 times under vacuum condition, and stirred at 60° C. under hydrogen atmosphere (50 Psi) for 12 h. The reaction solution was filtered with diatomite to remove solids, the filter cake was washed with EtOAc, and the filtrate was concentrated to obtain a compound 5-3 (4 g, yield: 85.73%) as a yellow solid.

¹H-NMR (400 MHz, CDCl₃) δ=7.37 (dd, J=7.2, 8.4 Hz, 1H), 7.13-7.10 (m, 2H), 7.10-7.08 (m, 1H), 6.83-6.78 (m, 2H), 5.83 (dd, J=4.8, 9.8 Hz, 1H), 5.20 (s, 2H), 4.54-4.47 (m, 1H), 4.12-4.06 (m, 2H), 3.83 (s, 3H), 3.78 (dd, J=5.2, 14.8 Hz, 1H), 2.79 (s, 3H), 1.47-1.42 (m, 3H).

Synthesis of compound 5-4: The compound 5-3 (47.4 g, 113.27 mmol, 1 eq) was dissolved in ethyl acetate (500 mL), added with NBS (20.16 g, 113.27 mmol, 1 eq), and stirred at 25° C. for 16 h. The reaction solution was concentrated to obtain a crude product, and the crude product was purified by silica gel chromatography (petroleum ether:ethyl acetate=100/0 to 1/1) to obtain a compound 5-4 (26.52 g, yield: 42.07%) as a yellow solid.

¹H-NMR (400 MHz, CDCl₃) δ=7.44 (d, J=8.4 Hz, 1H), 7.16-7.08 (m, 2H), 6.84 (d, J=8.0 Hz, 1H), 6.71 (d, J=8.4 Hz, 1H), 5.86 (dd, J=4.4, 10.4 Hz, 1H), 5.55-5.15 (m, 2H), 4.55 (dd, J=10.4, 14.4 Hz, 1H), 4.15-4.02 (m, 2H), 3.86 (s, 3H), 3.77 (dd, J=4.8, 14.4 Hz, 1H), 2.85 (s, 3H), 1.47 (t, J=7.2 Hz, 3H).

Synthesis of compound 5-5: The compound 5-4 (13.98 g, 28.11 mmol, 1 eq), Cs₂CO₃ (27.47 g, 84.33 mmol, 3 eq), and amyl boric acid (6.52 g, 56.22 mmol, 2 eq) were dissolved in dioxane (150 mL) and water (30 mL), Pd(dppf)Cl₂ (4.11 g, 5.62 mmol, 0.2 eq) was added under N₂ atmosphere, the mixture was stirred at 60° C. under N₂ atmosphere for 18 h, the reaction solution was concentrated, water (50 mL) and ethyl acetate (50 mL) were added into the reaction solution, extraction was performed with ethyl acetate (50 mL*3), the combined organic layers were washed with a saturated saline solution, dried with Na₂SO₄, filtered, and concentrated to obtain a crude product, and the crude product was purified by silica gel chromatography (SiO₂, petroleum ether:ethyl acetate=100/0 to 1/1) to obtain a compound 5-5 (4.78 g, yield: 34.80%) as a yellow solid.

¹H-NMR (400 MHz, CDCl₃) δ=7.18 (d, J=8.4 Hz, 1H), 7.16-7.10 (m, 2H), 6.83 (d, J=8.4 Hz, 1H), 6.75 (d, J=8.4 Hz, 1H), 5.83 (dd, J=5.2, 9.6 Hz, 1H), 5.14 (s, 2H), 4.50 (dd, J=9.6, 14.4 Hz, 1H), 4.16-4.09 (m, 2H), 3.85 (s, 3H), 3.83-3.78 (m, 1H), 3.64 (t, J=6.8 Hz, 1H), 2.96-2.86 (m, 2H), 2.83-2.76 (m, 3H), 1.62-1.57 (m, 2H), 1.46 (t, J=7.2 Hz, 3H), 1.38-1.27 (m, 4H), 0.94-0.85 (m, 3H).

Synthesis of Example 5:

The compound 5-5 (4.04 g, 8.27 mmol, 1 eq) and chlorobutyryl acid chloride (2.33 g, 16.54 mmol, 1.85 mL, 2 eq) were dissolved in DCE (80 mL), DIEA (4.27 g, 33.07 mmol, 5.76 mL, 4 eq) was added to the reaction solution, and the mixture was stirred at 90° C. for 3 h. The reaction solution was concentrated, a saturated NaHCO₃ (20 mL) aqueous solution and DCM (20 mL) were added, extraction was performed with dichloromethane (3*20 mL), the combined organic layers were washed with a saturated saline solution, dried with Na₂SO₄, filtered, and concentrated to obtain a crude product, and the crude product was purified by silica gel chromatography (SiO₂, petroleum ether:ethyl acetate=1/0 to 1/1) to obtain a yellow solid as Example 5 (4.12 g, ee value: 96.7%, yield: 84.01%).

¹H-NMR (400 MHz, CDCl₃) δ=9.58 (s, 1H), 8.63 (d, J=8.8 Hz, 1H), 7.43 (d, J=8.8 Hz, 1H), 7.13-7.08 (m, 2H), 6.88-6.82 (m, 1H), 5.86 (dd, J=4.8, 10.0 Hz, 1H), 4.53 (dd, J=10.4, 14.4 Hz, 1H), 4.12 (q, J=7.2 Hz, 2H), 3.85 (s, 3H), 3.75 (dd, J=4.8, 14.4 Hz, 1H), 3.66 (t, J=6.4 Hz, 2H), 3.03-2.94 (m, 2H), 2.85 (s, 3H), 2.66 (t, J=7.2 Hz, 2H), 2.22 (quin, J=6.8 Hz, 2H), 1.65 -1.57 (m, 2H), 1.47 (t, J=7.2 Hz, 3H), 1.36-1.30 (m, 4H), 0.91-0.86 (m, 3H).

LCMS: 593.1([M+H]+).

### EXAMPLE 6

### (S)-2-[1-(3-ethoxy-4-methoxyphenyl)-2-alkylsulfonyl ethyl]-4-cyclopropanamido-7-pentyl isoindoline-1, 3-diketone

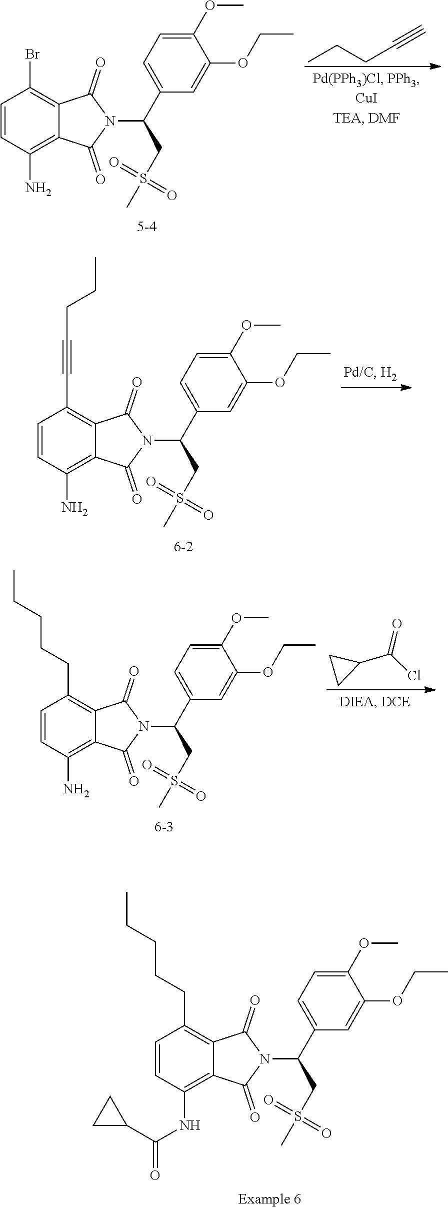

Synthesis of compound 6-2:

The compound 5-4 (200 mg, 402.12 μmol, 1 eq), PdCl2(PPh3)2 (56.45 mg, 80.42 μmol, 0.2 eq), CuI (15.32 mg, 80.42 μmol, 0.2 eq), DIEA (155.91 mg, 1.21 mmol, 210.12 μL, 3 eq), and 1-pentylene (273.91 mg, 4.02 mmol, 394.69 μL, 10 eq) were dissolved in DMF(2 mL), the reaction solution was stirred at 60° C. under N₂ atmosphere for 16 h, water (5 mL) was added into the reaction system, extraction was performed with ethyl acetate (50 mL*3), the combined organic layers were washed with a saturated saline solution, dried with Na₂SO₄, filtered, and concentrated to obtain a crude product, and the crude product was purified by silica gel chromatography (SiO₂, petroleum ether:ethyl acetate=1/0 to 1/1) to obtain a compound 6-2 (S)-2-[1-(3-ethoxy-4-methoxyphenyl)-2-methylsulfonyl ethyl]-4-amino-6-[pentyl-1-alkynyl] isoindoline-1, 3-diketone (98 mg, yield: 50.29%) as a yellow solid.

¹H-NMR (400 MHz CDCl₃) δ=7.36 (d, J=8.4 Hz, 1H), 7.15-7.10 (m, 2H), 6.82 (d, J=8.8 Hz, 1H), 6.74 (d, J=8.4 Hz, 1H), 5.84 (dd, J=9.6, 5.2 Hz, 1H), 5.32 (d, J=13.2 Hz, 2H), 4.48 (dd, J=14.4, 9.2 Hz, 1H), 4.14-4.08 (m, 2H), 3.84 (s, 3H), 3.84-3.79 (m, 1H), 2.81-2.77 (m, 3H), 2.46 (t, J=7.2 Hz, 2H), 1.67 (sxt, J=7.2 Hz, 2H), 1.46 (t, J=7.2 Hz, 3H), 1.08 (t, J=7.2 Hz, 3H).

Synthesis of compound 6-3:

The compound 6-2 (98 mg, 202.24 μmol, 1 eq) was dissolved in EtOAc (10 mL), Pd/C (100 mg, 10%) was added under nitrogen atmosphere, and the mixture was replaced with hydrogen for 3 times under vacuum condition, and stirred at 60° C. under hydrogen atmosphere (50 Psi) for 16 h. The reaction solution was filtered with diatomite to remove solids, the filter cake was washed with EtOAc, and the filtrate was concentrated to obtain a compound 6-3 (60 mg, yield: 60.72%) as a yellow solid.

¹H-NMR (400 MHz CDCl₃) δ=7.20-7.10 (m, 3H), 6.83 (d, J=8.4 Hz, 1H), 6.75 (d, J=8.4 Hz, 1H), 5.84 (dd, J=9.2, 5.2Hz, 1H), 5.14 (s, 2H), 4.50 (dd, J=14.8, 9.6 Hz, 1H), 4.11 (q, J=6.8 Hz, 2H), 3.85-3.84 (m, 3H), 3.84-3.79 (m, 1H), 2.93-2.88 (m, 2H), 2.78 (s, 3H), 1.62-1.55 (m, 2H), 1.46 (t, J=7.2 Hz, 3H), 1.35-1.28 (m, 4H), 0.91-0.85 (m, 3H).

Synthesis of Example 6:

The compound 6-3 (12.5 mg, 25.58 μmol, 1 eq) and cyclopropionyl chloride (13.37 mg, 127.92 μmol, 11.63 μL, 5 eq) were dissolved in DCE (1 mL), and DIEA (26.45 mg, 204.67 μmol, 35.65 μL, 8 eq) was added and stirred at 90° C. for 2 h. The reaction solution was concentrated and purified by using prep-HPLC (a formic acid system) to obtain a white solid as Example 6 (5 mg, yield: 35.11%).

¹H-NMR (400 MHz, CDCl₃) δ=9.76 (s, 1H), 8.63 (d, J=8.8 Hz, 1H), 7.41 (d, J=8.8 Hz, 1H), 7.14-7.08 (m, 2H), 6.88-6.81 (m, 1H), 5.87 (dd, J=10.0, 4.8 Hz, 1H), 4.53 (dd, J=14.4, 10.0 Hz, 1H), 4.12 (q, J=7.2 Hz, 2H), 3.85 (s, 3H), 3.76 (dd, J=14.4, 4.4 Hz, 1H), 3.01-2.94 (m, 2H), 2.84 (s, 3H), 1.67-1.58 (m, 3H), 1.47 (t, J=7.2 Hz, 3H), 1.37-1.27 (m, 4H), 1.15-1.07 (m, 2H), 0.96-0.84 (m, 5H).

LCMS: 557.1 ([M+H]+).

### EXAMPLE 7

### (S)-2-[1-(3-ethoxy-4-methoxyphenyl)-2-alkylsulfonyl ethyl]-4-acetamido-7-pentenyl (−1) isoindoline-1, 3-diketone

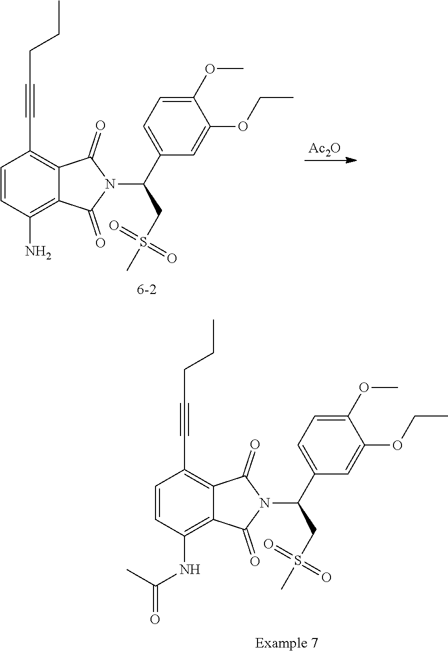

Synthesis of Example 7:

The compound 6-2 (180 mg, 371.47 μmol, 1.0 eq) was dissolved in Ac₂O (1 mL), the reaction solution was stirred for 3 h, and then the reaction solution was concentrated and purified by using prep-HPLC (a formic acid system) to obtain a white solid as Example 7 (119 mg, yield: 60.8%).

¹H-NMR (400 MHz DMSO-δ₆) δ=9.77 (s, 1H), 8.46 (d, J=8.7 Hz, 1H), 7.74 (d, J=8.7 Hz, 1H), 7.08 (d, J=1.9 Hz, 1H), 7.03-6.93 (m, 2H), 5.79 (dd, J=10.4, 4.3 Hz, 1H), 4.35 (dd, J=14.3, 10.5 Hz, 1H), 4.17 (dd, J=14.3, 4.4 Hz, 1H), 4.04 (d, J=7.0 Hz, 2H), 3.75 (s, 3H), 3.04 (s, 3H), 2.49(d, J=6.9Hz, 2H), 2.22 (s, 3H), 1.62 (p, J=7.2 Hz, 2H), 1.34 (t, J=7.0 Hz, 3H), 1.06 (t, J=7.4 Hz, 3H).

LCMS :527.2 ([M+H]+).

### EXAMPLE 8

### (S)-2-[1-(3-ethoxy-4-methoxyphenyl)-2-alkylsulfonyl ethyl]-4-decanamido-7-pentenyl (−1) isoindoline-1, 3-diketone

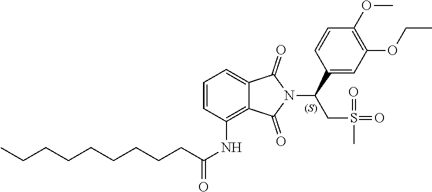

Example 8 was synthesized via synthetic route 3.

¹H-NMR (400 MHz, CDCl₃) δ=9.47 (s, 1H), 8.79 (d, J=8.4 Hz, 1H), 7.74-7.59 (m, 1H), 7.48 (d, J=7.2 Hz, 1H), 7.11 (dd, J=5.9, 2.1 Hz, 2H), 6.84 (d, J=8.9 Hz, 1H), 5.87 (dd, J=10.4 Hz, 4.4Hz, 1H), 4.62-4.47 (m, 1H), 4.17-4.05 (m, 2H), 3.85 (s, 3H), 3.73 (s, 1H), 2.86 (s, 3H), 2.46 (d, J=7.5 Hz, 2H), 1.84-1.69 (m, 2H), 1.50-1.44 (m, 3H), 1.43-1.17 (m, 12H), 0.94-0.75 (m, 3H).

LCMS: 573.6 ([M+H]+).

### EXAMPLE 9

### (S)-2-[1-(3-ethoxy-4-methoxyphenyl)-2-alkylsulfonyl ethyl]-4-acetamido-7-pentyl isoindoline-1, 3-diketone

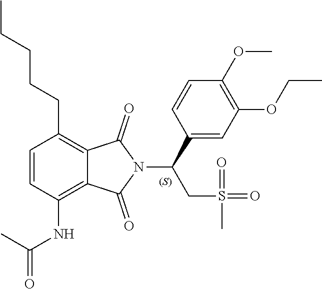

Example 9 was synthesized via synthetic route 3.

¹H-NMR (400 MHz DMSO-δ₆) δ=9.70 (s, 1H), 8.35 (d, J=8.6 Hz, 1H), 7.62 (d, J=8.6 Hz, 1H), 7.09 (d, J=1.8 Hz, 1H), 7.03-6.92 (m, 2H), 5.78 (dd, J=10.4, 4.3 Hz, 1H), 4.37 (dd, J=14.3 Hz, 10.5Hz, 1H), 4.15 (dd, J=14.3 Hz, 4.4Hz, 1H), 4.03 (q, J=7.0 Hz, 2H), 3.75 (s, 3H), 3.02 (s, 3H), 2.99-2.92 (m, 2H), 2.19 (s, 3H), 1.57(p, J=7.3 Hz, 2H), 1.38-1.25 (m, 7H), 0.87 (t, J=6.9 Hz, 3H).

LCMS: 531.2 ([M+H]+).

### EXAMPLE 10

### (S)-2-[1-(3-ethoxy-4-methoxyphenyl)-2-alkylsulfonyl ethyl]-4-butylamido-7-pentyl isoindoline-1, 3-diketone

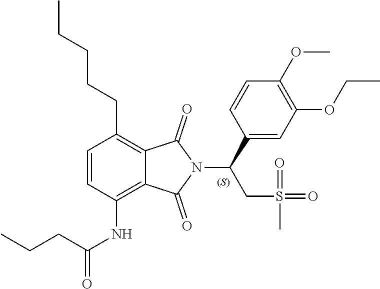

Example 10 was synthesized via synthetic route 3.

¹H-NMR (400 MHz, CDCl₃) δ=9.54 (s, 1H), 8.66 (d, J=8.4 Hz, 1H), 7.42 (d, J=8.4 Hz, 1H), 7.15-7.06 (m, 2H), 6.84 (d, J=8.8 Hz, 1H), 5.85 (dd, J=4.4, 10.0 Hz, 1H), 4.52 (dd, J=10.4, 14.4 Hz, 1H), 4.11 (q, J=6.8 Hz, 2H), 3.85 (s, 3H), 3.76 (dd, J=4.4, 14.4 Hz, 1H), 3.03 - 2.94 (m, 2H), 2.84 (s, 3H), 2.42 (t, J=7.6 Hz, 2H), 1.78 (qd, J=7.2, 14.8 Hz, 2H), 1.62-1.57 (m, 2H), 1.47 (t, J=7.2 Hz, 3H), 1.37-1.28 (m, 4H), 1.02 (t, J=7.2 Hz, 3H), 0.94-0.82 (m, 3H).

LCMS: 559.1([M+H]+).

### EXAMPLE 11

### (S)-2-[1-(3-ethoxy-4-methoxyphenyl)-2-alkylsulfonyl ethyl]-4-isovaleramido-7-butyl isoindoline-1, 3-diketone

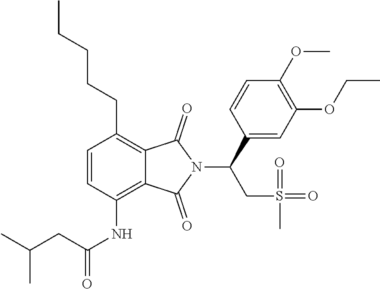 [SMILES: CCCCCc1ccc(NC(=O)CC(C)C)c2c1C(=O)N([C@H](CS(C)(=O)=O)c1ccc(OC)c(OCC)c1)C2=O]

¹H-NMR (400 MHz, CDCl₃) δ=9.52 (s, 1H), 8.67 (d, J=8.4 Hz, 1H), 7.42 (d, J=8.8 Hz, 1H), 7.14-7.08 (m, 2H), 6.85 (d, J=8.4 Hz, 1H), 5.85 (dd, J=4.8, 10.0 Hz, 1H), 4.52 (dd, J=10.0, 14.4 Hz, 1H), 4.11 (q, J=7.2 Hz, 2H), 3.85 (s, 3H), 3.77 (dd, J=4.8, 14.4 Hz, 1H), 3.03-2.93 (m, 2H), 2.83 (s, 3H), 2.34-2.28 (m, 2H), 2.28-2.19 (m, 1H), 1.64-1.58 (m, 2H), 1.47 (t, J=7.2 Hz, 3H), 1.37-1.29 (m, 4H), 1.03 (d, J=6.4 Hz, 6H), 0.92-0.85 (m, 3H). LCMS: 573.1 ([M+H]+).

### EXAMPLE 12

### (S)-2-[1-(3-ethoxy-4-methoxyphenyl)-2-alkylsulfonyl ethyl]-4-caproamido-7-butyl isoindoline-1, 3-diketone

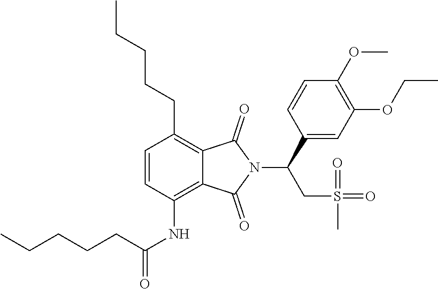 [SMILES: CCCCCC(=O)Nc1ccc(CCCCC)c2c1C(=O)N([C@H](CS(C)(=O)=O)c1ccc(OC)c(OCC)c1)C2=O]

¹H-NMR (400 MHz, CDCl₃) δ=9.54 (br s, 1H), 8.66 (br d, J=8.4 Hz, 1H), 7.42 (br d, J=8.8 Hz, 1H), 7.19-7.03 (m, 2H), 6.84 (br d, J=8.4 Hz, 1H), 5.85 (br dd, J=4.0, 9.2 Hz, 1H), 4.58-4.44 (m, 1H), 4.18-4.04 (m, 2H), 3.85 (s, 3H), 3.76 (br dd, J=4.0, 14.4 Hz, 1H), 2.98 (br t, J=7.2 Hz, 2H), 2.84 (s, 3H), 2.44 (br t, J=7.2 Hz, 2H), 1.75 (br s, 2H), 1.60 (br s, 2H), 1.47 (br t, J=6.8 Hz, 3H), 1.35 (br d, J=16.4 Hz, 8H), 0.95-0.85 (m, 6H).

LCMS: 587.1 ([M+H]+).

### EXAMPLE 13

### (S)-2-[1-(3-ethoxy-4-methoxyphenyl)-2-alkylsulfonyl ethyl]-4-propanamido-7-pentyl isoindoline-1, 3-diketone

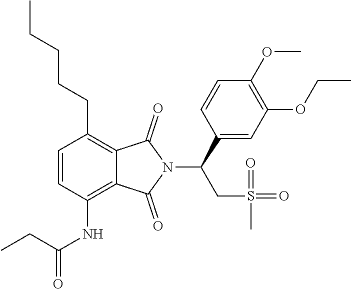 [SMILES: CCCCCc1ccc(NC(=O)CC)c2c1C(=O)N([C@H](CS(C)(=O)=O)c1ccc(OC)c(OCC)c1)C2=O]

¹H-NMR (400 MHz, CDCl₃) δ=9.56 (s, 1H), 8.66 (d, J=8.8 Hz, 1H), 7.42 (d, J=8.8 Hz, 1H), 7.15-7.06 (m, 2H), 6.84 (d, J=8.8 Hz, 1H), 5.85 (dd, J=4.8, 10.0 Hz, 1H), 4.52 (dd, J=10.0, 14.4 Hz, 1H), 4.15-4.08 (m, 2H), 3.85 (s, 3H), 3.76 (dd, J=4.8, 14.4 Hz, 1H), 3.02-2.94 (m, 2H), 2.84 (s, 3H), 2.48 (q, J=7.6 Hz, 2H), 1.63-1.58 (m, 2H), 1.47 (t, J=7.2 Hz, 3H), 1.33-1.25 (m, 7H), 0.89-0.86 (m, 3H).

LCMS: 545.1 ([M+H]+).

### EXAMPLE 14

### (S)-2-[1-(3-ethoxy-4-methoxyphenyl)-2-alkylsulfonyl ethyl]-4-acetamido-7-tridecyl isoindoline-1, 3-diketone

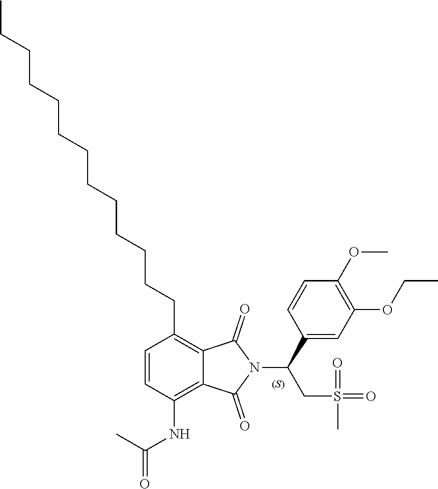

¹H-NMR (400 MHz, CDCl₃) δ=9.53 (s, 1H), 8.64 (d, J=8.8 Hz, 1H), 7.42 (d, J=8.8 Hz, 1H), 7.15-7.06 (m, 2H), 6.85 (d, J=8.8 Hz, 1H), 5.86 (dd, J=4.4, 10.0 Hz, 1H), 4.53 (dd, J=10.4, 14.4 Hz, 1H), 4.12 (q, J=7.2 Hz, 2H), 3.85 (s, 3H), 3.75 (dd, J=4.8, 14.4 Hz, 1H), 2.98 (dd, J=6.4, 8.8 Hz, 2H), 2.85 (s, 3H), 2.25 (s, 3H), 1.63-1.57 (m, 2H), 1.47 (t, J=7.2 Hz, 3H), 1.33-1.18 (m, 20H), 0.91-0.85 (m, 3H).

LCMS: 643.3 ([M+H]+).

### EXAMPLE 15

### (S)-2-[1-(3-ethoxy-4-methoxyphenyl)-2-alkylsulfonyl ethyl]-4-cyclopropanamido-7-tridecyl isoindoline-1, 3-diketone

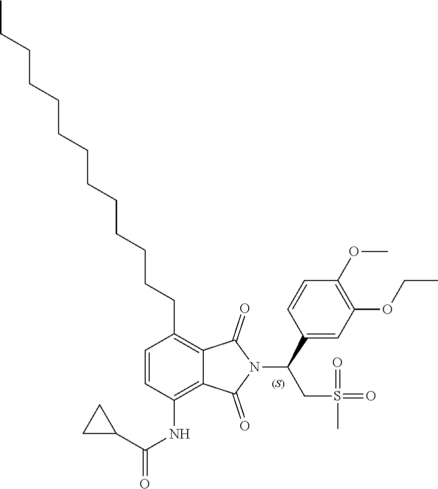

¹H-NMR (400 MHz, CDCl₃) =9.76 (s, 1H), 8.63 (d, J=8.8 Hz, 1H), 7.40 (d, J=8.8 Hz, 1H), 7.15-7.08 (m, 2H), 6.85 (d, J=8.8 Hz, 1H), 5.87 (dd, J=4.4, 10.0 Hz, 1H), 4.53 (dd, J=10.4, 14.4 Hz, 1H), 4.12 (q, J=6.8 Hz, 2H), 3.85 (s, 3H), 3.76 (dd, J=4.8, 14.4 Hz, 1H), 2.97 (br t, J=7.6 Hz, 2H), 2.84 (s, 3H), 1.68-1.57 (m, 3H), 1.47 (t, J=7.2 Hz, 3H), 1.36-1.23 (m, 20H), 1.13-1.09 (m, 2H), 0.92 (br dd, J=3.2, 7.6 Hz, 2H), 0.88 (br t, J=6.8 Hz, 3H).

LCMS: 669.2 ([M+H]+).

### EXAMPLE 16

### (S)-2-[1-(3-ethoxy-4-methoxyphenyl)-2-alkylsulfonyl ethyl]-4-butylamido-7-nonyl isoindoline-1, 3-diketone

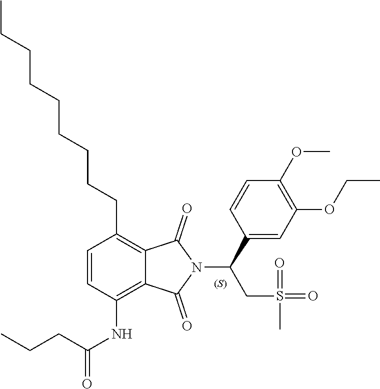

¹H-NMR (400 MHz, CDCl₃) δ=9.55 (s, 1H), 8.67 (d, J=8.8 Hz, 1H), 7.42 (d, J=8.8 Hz, 1H), 7.14-7.08 (m, 2H), 6.85 (d, J=8.8 Hz, 1H), 5.85 (dd, J=4.8, 10.0 Hz, 1H), 4.52 (dd, J=10.0, 14.4 Hz, 1H), 4.12 (q, J=7.2 Hz, 2H), 3.85 (s, 3H), 3.76 (dd, J=4.8, 14.4 Hz, 1H), 2.98 (dd, J=6.8, 8.8 Hz, 2H), 2.84 (s, 3H), 2.43 (t, J=7.6 Hz, 2H), 1.79 (sxt, J=7.6 Hz, 2H), 1.64-1.57 (m, 2H), 1.47 (t, J=7.2 Hz, 3H), 1.32-1.18 (m, 12H), 1.03 (t, J=7.6 Hz, 3H), 0.89-0.86 (m, 3H).

LCMS: 615.2 ([M+H]+).

### EXAMPLE 17

### (S)-2-[1-(3-ethoxy-4-methoxyphenyl)-2-alkylsulfonyl ethyl]-4-cyclopropanamido-7-nonyl isoindoline-1, 3-diketone

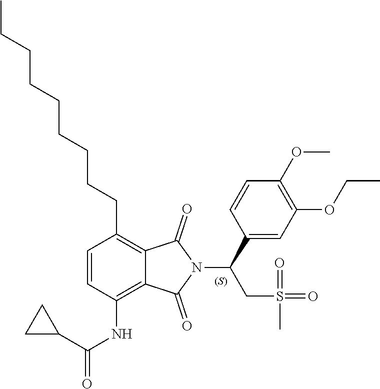

¹H-NMR (400 MHz, CDCl₃) δ=9.76 (s, 1H), 8.62 (d, J=8.8 Hz, 1H), 7.40 (d, J=8.8 Hz, 1H), 7.15-7.08 (m, 2H), 6.85 (d, J=8.8 Hz, 1H), 5.87 (dd, J=4.8, 10.0 Hz, 1H), 4.53 (dd, J=10.0, 14.4 Hz, 1H), 4.12 (q, J=7.2 Hz, 2H), 3.85 (s, 3H), 3.77 (dd, J=4.8, 14.4 Hz, 1H), 3.03-2.92 (m, 2H), 2.84 (s, 3H), 1.67-1.57 (m, 3H), 1.47 (t, J=7.2 Hz, 3H), 1.33-1.23 (m, 12H), 1.14-1.08 (m, 2H), 0.95-0.90 (m, 2H), 0.89-0.86 (m, 3H).

LCMS: 613.1 ([M+H]+).

### EXAMPLE 18

### (S)-2-[1-(3-ethoxy-4-methoxyphenyl)-2-alkylsulfonyl ethyl]-4-propanamido-7-nonyl isoindoline-1, 3-diketone

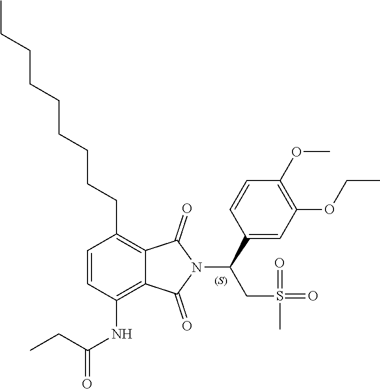

¹H-NMR (400 MHz, CDCl₃) δ=9.56 (s, 1H), 8.66 (d, J=8.8 Hz, 1H), 7.42 (d, J=8.8 Hz, 1H), 7.14-7.07 (m, 2H), 6.84 (d, J=8.8 Hz, 1H), 5.85 (dd, J=4.8, 10.0 Hz, 1H), 4.52 (dd, J=10.0, 14.4 Hz, 1H), 4.11 (q, J=7.2 Hz, 2H), 3.85 (s, 3H), 3.76 (dd, J=4.8, 14.4 Hz, 1H), 2.98 (dd, J=6.8, 8.4 Hz, 2H), 2.84 (s, 3H), 2.48 (q, J=7.6 Hz, 2H), 1.61-1.56 (m, 2H), 1.47 (t, J=7.2 Hz, 3H), 1.34-1.21 (m, 15H), 0.87 (t, J=6.8 Hz, 3H).

LCMS: 601.2 ([M+H]+).

### EXAMPLE 19

### (S)-2-[1-(3-ethoxy-4-methoxyphenyl)-2-alkylsulfonyl ethyl]-4-propanamido-7-tridecyl isoindoline-1, 3-diketone

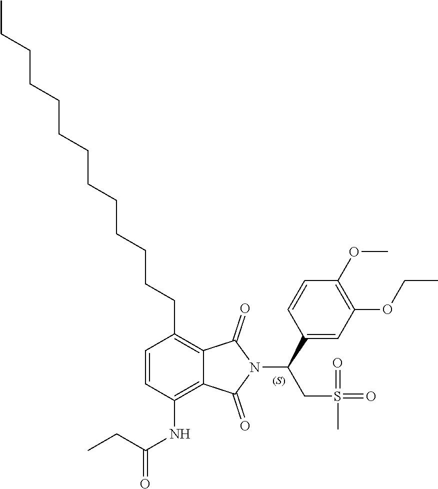

¹H-NMR (400 MHz, CDCl₃) δ=9.56 (s, 1H), 8.66 (d, J=8.8 Hz, 1H), 7.42 (d, J=8.8 Hz, 1H), 7.16-7.06 (m, 2H), 6.84 (d, J=8.8 Hz, 1H), 5.85 (dd, J=4.4, 10.0 Hz, 1H), 4.52 (dd, J=10.0, 14.4 Hz, 1H), 4.11 (q, J=7.2 Hz, 2H), 3.85 (s, 3H), 3.76 (dd, J=4.8, 14.4 Hz, 1H), 2.98 (dd, J=6.8, 8.8 Hz, 2H), 2.84 (s, 3H), 2.48 (q, J=7.6 Hz, 2H), 1.62 (br s, 2H), 1.47 (t, J=7.2 Hz, 2H), 1.49-1.43 (m, 3H), 1.37-1.23 (m, 23H), 0.90-0.84 (m, 3H).

LCMS: 657.3 ([M+H]+).

### EXAMPLE 20

### (S)-2-[1-(3-ethoxy-4-methoxyphenyl)-2-alkylsulfonyl ethyl]-4-(γ-chlorobutyramido)-7-hexyl isoindoline-1, 3-diketone

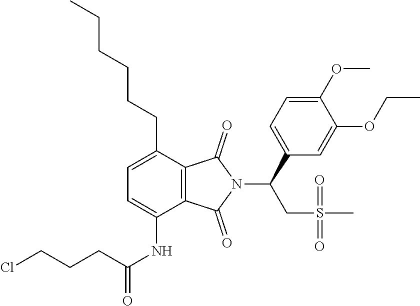 [SMILES: CCCCCCc1ccc(NC(=O)CCCCl)c2c1C(=O)N([C@H](CS(C)(=O)=O)c1ccc(OC)c(OCC)c1)C2=O]

¹H-NMR (400 MHz, CDCl₃) δ=9.58 (s, 1H), 8.63 (d, J=8.4 Hz, 1H), 7.43 (d, J=8.8 Hz, 1H), 7.11 (br d, J=4.4 Hz, 2H), 6.85 (br d, J=8.8 Hz, 1H), 5.86 (br dd, J=4.4, 10.0 Hz, 1H), 4.53 (br dd, J=10.4, 14.4 Hz, 1H), 4.12 (q, J=6.8 Hz, 2H), 3.85 (s, 3H), 3.75 (br dd, J=4.4, 14.4 Hz, 1H), 3.66 (t, J=6.4 Hz, 2H), 2.99 (br t, J=7.6 Hz, 2H), 2.85 (s, 3H), 2.66 (br t, J=7.2 Hz, 2H), 2.22 (quin, J=6.6 Hz, 2H), 1.64-1.57 (m, 2H), 1.47 (t, J=7.2 Hz, 3H), 1.34-1.25 (m, 6H), 0.90-0.84 (m, 3H).

LCMS: 607.1 ([M+H]+).

### EXAMPLE 21

### (S)-2-[1-(3-ethoxy-4-methoxyphenyl)-2-alkylsulfonyl ethyl]-4-(γ-chlorobutyramido)-7-heptyl isoindoline-1, 3-diketone

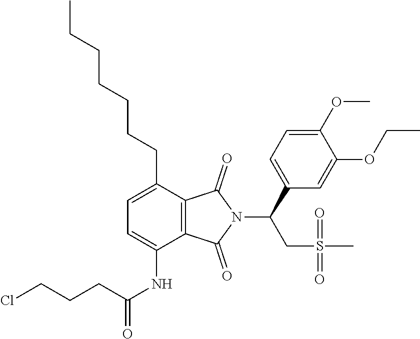 [SMILES: CCCCCCCc1ccc(NC(=O)CCCCl)c2c1C(=O)N([C@H](CS(C)(=O)=O)c1ccc(OC)c(OCC)c1)C2=O]

¹H-NMR (400 MHz, CDCl₃) δ=9.58 (s, 1H), 8.63 (br d, J=8.4 Hz, 1H), 7.42 (br d, J=8.8 Hz, 1H), 7.11 (br s, 2H), 6.85 (br d, J=8.8 Hz, 1H), 5.86 (br dd, J=4.4, 10.0 Hz, 1H), 4.53 (br dd, J=10.4, 14.0 Hz, 1H), 4.12 (q, J=6.8 Hz, 2H), 3.85 (s, 3H), 3.75 (br dd, J=4.4, 14.4 Hz, 1H), 3.66 (br t, J=6.0 Hz, 2H), 3.07-2.91 (m, 2H), 2.85 (s, 3H), 2.65 (br t, J=7.2 Hz, 2H), 2.22 (quin, J=6.5 Hz, 2H), 1.58 (br d, J=6.4 Hz, 2H), 1.47 (br t, J=6.8 Hz, 3H), 1.34-1.23 (m, 8H), 0.87 (br t, J=6.4 Hz, 3H).

LCMS: 621.1 ([M+H]+).

### EXAMPLE 22

### (S)-2-[1-(3-ethoxy-4-methoxyphenyl)-2-alkylsulfonyl ethyl]-4-(γ-chlorobutyramido)-7-octyl isoindoline-1, 3-diketone

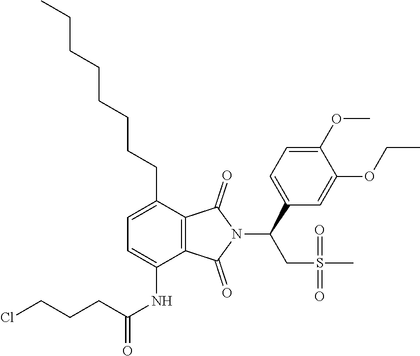 [SMILES: CCCCCCCCc1ccc(NC(=O)CCCCl)c2c1C(=O)N([C@H](CS(C)(=O)=O)c1ccc(OC)c(OCC)c1)C2=O]

¹H-NMR (400 MHz, CDCl₃) δ=9.58 (s, 1H), 8.63 (d, J=8.8 Hz, 1H), 7.42 (d, J=8.8 Hz, 1H), 7.11 (dd, J=2.4, 4.4 Hz, 2H), 6.88-6.82 (m, 1H), 5.86 (dd, J=4.4, 10.0 Hz, 1H), 4.53 (dd, J=10.4, 14.4 Hz, 1H), 4.12 (q, J=7.2 Hz, 2H), 3.85 (s, 3H), 3.75 (dd, J=4.8, 14.4 Hz, 1H), 3.66 (t, J=6.4 Hz, 2H), 3.03-2.91 (m, 2H), 2.85 (s, 3H), 2.66 (t, J=7.2 Hz, 2H), 2.22 (quin, J=6.8 Hz, 2H), 1.64-1.56 (m, 2H), 1.47 (t, J=7.2 Hz, 3H), 1.35-1.23 (m, 10H), 0.87 (t, J=6.8 Hz, 3H).

LCMS: 635.1 ([M+H]+).

### EXAMPLE 23

### (S)-2-[1-(3-ethoxy-4-methoxyphenyl)-2-alkylsulfonyl ethyl]-4-propanamido-6-hexyl isoindoline-1, 3-diketone

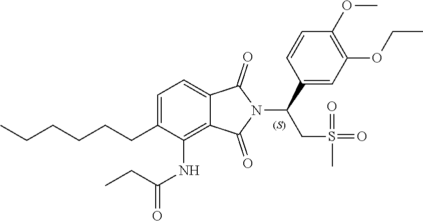

¹H-NMR (400 MHz, CDCl₃) δ=8.00 (br s, 1H), 7.60 (q, J=7.6 Hz, 2H), 7.11-7.05 (m, 2H), 6.83 (d, J=7.6 Hz, 1H), 5.85 (dd, J=4.8, 10.0 Hz, 1H), 4.51 (dd, J=9.6, 14.4 Hz, 1H), 4.10 (q, J=7.2 Hz, 2H), 3.85 (s, 3H), 3.75 (dd, J=4.4, 14.4 Hz, 1H), 2.83 (s, 3H), 2.71-2.63 (m, 2H), 2.51 (q, J=7.2 Hz, 2H), 1.60-1.56 (m, 2H), 1.46 (t, J=7.2 Hz, 3H), 1.32-1.25 (m, 9H), 0.90-0.84 (m, 3H).

LCMS: 559 ([M+H]+).

### EXAMPLE 24

### (S)-2-[1-(3-ethoxy-4-methoxyphenyl)-2-alkylsulfonyl ethyl]-4-(γ-chlorobutyramido)-7-pentyl isoindoline-1, 3-diketone

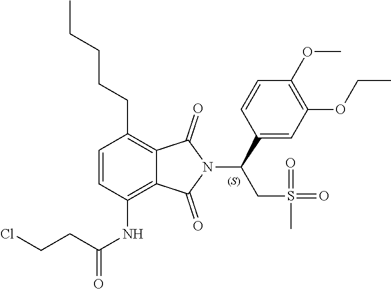

¹H-NMR (400 MHz, CDCl₃) δ=9.63 (s, 1H), 8.65 (d, J=8.8 Hz, 1H), 7.44 (d, J=8.8 Hz, 1H), 7.15-7.07 (m, 2H), 6.85 (d, J=8.8 Hz, 1H), 5.86 (dd, J=4.4, 10.4 Hz, 1H), 4.53 (dd, J=10.4, 14.4 Hz, 1H), 4.12 (q, J=7.2 Hz, 2H), 3.88 (t, J=6.4 Hz, 2H), 3.85 (s, 3H), 3.75 (dd, J=4.4, 14.4 Hz, 1H), 3.03-2.95 (m, 2H), 2.91 (t, J=6.4 Hz, 2H), 2.85 (s, 3H), 1.64-1.56 (m, 2H), 1.47 (t, J=7.2 Hz, 3H), 1.37-1.29 (m, 4H), 0.93-0.85 (m, 3H).

LCMS: 579 ([M+H]+).

### EXAMPLE 25

### (S)-2-[1-(3-ethoxy-4-methoxyphenyl)-2-alkylsulfonyl ethyl]-4-(chloroacetamido)-7-pentyl isoindoline-1, 3-diketone

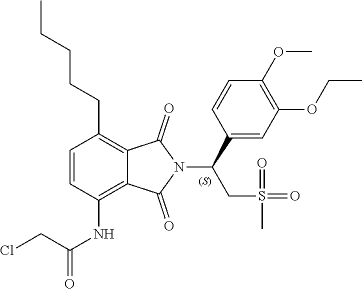

¹H-NMR (400 MHz, CDCl₃) δ=10.57 (s, 1H), 8.64 (d, J=8.4 Hz, 1H), 7.46 (d, J=8.8 Hz, 1H), 7.17-7.09 (m, 2H), 6.85 (d, J=8.0 Hz, 1H), 5.87 (dd, J=4.8, 10.0 Hz, 1H), 4.53 (dd, J=10.4, 14.4 Hz, 1H), 4.21 (s, 2H), 4.16-4.09 (m, 2H), 3.85 (s, 3H), 3.77 (dd, J=4.8, 14.4 Hz, 1H), 3.04-2.98 (m, 2H), 2.84 (s, 3H), 1.63-1.59 (m, 2H), 1.47 (t, J=6.8 Hz, 3H), 1.36-1.31 (m, 4H), 0.91-0.87 (m, 3H).

LCMS: 587 ([M+Na]+).

### EXAMPLE 26

### (S)-2-[1-(3-ethoxy-4-methoxyphenyl)-2-alkylsulfonyl ethyl]-4-chloroacetamido-7-butyl isoindoline-1, 3-diketone

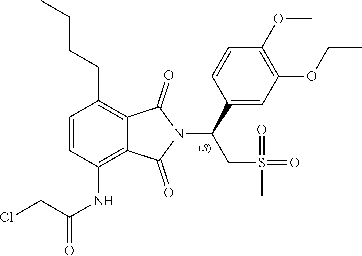

¹H-NMR (400 MHz, CDCl₃) δ=10.57 (s, 1H), 8.64 (d, J=8.4 Hz, 1H), 7.46 (d, J=8.8 Hz, 1H), 7.17-7.10 (m, 2H), 6.85 (d, J=8.4 Hz, 1H), 5.87 (dd, J=4.8, 10.0 Hz, 1H), 4.53 (dd, J=9.6, 14.4 Hz, 1H), 4.21 (s, 2H), 4.17-4.08 (m, 2H), 3.85 (s, 3H), 3.77 (dd, J=4.8, 14.4 Hz, 1H), 3.05-2.98 (m, 2H), 2.85 (s, 3H), 1.65-1.57 (m, 2H), 1.50-1.45 (m, 2H), 1.42-1.35 (m, 2H), 0.94 (t, J=7.2 Hz, 3H).

LCMS: 551 ([M+H]+).

### EXAMPLE 27

### (S)-2-[1-(3-ethoxy-4-methoxyphenyl)-2-alkylsulfonyl ethyl]-4-(γ-chlorobutyramido)-7-butyl isoindoline-1, 3-diketone

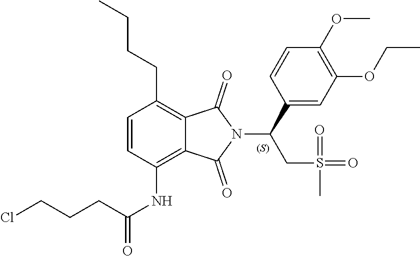

¹H-NMR (400 MHz, CDCl₃) δ=9.59 (s, 1H), 8.63 (d, J=8.8 Hz, 1H), 7.43 (d, J=8.8 Hz, 1H), 7.15-7.07 (m, 2H), 6.85 (d, J=8.8 Hz, 1H), 5.86 (dd, J=4.4, 10.0 Hz, 1H), 4.53 (dd, J=10.0, 14.4 Hz, 1H), 4.12 (q, J=7.2 Hz, 2H), 3.86 (s, 3H), 3.75 (dd, J=4.8, 14.4 Hz, 1H), 3.67 (t, J=6.4 Hz, 2H), 3.04-2.96 (m, 2H), 2.85 (s, 3H), 2.66 (t, J=7.2 Hz, 2H), 2.22 (quin, J=6.8 Hz, 2H), 1.63-1.56 (m, 2H), 1.47 (t, J=7.2 Hz, 3H), 1.43-1.34 (m, 2H), 0.93 (t, J=7.2 Hz, 3H).

LCMS: 579 ([M+H]+).

### EXAMPLE 28

### (S)-2-[1-(3-ethoxy-4-methoxyphenyl)-2-alkylsulfonyl ethyl]-4-fluoroacetamido-7-pentyl isoindoline-1, 3-diketone

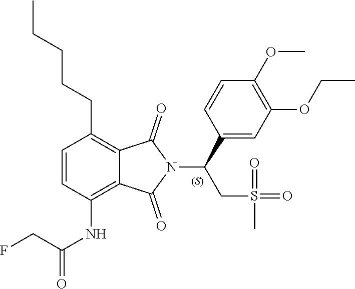

¹H-NMR (400 MHz, CDCl₃) =10.34 (br d, J=4.4 Hz, 1H), 8.65 (d, J=8.4 Hz, 1H), 7.46 (d, J=8.8 Hz, 1H), 7.16-7.10 (m, 2H), 6.84 (d, J=8.8 Hz, 1H), 5.86 (dd, J=4.4, 10.0 Hz, 1H), 5.04-4.86 (m, 2H), 4.54 (dd, J=10.0, 14.4 Hz, 1H), 4.12 (q, J=7.2 Hz, 2H), 3.85 (s, 3H), 3.75 (dd, J=4.4, 14.4 Hz, 1H), 3.01 (dd, J=6.8, 8.8 Hz, 2H), 2.85 (s, 3H), 1.67-1.59 (m, 2H), 1.47 (t, J=7.2 Hz, 3H), 1.39-1.32 (m, 4H), 0.93-0.87 (m, 3H).

LCMS: 571 ([M+Na]+).

### EXAMPLE 29

### (S)-2-[1-(3-ethoxy-4-methoxyphenyl)-2-alkylsulfonyl ethyl]-4-acrylamido-7-pentyl isoindoline-1, 3-diketone

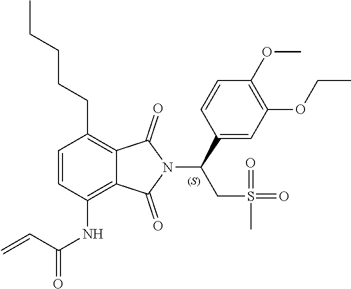

¹H-NMR (400 MHz, CDCl₃) δ=9.73 (s, 1H), 8.73 (d, J=8.8 Hz, 1H), 7.45 (d, J=8.8 Hz, 1H), 7.11 (qd, J=2.0, 4.4 Hz, 2H), 6.85 (d, J=8.8 Hz, 1H), 6.50-6.43 (m, 1H), 6.36-6.27 (m, 1H), 5.90-5.83 (m, 2H), 4.53 (dd, J=10.4, 14.4 Hz, 1H), 4.12 (q, J=6.8 Hz, 2H), 3.85 (s, 3H), 3.76 (dd, J=4.4, 14.4 Hz, 1H), 3.03-2.96 (m, 2H), 2.85 (s, 3H), 1.66-1.57 (m, 2H), 1.47 (t, J=7.2 Hz, 3H), 1.38-1.31 (m, 4H), 0.92-0.85 (m, 3H).

LCMS: 543 ([M+H]+).

### EXAMPLE 30

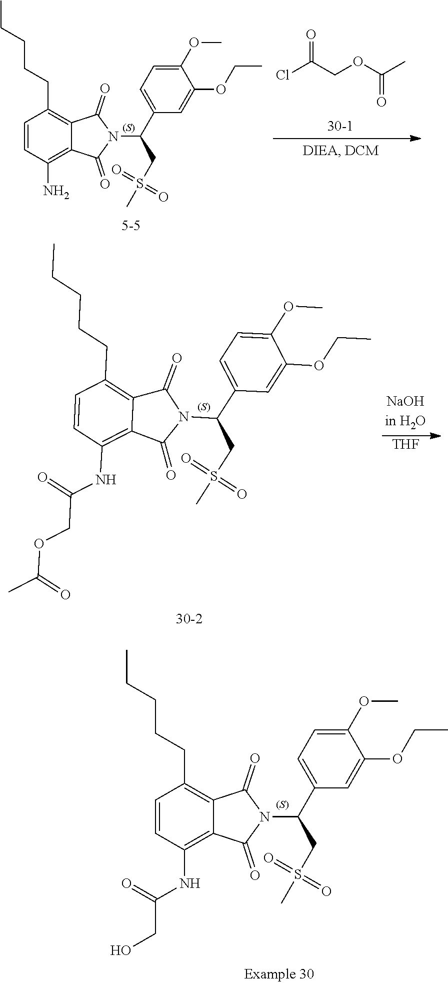

Synthesis of compound 30-2:

The compound 5-5 (200 mg, 0.409 mmol, 1.0 eq) was dissolved in DCM (7 mL), DIEA (158 mg, 1.228 mmol, 3.0 eq) and a compound 30-1 (111.77 mg, 0.818 mmol, 2.0 eq) were added to the reaction solution, and the mixture was stirred at room temperature for 1 h. The reaction solution was quenched with water and then added with water (10 mL), extraction was performed with ethyl acetate (20*2 mL), the combined organic layers were washed with a saturated saline solution, dried with Na₂SO₄, filtered, and concentrated to obtain a crude product, and the crude product was purified by column chromatography (SiO₂, petroleum ether: ethyl acetate=1/0 to 1/1) to obtain a compound 30-2 (150 mg, yield: 62.5%).

¹H-NMR (400 MHz, DMSO) δ 10.10 (s, 1H), 8.42 (d, J=8.6 Hz, 1H), 7.64 (d, J=8.6 Hz, 1H), 7.12 (d, J=1.8 Hz, 1H), 6.97 (dt, J=16.7, 5.1 Hz, 2H), 5.77 (dd, J=10.1, 4.6 Hz, 1H), 4.76 (s, 2H), 4.34 (dd, J=14.3, 10.2 Hz, 1H), 4.16 (dd, J=14.4, 4.7 Hz, 1H), 4.03 (d, J=7.1 Hz, 2H), 3.74 (s, 3H), 3.00 (s, 3H), 2.98-2.89 (m, 2H), 2.24 (s, 3H), 1.66-1.47 (m, 2H), 1.33 (d, J=6.9 Hz, 3H), 1.30-1.22 (m, 4H), 0.85 (t, J=6.8 Hz, 3H).

Synthesis of Example 30:

The compound 30-2 (150 mg, 0.255 mmol, 1.0 eq) was dissolved in THF (5 mL) and H₂O (2.5 mL), NaOH (2.5 g) was added, and the mixture was stirred at room temperature for 1 h. The reaction solution was added with water (20 mL), and extracted with ethyl acetate (20 mL*2); the combined organic layers were washed with a saturated saline solution, dried with Na₂SO, filtered, and concentrated to obtain a crude product; and the crude product was purified by using prep-TLC (SiO₂, dichloromethane:methanol=10:1) to obtain Example 30 (43.86 mg, yield: 31.49%).

¹H NMR (400 MHz, DMSO) δ 10.66 (s, 1H), 8.65 (d, J=8.6 Hz, 1H), 7.64 (d, J=8.7 Hz, 1H), 7.08 (d, J=1.9 Hz, 1H), 6.98 (dt, J=19.0, 5.2 Hz, 2H), 6.32 (t, J=5.6 Hz, 1H), 5.77 (dd, J=10.3, 4.4 Hz, 1H), 4.35 (dd, J=14.3, 10.5 Hz, 1H), 4.15 (dd, J=14.3, 4.5 Hz, 1H), 4.09-3.98 (m, 4H), 3.74 (s, 3H), 3.02 (d, J=5.9 Hz, 3H), 2.99-2.88 (m, 2H), 1.66-1.41 (m, 2H), 1.35-1.23 (m, 7H), 0.85 (t, J=6.9 Hz, 3H).

LCMS: (M+H)+: 547.

### EXAMPLE 31

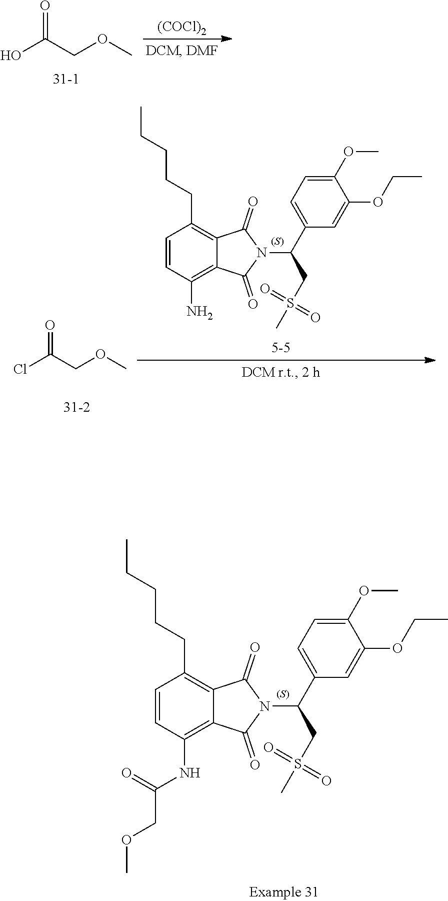

Synthesis of compound 31-2:

A compound 31-1 (1.0 g, 11.1 mmol, 1.0 eq) was dissolved in DCM (15 mL), (COC1)₂ (1.8 g, 14.4 mmol, 1.3 eq) was added at 0° C., followed by a catalytic amount of DMF (0.1 mL), and the mixture was stirred at room temperature for 18 h. The reaction solution was directly used in the next step.

Synthesis of Example 31:

The compound 5-5 (100 mg, 0.20 mmol, 1.0 eq) was dissolved in DCM (5 mL), DIEA (400 mg, 3.05 mmol, 5.0 eq) and the compound 31-2 (1.0 mL) were added, and the mixture was stirred at room temperature for 2 h. The reaction solution was added with water (10 mL), and extracted with ethyl acetate (20 mL*2); the combined organic layers were washed with a saturated saline solution, dried with Na₂SO₄, filtered, and concentrated to obtain a crude product; and the crude product was purified by using prep-HPLC (a formic acid system) to obtain Example 31 (15.28 mg, yield: 10%).

¹H-NMR (400 MHz, CDCl₃) δ 10.47 (s, 1 H), 8.70 (d, J=8.4 Hz, 1H), 7.45 (d, J=8.4 Hz, 1 H), 7.14-7.12 (m, 2H), 6.85 (d, J=8.4 Hz, 1H),5.88 (q, J=4.8 Hz, 1 H),4.52-4.47 (m, 1 H), 4.13-4.12(m, 2 H),4.06 (d, J=1.0 Hz, 2H), 3.85 (s, 3 H), 3.81 (dd, J=5.2 Hz, 14.4 Hz, 1H),3.58 (s, 3H), 2.99 (t, J -8.0 Hz, 2H),2.82 (s, 3 H), 1.48 (t, J=10.2 Hz, 4H), 1.35-1.32 (m, 5H), 1.26(s, 1H), 0.92-0.87 (m, 3H).

LCMS: (M+H)+:561.

### EXAMPLE 32

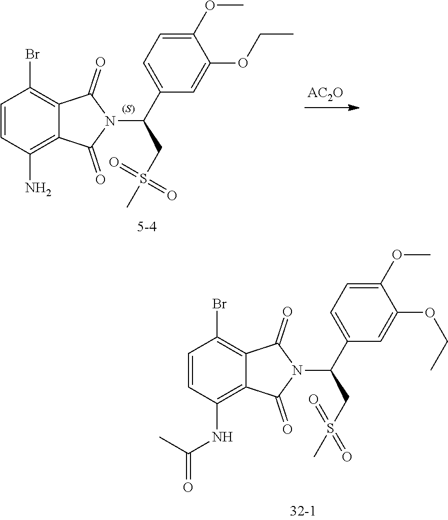

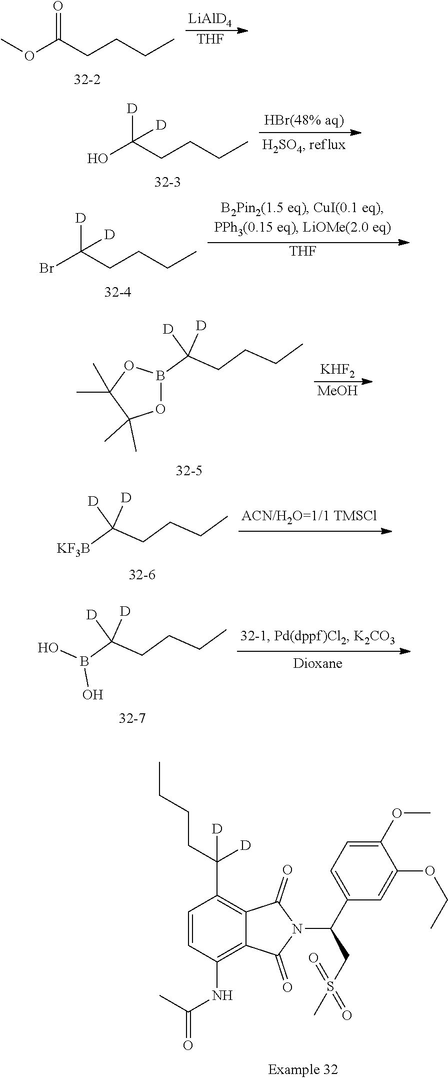

Synthesis of compound 32-1:

The compound 5-4 (2 g, 4.12 mmol, 1.0 eq) was dissolved in acetic anhydride (9 mL) and stirred at 110° C. for 2 h. The reaction solution was concentrated to obtain a residue, the residue was slurried with methyl tert-butyl ether/ethyl acetate (20 mL/10 mL) and then filtered, and the filter cake was washed with methyl tert-butyl ether to obtain a compound 32-1 (1.8 g, 3.34 mmol, yield: 80.99%).

Synthesis of compound 32-3:

A compound 32-2 (8.05 g, 69.30 mmol, 1.0 eq) was dissolved in THF (100 mL), LiAlD₄ (3.2 g, 41.98 mmol, 1.1 eq) was added at 0° C., and the mixture was gradually heated up to room temperature and stirred for 5 h. The reaction solution was quenched with ethyl acetate (4 mL) and then concentrated. The solid was suspended in ethyl acetate (100 mL) at 0° C., a small amount of cold water (80 mL) was added into the suspension, the pH of the solution was adjusted to 1 with 2M HCl, and extraction was carried out with ethyl acetate (60 mL*2). the combined organic layers were washed with a saturated saline solution, dried with Na₂SO₄, filtered, and concentrated to obtain a compound 32-3 (5.13 g, 56.89 mmol, yield: 82.11%).

Synthesis of compound 32-4:

HBr (14.62 g, 40% purity, 72.26 mmol, 1.27 eq) was dissolved in H2SO₄ (3.64 mL), the compound 32-3 (5.13 g, 56.89 mmol, 1 eq) was added, and the mixture was stirred at 120° C. for 2 h. The reaction solution was added with water (500 mL), and extracted with ethyl acetate (100 mL*2); the combined organic layers were washed with a saturated saline solution, dried with Na₂SO₄, filtered, and concentrated to obtain a compound 32-4 (1.79 g, 11.69 mmol, yield: 20.56%).

Synthesis of compound 32-5:

B2Pin2 (3.199 g, 12.64 mmol, 1.5 eq), CuI (160 mg, 0.84 mmol, 0.1 eq), LiO^{t}Bu (1.349 g, 16.86 mmol, 2 eq) were dissolved in tetrahydrofuran (10 mL), the compound 32-4 (1.29 g, 8.42 mmol, 1 eq) was added, and the mixture was stirred at room temperature under nitrogen atmosphere for 16 h. The reaction solution was filtered and concentrated to obtain a crude product, and the crude product was purified by column chromatography (silica, hexane:ethyl acetate=100:1 to 50:1) to obtain a compound 32-5 (1.28 g, 6.39 mmol, yield: 75.96%).

Synthesis of compound 32-6:

The compound 32-5 (400 mg, 1.99 mmol, 1.0 eq) was dissolved in methanol (5 mL), KHF₂ (4.5 mL, 19.99 mmol, 4.5M, 10 eq) was added, and the mixture was stirred at room temperature for 16 h. The reaction solution was concentrated to obtain a crude product, which was dissolved and filtered with hot acetone (10 mL); the combined filtrate was concentrated to 4 mL, and then added with ether (10 mL); and the precipitated white solid was filtered, and the filter cake was dried to obtain a compound 32-6 (260 mg, 1.44 mmol, yield: 72.36%).

Synthesis of compound 32-7:

The compound 32-6 (260 mg, 1.44 mmol, 1.0 eq) was dissolved in acetonitrile/water (2 mL/1 mL), trimethoxy chlorosilane (468 mg, 4.31 mmol, 3 eq) was added, and the mixture was stirred at room temperature for 16 h. The reaction solution was diluted with a saturated sodium bicarbonate solution (10 mL), and extracted with ethyl acetate (10 mL*2); and the combined organic layers were washed with a saturated saline solution, dried with Na₂SO₄, filtered, and concentrated to obtain a compound 32-7 (30 mg, 0.25 mmol, yield: 17.66%).

Synthesis of Example 32

The compound 32-7 (30 mg, 0.25 mmol, 2 eq) was dissolved in dioxane (0.5 mL), the compound 32-1 (68.5 mg, 0.127 mmol, 1 eq), K₂CO₃ (52.6 mg, 0.381 mmol, 3 eq) and Pd(dppf)Cl₂ (4.6 mg, 6.35 μmol, 0.05 eq) were added, and the mixture was stirred at 100° C. for 4 h under nitrogen atmosphere. The reaction solution was added with water (10 mL), and extracted with ethyl acetate (3 mL*2); the combined organic layers were washed with a saturated saline solution, dried with Na₂SO₄, filtered, and concentrated to obtain a crude product; and the crude product was purified by using prep-HPLC (a formic acid system) to obtain Example 32 (24.87 mg, 0.047 mmol, yield: 36.81%).

¹H NMR (400 MHz, CDCl₃) δ 9.53 (s, 1H), 8.64 (d, J=8 Hz, 1H), 7.42 (d, J=8 Hz, 1H), 7.10 (s, 2H), 6.85 (d, J=8 Hz, 1H), 5.86 (m, 1H), 4.53 (m, 1H), 4.11 (q, 2H), 3.85 (s, 3H), 3.74 (m, 1H), 2.85 (s, 3H), 2.25 (s, 3H), 1.47 (t, 3H), 1.36-1.31 (m, 4H), 1.26 (d, J=4 Hz, 2H), 0.88 (t, 3H).

LCMS: (M+H)+:533.3

### EXAMPLE 33

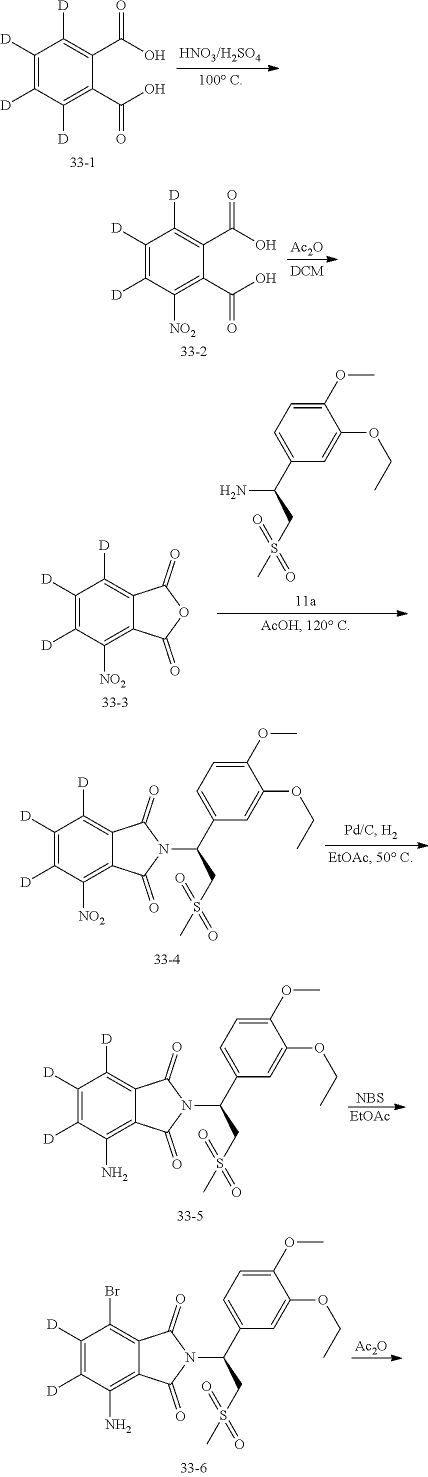

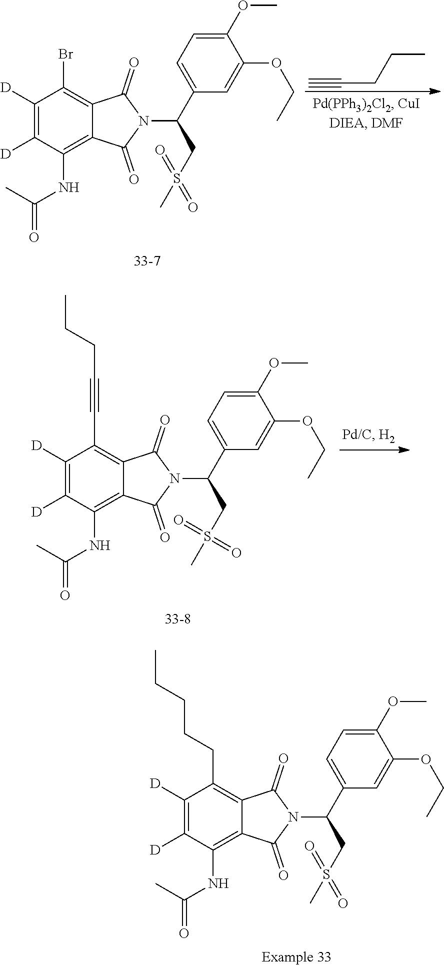

Synthesis of compound 33-2: A compound 33-1 (1 g, 5.88 mmol, 1 eq) was dissolved in H2SO₄ (3.75 mL), and HNO3 (1.48 g, 23.51 mmol, 1.06 mL, 4 eq) was added. The reaction solution was stirred at 100° C. for 3 h. The reaction solution was slowly poured into ice water (50 mL), and extracted with ethyl acetate (50 mL*3); the combined organic layers were washed with a saturated saline solution, dried with Na₂SO₄, filtered, and concentrated to obtain a crude product; and the crude product was purified by column chromatography to obtain a compound 33-2 (970 mg, 4.08 mmol, yield: 69.37%).

LCMS (ESI+): m/z 258.9 (M+1+46)+

Synthesis of compound 33-3: The compound 33-2 (970 mg, 4.53 mmol, 1.0 eq) was dissolved in acetic anhydride (15 mL), and stirred at 120° C. for 2 h. The reaction solution was concentrated to obtain a crude product, which was a compound 33-3 (888 mg, 4.53 mmol, yield: 99.96%) directly used in the next step.

Synthesis of compound 33-4: The compound 11a (1.36 g, 4.98 mmol, 1.1 eq) and the compound 33-3 (888 mg, 4.53 mmol, 1 eq) were dissolved in AcOH (15 mL), and stirred at 120° C. for 16 h. The reaction solution was concentrated to obtain a crude product, and the crude product was purified by column chromatography (petroleum ether:ethyl acetate 10/1 to 4:1) to obtain a compound 33-4 (680 mg, 1.51 mmol, yield: 33.27%).

LCMS (ESI+): m/z 451.5 (M+1)+:

Synthesis of compound 33-5: The compound 33-4 (580 mg, 1.28 mmol, 1 eq) was dissolved in ethyl acetate (5 mL), Pd/C (100 mg, 7.71 mmol, 10% purity) was added under hydrogen atmosphere, and the mixture was stirred at 50° C. under hydrogen (15 psi) atmosphere for 3 h. The reaction solution was filtered and concentrated to obtain a compound 33-5 (520 mg, 1.23 mmol, yield: 96.03%).

LCMS (ESI+): m/z 451.5 (M+1)+

Synthesis of compound 33-6: The compound 33-5 (520 mg, 1.23 mmol, 1 eq) was dissolved in ethyl acetate (10 mL), NBS (219.59 mg, 1.23 mmol, 1 eq) was added, and the mixture was stirred at 25° C. for 4 h. Water (30 mL) and ethyl acetate (3*30 mL) were added for extraction, the combined organic layers were washed with a saturated saline solution, dried with Na₂SO₄, filtered, and concentrated to obtain a crude product, and the crude product was purified by column chromatography (SiO₂, petroleum ether:ethyl acetate=100/0 to 1/1) to obtain a compound 33-6 (534 mg, 1.07 mmol, yield: 86.67%).

LCMS (ESI+): m/z 499.6 (M+1)+

Synthesis of compound 33-7: The compound 33-6 (534 mg, 1.07 mmol, 1 eq) was dissolved in acetic anhydride (190.33 mg, 1.86 mmol, 174.61 μL, 1.74 eq), the reaction solution was stirred at 120° C. for 3 h, and then the reaction solution was concentrated to obtain a crude compound 33-7 (450 mg, 831.17 μmol, yield: 77.73%).

Synthesis of compound 33-8: The compound 33-7 (100 mg, 184.70 μmol, 1 eq), CuI (7.04 mg, 36.94 μmol, 0.2 eq), Pd(PPh3)2Cl₂ (25.93 mg, 36.94 μmol, 0.2 eq), DIEA (71.61 mg, 554.11 μmol, 96.52 μL, 3 eq) and 1-pentyne (125.81 mg, 1.85 mmol, 181.29 μL, 10 eq) were dissolved in DMF (4 mL); the reaction solution was stirred at 60° C. under N₂ atmosphere for 16 h; water (5 mL) and ethyl acetate (5 mL*3) were added into the reaction system for extraction; the combined organic layers were washed with a saturated saline solution, dried with Na₂SO₄, filtered, and concentrated to obtain a crude product; and the crude product was purified by column chromatography (SiO₂, petroleum ether:ethyl acetate 1/0 to 1:1) to obtain a compound 33-8 (75 mg, 141.88 μmol, yield: 76.82%).

LCMS (ESI+): m/z 529.4 (M+1)+

Synthesis of Example 33: The compound 33-8 (75 mg, 141.88 μmol, 1 eq) was dissolved in EtOAc (5 mL), Pd/C (160 mg, 141.88 μmol, 10% purity) was added under nitrogen atmosphere, and the mixture was replaced with hydrogen for 3 times under vacuum condition, and stirred at 50° C. under hydrogen atmosphere (50 Psi) for 16 h. The reaction solution was filtered with diatomite to remove solids, the filter cake was washed with EtOAc, the filtrate was concentrated to obtain a crude product, and the crude product was purified by using prep-HPLC (a formic acid system) to obtain Example 33 (19 mg, 35.67 μmol, yield: 25.14%).

1H NMR (400 MHz, DMSO-d6) δ 0.85 (s, 3H), 1.22-1.36 (m, 7H), 1.44-1.66 (m, 2H), 2.17 (s, 3H), 2.95 (t, J=7.15 Hz, 2H), 3.01 (s, 3H) 3.73 (s, 3H), 4.01 (d, J=6.85 Hz, 2H), 4.08-4.19 (m, 1H), 4.28-4.41 (m, 1H), 5.76 (d, J=5.99 Hz, 1H), 6.89-7.01 (m, 2H), 7.07 (s, 1H) 9.68 (s, 1H).

LCMS (ESI+): m/z 533.1 (M+1)+

### EXAMPLE 55

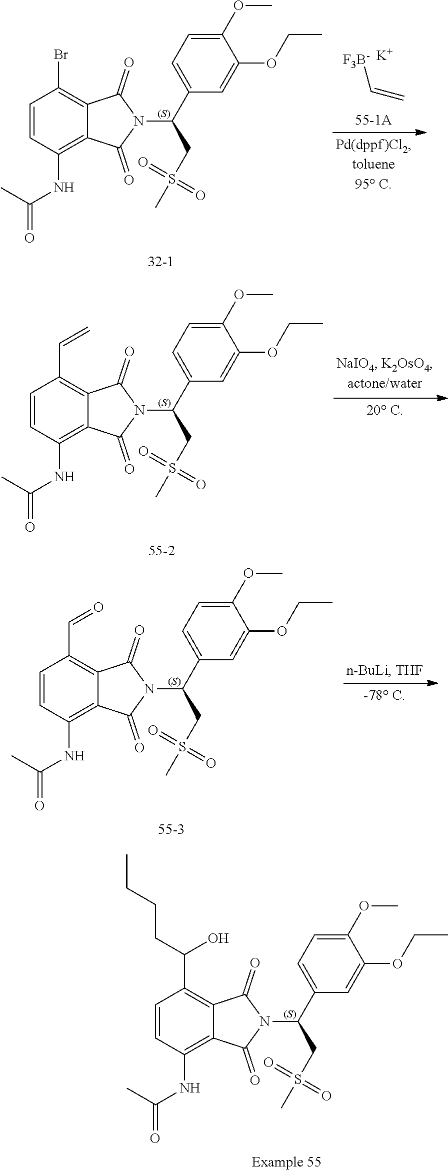

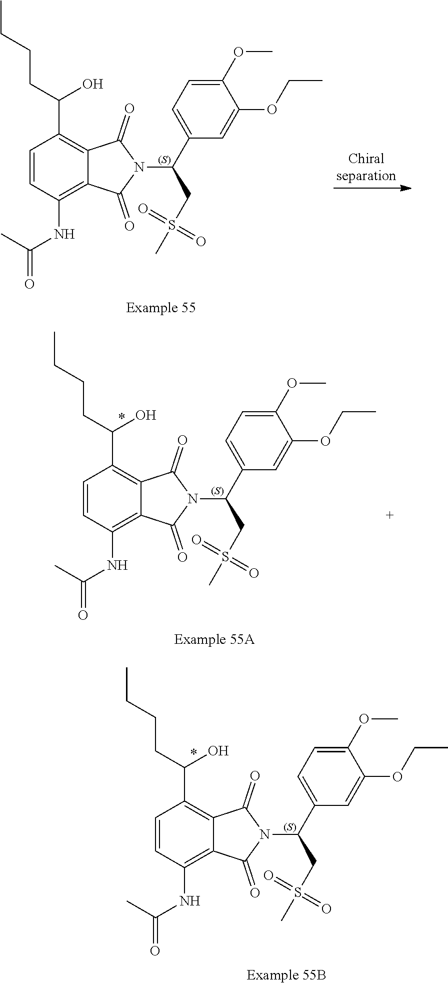

Synthesis of compound 55-2: The compound 32-1 (5 g, 9.27 mmol) was dissolved in dioxane (60 mL) and water (15 mL); Pd(dppf)Cl₂ (900 mg, 1.23 mmol), a compound 55-1A, and potassium phosphate (6.89 g, 32.4 mmol) were added into the reaction solution under nitrogen atmosphere, and the mixture was replaced with nitrogen and kept under nitrogen atmosphere; and the product was stirred at 95° C. for 16 h, diluted with water (100 mL), extracted with ethyl acetate (200 mL*2), and then washed with a saturated saline solution (200 mL). The organic phases were combined, and the combined organic layers were dried with anhydrous Na₂SO₄, filtered, concentrated under reduced pressure, and purified by column chromatography, so that a yellow solid compound 55-2 (4.2 g, 7.77 mmol, yield: 83.8%) was obtained.

LCMS (ESI+): m/z 478.0 (M+1)+:

Synthesis of compound 55-3: The compound 55-2 (4.2 g, 8.63 mmol, 1 eq) was dissolved in acetone (400 mL), dichloromethane (150 mL) and water (150 mL), K₂O_{S}O₄ (1.26 g, 3.42 mmol, 0.4 eq) was added to the reaction solution, the mixture was stirred at 0° C. for 5 min, and NaIO₄ (7.39 g, 34.6 mmol, 1.92 mL, 4 eq) was added to the reaction solution, and stirred at 20° C. for 6 h. The mixture was diluted with water (100 mL), extracted with ethyl acetate (1000 mL), and washed with a saturated saline solution (300 mL). The organic phases were combined, and the combined organic layers were dried with anhydrous Na₂SO₄, filtered, concentrated under reduced pressure, and purified by column chromatography, so that a yellow solid compound 55-3 (3.5 g, 6.45 mmol, yield: 74.7%) was obtained.

LCMS (ESI+): m/z 489.3 (M+1)+: 1.34 min.

Synthesis of Example 55: The compound 55-3 (3.5 g, 7.16 mmol, 1 eq) was dissolved in THF (10 mL), the reaction solution was cooled to −78° C., and n-BuLi (1.6M, 13.5 mL, 3 eq) was added dropwise into the reaction solution; the reaction solution was stirred at −78° C. for 1 h, the reaction solution was quenched with a saturated NH₄Cl solution (20 mL), and then diluted with ethyl acetate (200 mL); and the organic phases were combined, and the obtained combined organic layers were washed with a saturated saline solution (50 mL), dried with anhydrous Na₂SO₄, filtered, and purified by using pre-HPLC (FA), so that a pair of diastereoisomeric compounds in Example 55 (289.80.74 mg, 0.53 mmol, yield: 7.4%) were obtained. Example 55 was subjected to chiral separation to obtain Example 55A (107.1 mg, 99% purity) and Example 55B (117.5 mg, 99% purity).

1H NMR (400 MHz, CDCl₃) of Example 55: δ ppm 0.79-0.84 (m, 3H), 1.19-1.32 (m, 4 H), 1.41 (s, 3H), 1.69-1.80 (m, 1H), 1.70-1.76 (m, 1H), 2.19 (s, 3H), 2.80 (s, 3H), 3.67 (dt, J=14.4, 5.1 Hz, 1H), 3.79 (s, 3H), 4.04 (q, J=7.0 Hz, 2H), 4.41-4.50 (m, 1H), 4.96 (br d, J=5.4 Hz, 1H), 5.77-5.84 (m, 1H), 6.76-6.81 (m, 1H), 6.99-7.05 (m, 2H), 7.19 (s, 7H), 7.54 (d, J=8.8 Hz, 1H), 8.64 (d, J=8.3 Hz, 1H), 9.48 (s, 1H).

LCMS (ESI+): m/z 569.3 (M+Na)+

Synthesis of Example 56:

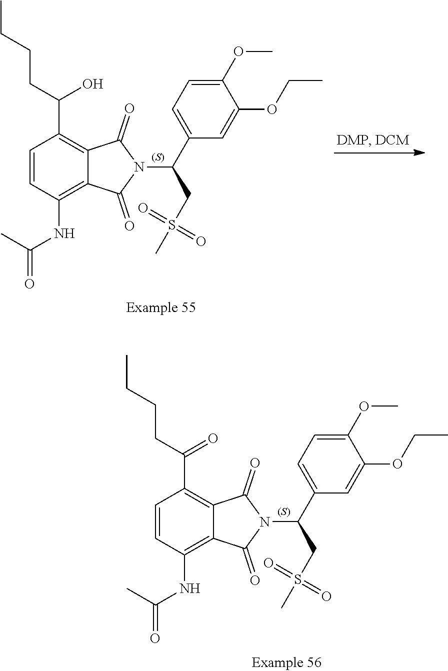

Synthesis of Example 56: The compound 55 (60 mg, 0.11 mmol, 1 eq) was dissolved in DCM (1 mL), DMP (120 mg, 0.28 mmol, 2.6 eq) was added, and the mixture was stirred at room temperature for 1 h; the reaction solution was diluted with DCM (30 mL), a saturated NaHCO₃ solution (3 mL) and a saturated NH₄Cl (3 mL); and the organic phases were combined, and the obtained combined organic layers were washed with a saturated saline solution (30 mL), dried with anhydrous Na₂SO₄, filtered, and purified by using pre-HPLC (FA), so that a compound in Example 56 (43.1 mg, 0.08 mmol, yield: 72.1%) was obtained.

1H NMR (400 MHz, CDCl₃) δ ppm 0.86 (t, J=7.3 Hz, 3H), 1.27-1.36 (m, 2H), 1.41 (s, 3H), 1.57-1.66 (m, 2H), 2.15-2.30 (m, 3H), 2.78-2.88 (m, 3H), 2.98 (t, J=7.4 Hz, 2H), 3.63 (dd, J=14.3, 4.3 Hz, 1H), 3.79 (s, 3H), 4.04 (q, J=7.0 Hz, 2H), 4.48 (dd, J=14.3, 10.8 Hz, 1 H), 5.81 (dd, J=10.7, 4.2 Hz, 1H), 6.78 (d, J=8.2 Hz, 1H), 6.96-7.07 (m, 2H), 7.61 (d, J=8.7 Hz, 1H), 8.73 (d, J=8.7 Hz, 1H), 9.66 (s, 1H).

LCMS (ESI+): m/z 545.3 (M+H)+

### EXAMPLE 34-54

| | | | Reference |
|---|---|---|---|
| Compound | | | example for |
| No. | Compound structure | (M + H)⁺ | synthesis method |
| Example 34 | 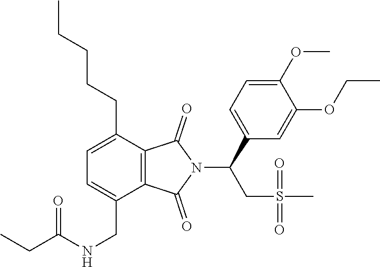 [SMILES: CCCCCc1ccc(CNC(=O)CC)c2c1C(=O)N([C@H](CS(C)(=O)=O)c1ccc(OC)c(OCC)c1)C2=O] | 559.1 | 4 |
| Example 35 | 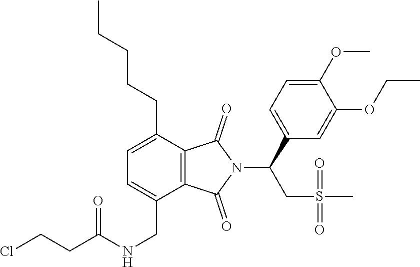 [SMILES: CCCCCc1ccc(CNC(=O)CCCl)c2c1C(=O)N([C@H](CS(C)(=O)=O)c1ccc(OC)c(OCC)c1)C2=O] | 593.1 | 4 |
| Example 36 | 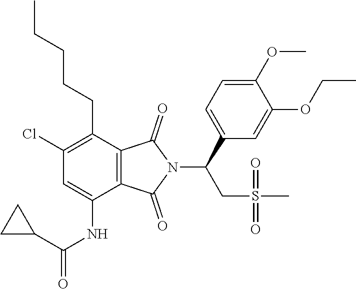 [SMILES: CCCCCc1c(Cl)cc(NC(=O)C2CC2)c2c1C(=O)N([C@H](CS(C)(=O)=O)c1ccc(OC)c(OCC)c1)C2=O] | 591 | 5 |
| Example 37 | 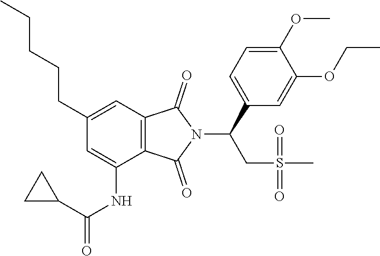 [SMILES: CCCCCc1cc(NC(=O)C2CC2)c2c(c1)C(=O)N([C@H](CS(C)(=O)=O)c1ccc(OC)c(OCC)c1)C2=O] | 557.1 | 2 |
| Example 38 | 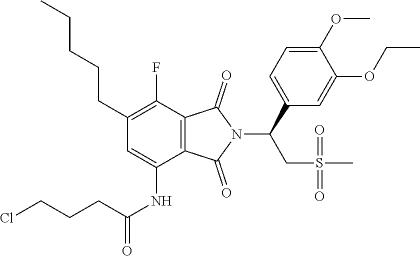 [SMILES: CCCCCc1cc(NC(=O)CCCCl)c2c(c1F)C(=O)N([C@H](CS(C)(=O)=O)c1ccc(OC)c(OCC)c1)C2=O] | 611.1 | 2 |
| Example 39 | 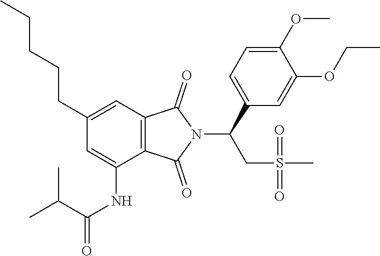 [SMILES: CCCCCc1cc(NC(=O)C(C)C)c2c(c1)C(=O)N([C@H](CS(C)(=O)=O)c1ccc(OC)c(OCC)c1)C2=O] | 559.1 | 2 |
| Example 40 | 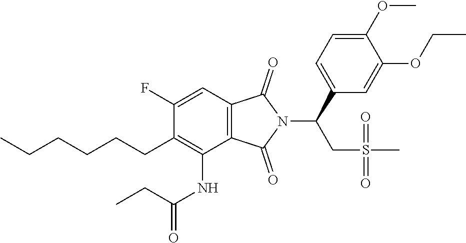 [SMILES: CCCCCCc1c(F)cc2c(c1NC(=O)CC)C(=O)N([C@H](CS(C)(=O)=O)c1ccc(OC)c(OCC)c1)C2=O] | 577.1 | 1 |
| Example 41 | 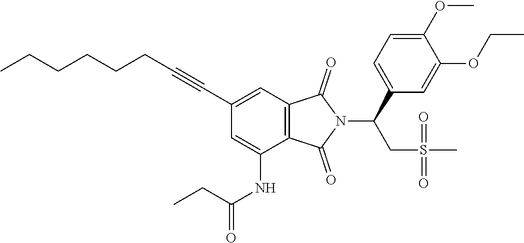 [SMILES: CCCCCCC#Cc1cc(NC(=O)CC)c2c(c1)C(=O)N([C@H](CS(C)(=O)=O)c1ccc(OC)c(OCC)c1)C2=O] | 583.1 | 3 |
| Example 42 | 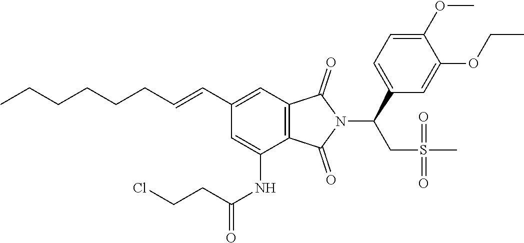 [SMILES: CCCCCC/C=C/c1cc(NC(=O)CCCl)c2c(c1)C(=O)N([C@H](CS(C)(=O)=O)c1ccc(OC)c(OCC)c1)C2=O] | 619.1 | 3 |
| Example 43 | 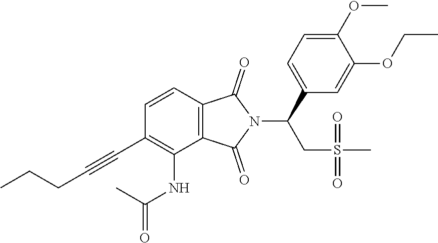 [SMILES: CCCC#Cc1ccc2c(c1NC(C)=O)C(=O)N([C@H](CS(C)(=O)=O)c1ccc(OC)c(OCC)c1)C2=O] | 527.1 | 1 |
| Example 44 | 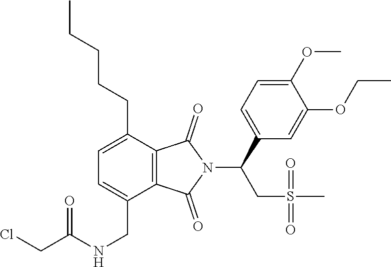 [SMILES: CCCCCc1ccc(CNC(=O)CCl)c2c1C(=O)N([C@H](CS(C)(=O)=O)c1ccc(OC)c(OCC)c1)C2=O] | 579.1 | 4 |
| Example 45 | 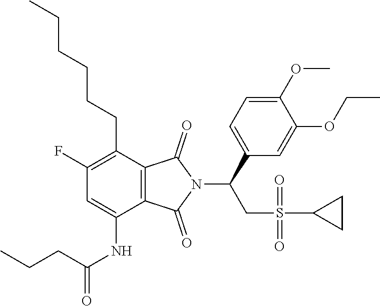 [SMILES: CCCCCCc1c(F)cc(NC(=O)CCC)c2c1C(=O)N([C@H](CS(=O)(=O)C1CC1)c1ccc(OC)c(OCC)c1)C2=O] | 617.1 | 5 |
| Example 46 | 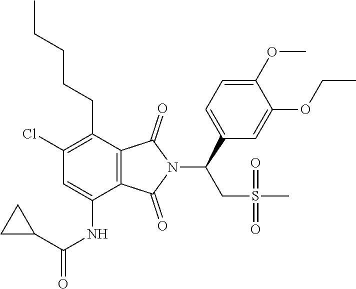 [SMILES: CCCCCc1c(Cl)cc(NC(=O)C2CC2)c2c1C(=O)N([C@H](CS(C)(=O)=O)c1ccc(OC)c(OCC)c1)C2=O] | 591.1 | 5 |
| Example 47 | 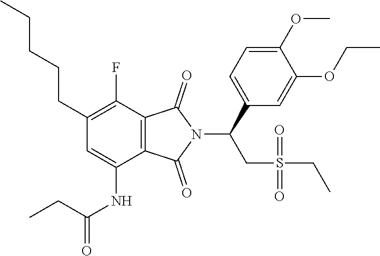 [SMILES: CCCCCc1cc(NC(=O)CC)c2c(c1F)C(=O)N([C@H](CS(=O)(=O)CC)c1ccc(OC)c(OCC)c1)C2=O] | 577.2 | 3 |
| Example 48 | 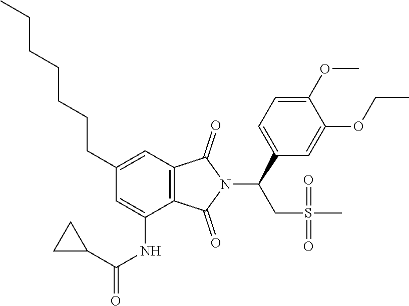 [SMILES: CCCCCCCc1cc(NC(=O)C2CC2)c2c(c1)C(=O)N([C@H](CS(C)(=O)=O)c1ccc(OC)c(OCC)c1)C2=O] | 584.1 | 3 |
| Example 49 | 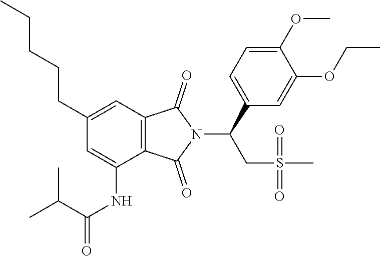 [SMILES: CCCCCc1cc(NC(=O)C(C)C)c2c(c1)C(=O)N([C@H](CS(C)(=O)=O)c1ccc(OC)c(OCC)c1)C2=O] | 559.2 | 3 |
| Example 50 | 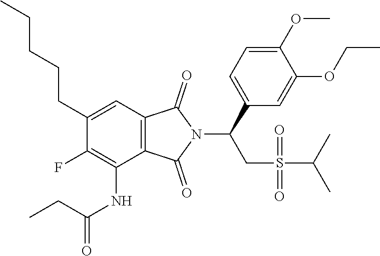 [SMILES: CCCCCc1cc2c(c(NC(=O)CC)c1F)C(=O)N([C@H](CS(=O)(=O)C(C)C)c1ccc(OC)c(OCC)c1)C2=O] | 591.1 | 3 |
| Example 51 | 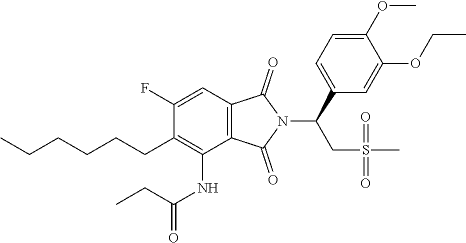 [SMILES: CCCCCCc1c(F)cc2c(c1NC(=O)CC)C(=O)N([C@H](CS(C)(=O)=O)c1ccc(OC)c(OCC)c1)C2=O] | 577.1 | 1 |
| Example 52 | 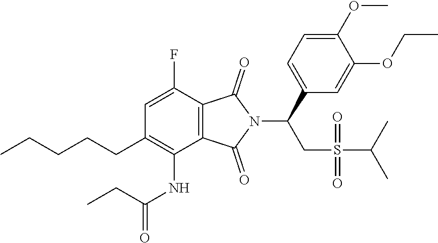 [SMILES: CCCCCc1cc(F)c2c(c1NC(=O)CC)C(=O)N([C@H](CS(=O)(=O)C(C)C)c1ccc(OC)c(OCC)c1)C2=O] | 591.1 | 1 |
| Example 53 | 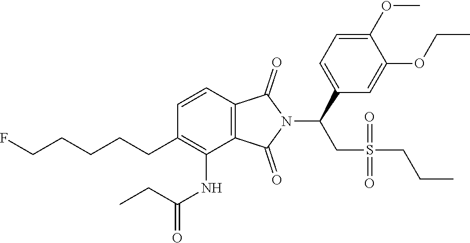 [SMILES: CCCS(=O)(=O)C[C@H](c1ccc(OC)c(OCC)c1)N1C(=O)c2ccc(CCCCCF)c(NC(=O)CC)c2C1=O] | 591.1 | 1 |
| Example 54 | 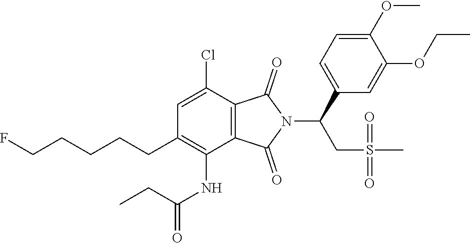 [SMILES: CCOc1cc([C@@H](CS(C)(=O)=O)N2C(=O)c3c(Cl)cc(CCCCCF)c(NC(=O)CC)c3C2=O)ccc1OC] | 597.1 | 1 |

Refer to Examples 9, 55, or 56 for synthesizing the following compounds in Examples 57-82

| Compound No. | Compound structure | (M + H)⁺ |
|---|---|---|
| Example 57 | 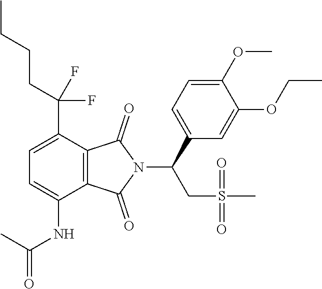 [SMILES: CCCCC(F)(F)c1ccc(NC(C)=O)c2c1C(=O)N([C@H](CS(C)(=O)=O)c1ccc(OC)c(OCC)c1)C2=O] | 567.1 |
| Example 58 | 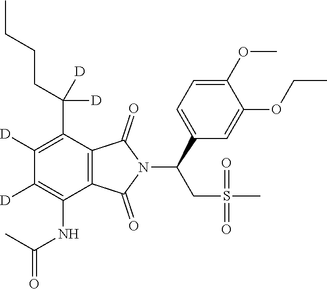 [SMILES: [2H]c1c([2H])c(C([2H])([2H])CCCC)c2c(c1NC(C)=O)C(=O)N([C@H](CS(C)(=O)=O)c1ccc(OC)c(OCC)c1)C2=O] | 535.2 |
| Example 59 | 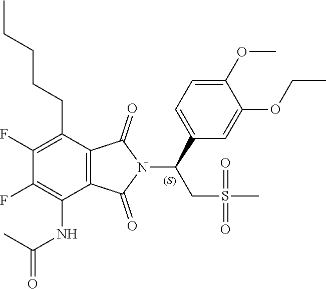 | 567.1 |
| Example 60 | 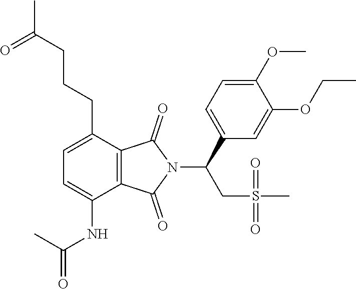 [SMILES: CCOc1cc([C@@H](CS(C)(=O)=O)N2C(=O)c3c(CCCC(C)=O)ccc(NC(C)=O)c3C2=O)ccc1OC] | 545.3 |
| Example 61 | 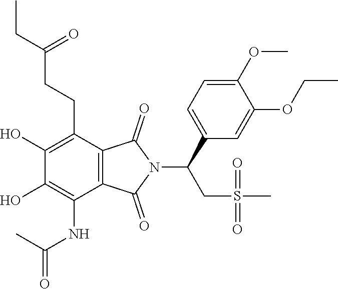 [SMILES: CCOc1cc([C@@H](CS(C)(=O)=O)N2C(=O)c3c(CCC(=O)CC)c(O)c(O)c(NC(C)=O)c3C2=O)ccc1OC] | 599.1 (M + Na)+ |
| Example 62 | 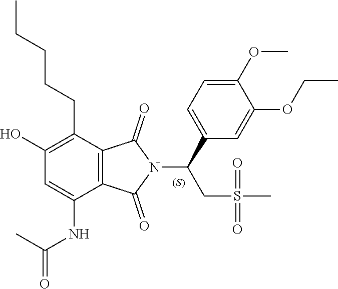 | 569.3 (M + Na)+ |
| Example 63 | 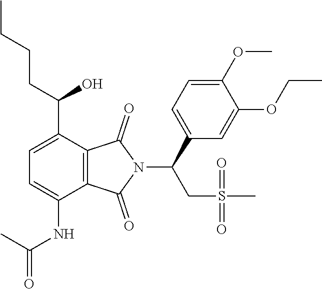 [SMILES: CCCC[C@@H](O)c1ccc(NC(C)=O)c2c1C(=O)N([C@H](CS(C)(=O)=O)c1ccc(OC)c(OCC)c1)C2=O] | 569.3 (M + Na)+ |
| Example 64 | 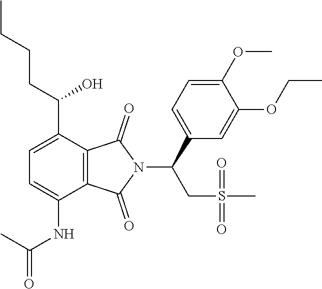 [SMILES: CCCC[C@H](O)c1ccc(NC(C)=O)c2c1C(=O)N([C@H](CS(C)(=O)=O)c1ccc(OC)c(OCC)c1)C2=O] | 569.3 (M + Na) |
| Example 65 | 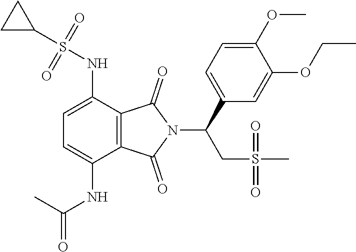 [SMILES: CCOc1cc([C@@H](CS(C)(=O)=O)N2C(=O)c3c(NC(C)=O)ccc(NS(=O)(=O)C4CC4)c3C2=O)ccc1OC] | 580.1 |
| Example 66 | 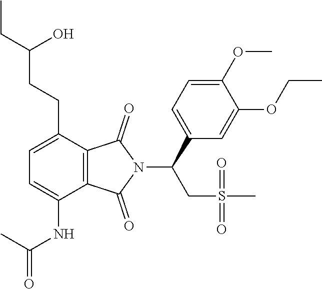 [SMILES: CCOc1cc([C@@H](CS(C)(=O)=O)N2C(=O)c3c(CCC(O)CC)ccc(NC(C)=O)c3C2=O)ccc1OC] | 569.3 (M + Na)+ |
| Example 67 | 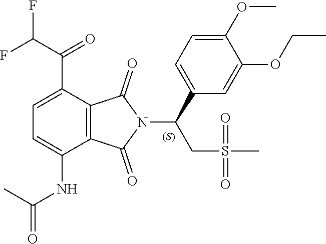 | 539.1 |
| Example 68 | 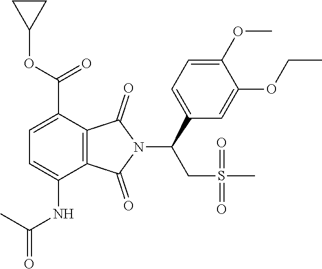 [SMILES: CCOc1cc([C@@H](CS(C)(=O)=O)N2C(=O)c3c(NC(C)=O)ccc(C(=O)OC4CC4)c3C2=O)ccc1OC] | 545.1 |
| Example 69 | 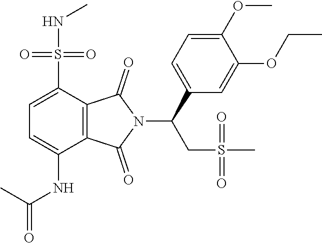 [SMILES: CCOc1cc([C@@H](CS(C)(=O)=O)N2C(=O)c3c(NC(C)=O)ccc(S(=O)(=O)NC)c3C2=O)ccc1OC] | 554.1 |
| Example 70 | 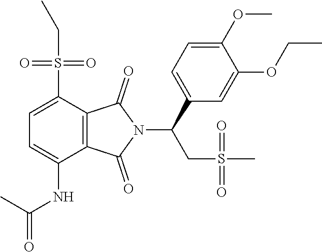 [SMILES: CCOc1cc([C@@H](CS(C)(=O)=O)N2C(=O)c3c(NC(C)=O)ccc(S(=O)(=O)CC)c3C2=O)ccc1OC] | 553.1 |
| Example 71 | 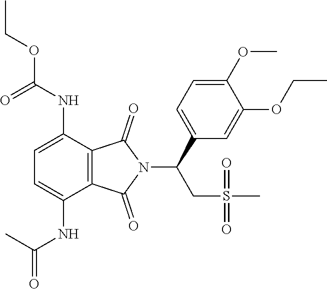 [SMILES: CCOC(=O)Nc1ccc(NC(C)=O)c2c1C(=O)N([C@H](CS(C)(=O)=O)c1ccc(OC)c(OCC)c1)C2=O] | 548.1 |
| Example 72 | 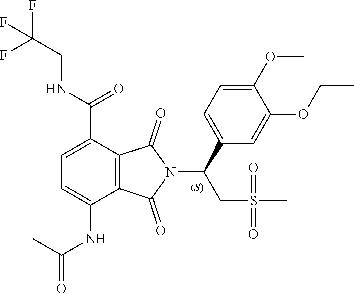 | 586.1 |
| Example 73 | 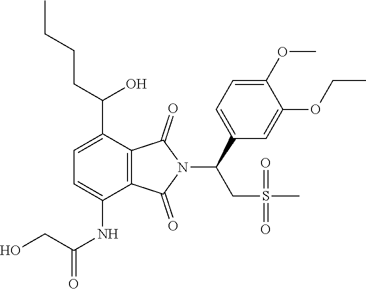 [SMILES: CCCCC(O)c1ccc(NC(=O)CO)c2c1C(=O)N([C@H](CS(C)(=O)=O)c1ccc(OC)c(OCC)c1)C2=O] | 585.2 |
| Example 74 | 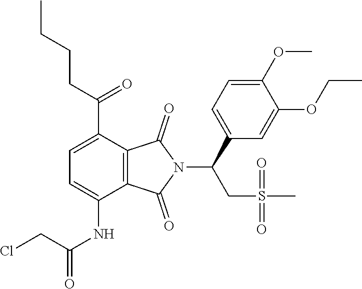 [SMILES: CCCCC(=O)c1ccc(NC(=O)CCl)c2c1C(=O)N([C@H](CS(C)(=O)=O)c1ccc(OC)c(OCC)c1)C2=O] | 579.1 |
| Example 75 | 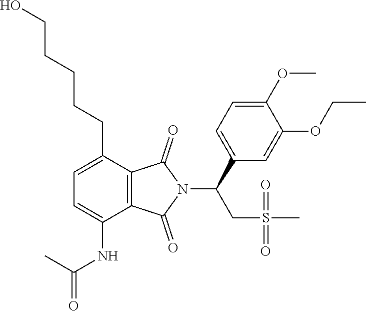 [SMILES: CCOc1cc([C@@H](CS(C)(=O)=O)N2C(=O)c3c(CCCCCO)ccc(NC(C)=O)c3C2=O)ccc1OC] | 569.3 (M + Na)+ |
| Example 76 | 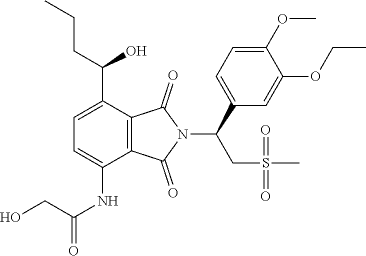 [SMILES: CCC[C@@H](O)c1ccc(NC(=O)CO)c2c1C(=O)N([C@H](CS(C)(=O)=O)c1ccc(OC)c(OCC)c1)C2=O] | 571.2 (M + Na)+ |
| Example 77 | 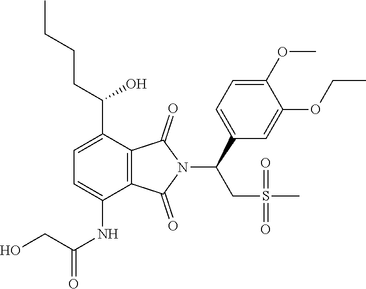 [SMILES: CCCC[C@H](O)c1ccc(NC(=O)CO)c2c1C(=O)N([C@H](CS(C)(=O)=O)c1ccc(OC)c(OCC)c1)C2=O] | 585.2 (M + Na)+ |
| Example 78 | 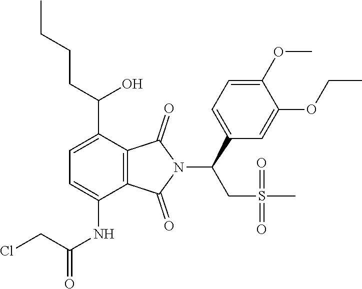 [SMILES: CCCCC(O)c1ccc(NC(=O)CCl)c2c1C(=O)N([C@H](CS(C)(=O)=O)c1ccc(OC)c(OCC)c1)C2=O] | 603.1 (M + Na)+ |
| Example 79 | 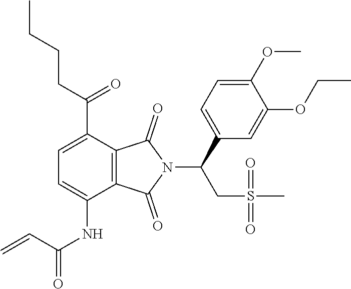 [SMILES: C=CC(=O)Nc1ccc(C(=O)CCCC)c2c1C(=O)N([C@H](CS(C)(=O)=O)c1ccc(OC)c(OCC)c1)C2=O] | 557.1 |
| Example 80 | 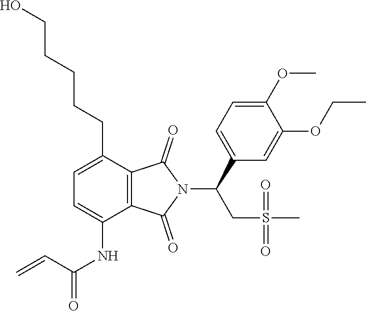 [SMILES: C=CC(=O)Nc1ccc(CCCCCO)c2c1C(=O)N([C@H](CS(C)(=O)=O)c1ccc(OC)c(OCC)c1)C2=O] | 581.2 (M + Na)+ |
| Example 81 | 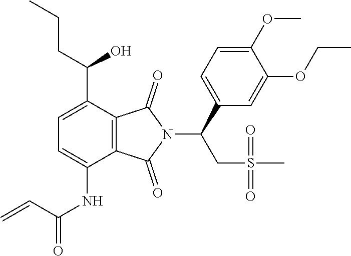 [SMILES: C=CC(=O)Nc1ccc([C@H](O)CCC)c2c1C(=O)N([C@H](CS(C)(=O)=O)c1ccc(OC)c(OCC)c1)C2=O] | 567.1 (M + Na)+ |
| Example 82 | 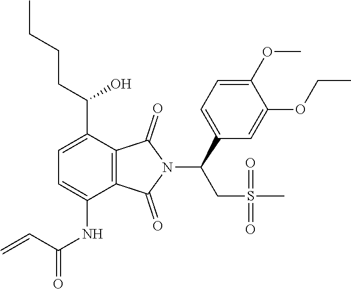 [SMILES: C=CC(=O)Nc1ccc([C@@H](O)CCCC)c2c1C(=O)N([C@H](CS(C)(=O)=O)c1ccc(OC)c(OCC)c1)C2=O] | 581.2 (M + Na)+ |

Examples of biological activity of compounds

1. cLogP value calculation:

Compound cLogP is one of the methods for evaluating the lipophilicity of compounds. A high cLogP value indicates that a compound has stronger lipophilicity, and the compound tends to pass through the lipid layer of the human body more passively (through the principle of compound concentration diffusion). The CLogP values of compounds are shown in the table below:

| Compound No. | CLog P |
|---|---|
| Example 0 | 1.4606 |
| Example 5 | 4.9966 |
| Example 9 | 4.0756 |
| Example 13 | 4.6046 |
| Example 32 | 4.0756 |
| Example 33 | 4.0756 |

Note: Example 0 involved in the present disclosure represents the control drug Apremilast.

2. Inhibition assay of PDE4D3 enzyme:

Materials and Instruments:

PDE4D3 TR-FRET detection kit (BPS, Cat. 60701):
PDE4D3 recombinase
FAM-Cyclic-3′, 5′-AMP
PDE buffer
Tb donor
Binder
Binding buffer A
Binding
buffer B
Black plate (VWR62408-936)
SpectraMax M4 multi-mode reader

Test conditions:

| | Component | Concentration | |
|---|---|---|---|
| Final concentration in | FAM substrate | 100 | nM |
| enzyme reaction step (25 μL) | PDE4D3 | 30 | pg/well |
| | Enzyme reaction | 60 | min |

| | | |
|---|---|---|
| | time | |
| Final concentration in assay | Binder | Dilute at 1:50 |
| reaction (50 μL) | Tb donor | Dilute at 1:1000 |

Reagent preparation:

FAM substrate: A stock solution of 20 μM FAM substrate was diluted to 200 nM with PDE assay buffer. 12.5 μL of the diluted substrate was added to each reaction well.

Compounds: A compound to be tested was first dissolved in DMSO into a 10 mM stock solution. 5 μL of the compound stock solution was added to 45 μL of DMSO to prepare a 1 mM diluent. 5 μL of the 1 mM diluent was added to 45 μL of PDE assay buffer to prepare a gradient dilution starting point. Then, according to the method of adding 5 μL of the previous concentration solution to 15 μL of PDE assay buffer, gradient dilution was carried out for 9 times to prepare compound working solutions with 10 concentrations. The working solutions were added to compound wells at 2.5 μL/well.

PDE4D3: 0.054 μL of a PDE4D3 recombinase stock solution was added to 1500 μL of PDE buffer, and the mixture was added to all compound wells and positive control wells at 10 μL/well. 10 μL of PDE assay buffer was added to substrate control wells.

Binding solution: 3750 μL of binding buffer A and 3750 μL of binding buffer B were mixed well. Then, 150 μL of a binder was added into the mixture and mixed well. 7.5 μL of a Tb donor was added and mixed well. The obtained mixture was added to all wells at 50 μL/well.

| | | | | Sample | Substrate | Tb |
|---|---|---|---|---|---|---|
| Enzyme | Substrate | 12.5 | μL | 12.5 μL | 12.5 μL | 25 μL of |
| reaction | | | | | | assay |
| step | Compound | 2.5 | μL | 2.5 μL of | 2.5 μL of | buffer |
| | | | | assay | assay | |
| | | | | buffer | buffer | |
| | PDE4D3 | 10 | μL | 10 μL of | 10 μL | |
| | | | | assay | | |
| | | | | buffer | | |
| Assay | Binding | 50 | μL | 50 μL | 50 μL | 50 μL |
| reaction | solution | | | | | |

| | |
|---|---|
| step | Encapsulation at room temperature for one hour, |
| | reading 330 nm Excitation, 490 nm, 520 nm Emission |

Data processing:

$$\\ FRET = ( {S}_{520} - ( {Tb}_{520} \times {S}_{490} / {Tb}_{490} ) ) \times 1000 / {S}_{490}$$

S₅₂₀=Read value of sample 520 nm

S₄₉₀=Read value of sample 490 nm

Tb₅₂₀₌Read value of Tb only 520 nm

Tb₄₉₀=Read value of Tb only 490 nm

$$\\ \% Inhibition rate = ( {FRET}_{P} - {FRET}_{S} ) / ( {FRET}_{P} - {FRET}_{Sub} ) \times 100 \%$$

FRET_{S}=Sample FRET

FRETₚ=Positive control FRET

FRET_{sub}=Substrate control FRET.

3. Inhibition assay of PDE4A1 enzyme:

Materials and Instruments:

PDE4A1 detection kit (BPS, Cat. 60340)
PDE4A1 recombinase
FAM-Cyclic-3′, 5′-AMP
PDE buffer
Binder
Binding buffer
Binder diluent (cAMP)
Black plate
Envision 2104 multi-tag reader
PerkinElmer

Test conditions:

| | Component | Concentration | |
|---|---|---|---|
| Enzyme reaction step | FAM substrate | 100 | nM |
| | PDE4A1 | 80 | pg/well |
| | Enzyme reaction | 60 | min |
| | time | | |

| | | |
|---|---|---|
| Assay reaction step | Binder | Dilute at |
| | | 1:100 |

Reagent preparation:

FAM substrate: 25 μL of a FAM substrate stock solution was added into 2500 μL of PDE assay buffer. 25 μL of the diluted substrate was added to each reaction well.

Compounds: A compound to be tested was first dissolved in DMSO into a 10 mM stock solution. 5 μL of the compound stock solution was added to 45 μL of DMSO to prepare a 1 mM diluent. 5 μL of the 1 mM diluent was added to 45 μL of PDE assay buffer to prepare a gradient dilution starting point. Then, according to the method of adding 5 μL of the previous concentration solution to 15 μL of PDE assay buffer, gradient dilution was carried out for 9 times to prepare compound working solutions with 10 concentrations. The working solutions were added to compound wells at 5 μL/well.

PDE4A1: First, a PDE4A1 stock solution was diluted by 100 times to a concentration of 4.9 ng/μL, 1.8 μL of the diluent was added to 2200 μL of PDE assay buffer, and the mixture was added to all compound wells and positive control wells at 20 μL/well. 20 μL of PDE assay buffer was added to substrate control wells.

Binding solution: 95 μL of binding buffer was added into 9.5 mL of binder diluent, and then mixed well. The obtained mixture was added to all wells at 100 μL/well.

| | | Sample | Substrate | Positive |
|---|---|---|---|---|
| Enzyme | Substrate | 25 μL | 25 μL | 25 μL |
| reaction | Compound | 5 μL of | 5 μL of | 5 μL of |
| step | | assay buffer | assay buffer | assay buffer |
| | PDE4A1 | 20 μL of | 20 μL of | 20 μL |
| | | assay buffer | assay buffer | |
| | Encapsulation at room temperature for one hour | | | |
| Assay | Binding | 100 μL | 100 μL | 100 μL |
| reaction | solution | | | |

| | |
|---|---|
| step | Encapsulation at room temperature for 30 min, |
| | reading FP Excitation, 490 nm, 520 nm Emission |

Data processing:

$$Inhibition rate = ( F {P}_{P} - F {P}_{S} ) / ( {FP}_{P} - F {P}_{Sub} ) \times 100 \%$$

FP_{S}=Sample FP

FPₚ=Positive control FP

FP_{sub}=Substrate control FP.

4. Inhibition assay of PDE4B2 enzyme:

Materials and Instruments:

PDE4B2 detection kit (BPS, Cat. 60343)
PDE4B2 recombinase
FAM-Cyclic-3′, 5′-AMP
PDE buffer
Binder
Binding buffer
Binder diluent (cAMP)
Black plate
Envision 2104 multi-tag reader
(PerkinElmer)

Test conditions:

| | Component | Concentration | |
|---|---|---|---|
| Enzyme reaction step | FAM substrate | 100 | nM |
| | PDE4B2 | 150 | pg/well |
| | Enzyme reaction | 60 | min |

| | | |
|---|---|---|
| | time | |
| Assay reaction step | Binder | Dilute at |
| | | 1:100 |

Reagent preparation:

FAM substrate: 25 μL of a FAM substrate stock solution was added into 2500 μL of PDE assay buffer. 25 μL of the diluted substrate was added to each reaction well.

Compounds: A compound to be tested was first dissolved in DMSO into a 10 mM stock solution. 5 μL of the compound stock solution was added to 45 μL of DMSO to prepare a 1 mM diluent. 5 μL of the 1 mM diluent was added to 45 μL of PDE assay buffer to prepare a gradient dilution starting point. Then, according to the method of adding 5 μL of the previous concentration solution to 15 μL of PDE assay buffer, gradient dilution was carried out for 9 times to prepare compound working solutions with 10 concentrations. The working solutions were added to compound wells at 5 μL/well.

PDE4B2: First, a PDE4B2 stock solution was diluted by 100 times to a concentration of 5.2 ng/μL, 3.2 μL of the diluent was added to 2200 μL of PDE assay buffer, and the mixture was added to all compound wells and positive control wells at 20 μL/well. 20 μL of PDE assay buffer was added to substrate control wells.

Binding solution: 95 μL of binder was added into 9.5 mL of binder diluent, and then mixed well. The obtained mixture was added to all wells at 100 μL/well.

| | | Sample | Substrate | Positive |
|---|---|---|---|---|
| Enzyme | Substrate | 25 μL | 25 μL | 25 μL |
| reaction | Compound | 5 μL of | 5 μL of | 5 μL of |
| step | | assay buffer | assay buffer | assay buffer |
| | PDE4B2 | 20 μL of | 20 μL of | 20 μL |
| | | assay buffer | assay buffer | |
| | Encapsulation at room temperature for one hour | | | |
| Assay | Binding | 100 μL | 100 μL | 100 μL |
| reaction | solution | | | |

| | |
|---|---|
| step | Encapsulation at room temperature for one hour, |
| | reading FP Excitation, 490 nm, 520 nm Emission |

Data processing:

$$\% Inhibition rate = ( F {P}_{P} - F {P}_{S} ) / ( {FP}_{P} - F {P}_{Sub} ) \times 100 \%$$

FP_{S}=Sample FP

FPₚ=Positive control FP

FP_{sub}=Substrate control FP.

5. Inhibition assay of PDE4C1 enzyme:

Materials and Instruments:

PDE4C1 detection kit (BPS, Cat. 60384)
PDE4C1 recombinase
FAM-Cyclic-3′, 5′-AMP
PDE buffer
Binder
Binding buffer
Binder diluent (cAMP)
Black plate
Envision 2104 multi-tag reader (PerkinElmer)

Test conditions:

| | Component | Concentration | |
|---|---|---|---|
| Enzyme reaction step | FAM substrate | 100 | nM |
| | PDE4C1 | 400 | pg/well |
| | Enzyme reaction | 60 | min |

| | | |
|---|---|---|
| | time | |
| Assay reaction step | Binder | Dilute at |
| | | 1:100 |

Reagent preparation:

FAM substrate: 12.5 μL of a FAM substrate stock solution was added into 1250 μL of PDE assay buffer. 12.5 μL of the diluted substrate was added to each reaction well.

Compounds: A compound to be tested was first dissolved in DMSO into a 10 mM stock solution. 5 μL of the compound stock solution was added to 45 μL of DMSO to prepare a 1 mM diluent. 5 μL of the 1 mM diluent was added to 45 μL of PDE assay buffer to prepare a gradient dilution starting point. Then, according to the method of adding 5 μL of the previous concentration solution to 15 μL of PDE assay buffer, gradient dilution was carried out for 9 times to prepare compound working solutions with 10 concentrations. The working solutions were added to compound wells at 2.5 μL/well.

PDE4C1: First, a PDE4C1 stock solution was diluted by 100 times to a concentration of 3.2 ng/μL, 13.75 μL of the diluent was added to 1100 μL of PDE assay buffer, and the mixture was added to all compound wells and positive control wells at 10 μL/well. 10 μL of PDE assay buffer was added to substrate control wells.

Binding solution: 50 μL of binder was added into 5 mL of binder diluent, and then mixed well. The obtained mixture was added to all wells at 50 μL/well.

| | | Sample | Substrate | Positive |
|---|---|---|---|---|
| Enzyme | Substrate | 12.5 μL | 12.5 μL | 12.5 μL |
| reaction | Compound | 2.5 μL of | 2.5 μL of | 2.5 μL of |
| step | | assay buffer | assay buffer | assay buffer |
| | PDE4C1 | 10 μL of | 10 μL of | 10 μL |
| | | assay buffer | assay buffer | |
| | Encapsulation at room temperature for one hour | | | |
| Assay | Binding | 50 μL | 50 μL | 50 μL |
| reaction | solution | | | |

| | |
|---|---|
| step | Encapsulation at room temperature for 20 min, |
| | reading FP Excitation, 490 nm, 520 nm Emission |

Data processing:

$$\% Inhibition rate = ( F {P}_{P} - F {P}_{S} ) / ( {FP}_{P} - F {P}_{Sub} ) \times 100 \%$$

FP_{S}=Sample FP

FPₚ=Positive control FP

FP_{sub}=Substrate control FP.

6. The experimental results show that the compound in the examples according to the present disclosure has an inhibitory effect on PDE4, with representative compound examples as follows:

IC₅₀ table of PDE4D3:

| | PDE4D3 |
|---|---|
| Compound No. | (nM) |
| Example 0 | B |
| Example 1 | C |
| Example 2 | B |
| Example 3 | D |
| Example 4 | B |
| Example 5 | B |
| Example 6 | C |
| Example 7 | B |
| Example 8 | C |
| Example 9 | A+ |
| Example 10 | C |
| Example 11 | D |
| Example 12 | D |
| Example 13 | B |
| Example 14 | D |
| Example 15 | D |
| Example 16 | D |
| Example 17 | D |
| Example 18 | C |
| Example 19 | D |
| Example 20 | C |
| Example 21 | D |
| Example 22 | D |
| Example 23 | C |
| Example 24 | B |
| Example 25 | B |
| Example 26 | B |
| Example 27 | B |
| Example 28 | A |
| Example 29 | A |
| Example 30 | C |
| Example 31 | C |
| Example 32 | A+ |
| Example 33 | A+ |
| Example 55 | A+ |
| Example 56 | B |

The above biological activities are A+<5 nM; A=5-10 nM; B=10-50 nM; C=50-200 nM; D>200 nM. The embodiments of the present disclosure have been described, but the present disclosure is not limited to the aforementioned embodiments. Any modification, equivalent replacement, improvement, and the like made within the spirit and principles of the present disclosure shall be included within the scope of protection of the present disclosure.

Note: Example 0 involved in the present disclosure represents the control drug Apremilast. IC₅₀ table of PDE4A1:

| | PDE4A1 |
|---|---|
| Compound No. | (nM) |
| Example 0 | B |
| Example 5 | A |
| Example 9 | A |
| Example 13 | B |
| Example 32 | A |
| Example 33 | A |
| Example 55 | A |
| Example 56 | A |

The above biological activities are A<100 nM; B=100-200 nM. The embodiments of the present disclosure have been described, but the present disclosure is not limited to the aforementioned embodiments. Any modification, equivalent replacement, improvement, and the like made within the spirit and principles of the present disclosure shall be included within the scope of protection of the present disclosure.

Note: Example 0 involved in the present disclosure represents the control drug Apremilast. IC50 table of PDE4B2:

| | PDE4B2 |
|---|---|
| Compound No. | (nM) |
| Example 0 | B |
| Example 5 | A |
| Example 9 | A |
| Example 13 | B |
| Example 32 | A |
| Example 33 | A |
| Example 55 | A |
| Example 56 | A |

The above biological activities are A<100 nM; B=100-200 nM. The embodiments of the present disclosure have been described, but the present disclosure is not limited to the aforementioned embodiments. Any modification, equivalent replacement, improvement, and the like made within the spirit and principles of the present disclosure shall be included within the scope of protection of the present disclosure.

Note: Example 0 involved in the present disclosure represents the control drug Apremilast. IC50 table of PDE4C1:

| | PDE4C1 |
|---|---|
| Compound No. | (nM) |
| Example 0 | B |
| Example 5 | B |
| Example 9 | A |
| Example 13 | B |
| Example 32 | A |
| Example 33 | A |
| Example 55 | A |
| Example 56 | B |

The above biological activities are A<200 nM; B=200-500 nM. The embodiments of the present disclosure have been described, but the present disclosure is not limited to the aforementioned embodiments. Any modification, equivalent replacement, improvement, and the like made within the spirit and principles of the present disclosure shall be included within the scope of protection of the present disclosure.

Note: Example 0 involved in the present disclosure represents the control drug Apremilast.

7. Selective inhibitory activity assay for PDE1, 2, 3, 5, 7, 10 and 11 enzymes

The selective specificity of compounds for PDE4 was assessed by assaying single concentrations of the compounds. For example, PDE1a enzyme, PDElc enzyme, PDE2a enzyme, PDE3a enzyme, PDE3b enzyme, PDE5a1 enzyme, PDE7a enzyme, PDE7b enzyme, PDE10a1 enzyme and PDE11a4 enzyme were tested. The selective inhibitory effects of different compounds on the enzymatic activities of PDEIC, PDE2A, PDE3B, PDE5A1, PDE7A, and PDE10A1 at concentrations of 10 μM and 1 μM, respectively, are shown in the table below:

Inhibition rate (%) of PDE1C

| Compound | 1 μM | 10 μM |
|---|---|---|
| Example 0 | 7.77 | 37.15 |
| Example 5 | 8.26 | 30.84 |
| Example 6 | 0 | 17.97 |
| Example 7 | 6.80 | 26.47 |
| Example 9 | 0.49 | 30.60 |
| Example 13 | 5.34 | 51.72 |
| Example 24 | 3.89 | 34.48 |
| Example 25 | 3.89 | 33.75 |
| Example 27 | 12.14 | 67.53 |
| Example 28 | 1.46 | 24.77 |
| Example 29 | 4.13 | 25.98 |

Inhibition rate (%) of PDE2A

| Compound | 1 μM | 10 μM |
|---|---|---|
| Example 0 | 2.95 | 2.35 |
| Example 5 | 0.83 | 9.01 |
| Example 6 | 0 | 3.41 |
| Example 7 | 0.08 | 2.65 |
| Example 9 | 5.83 | 12.18 |
| Example 13 | 0.08 | 13.85 |
| Example 24 | 0 | 7.79 |
| Example 25 | 20.36 | 13.39 |
| Example 27 | 0 | 8.10 |
| Example 28 | 0 | 5.83 |
| Example 29 | 7.19 | 15.82 |

Inhibition rate (%) of PDE3B

| Compound | 1 μM | 10 μM |
|---|---|---|
| Example 0 | 2.44 | 1.56 |
| Example 5 | 0.06 | −0.69 |
| Example 6 | 0.94 | −0.44 |
| Example 7 | 1.81 | 0.06 |
| Example 9 | 5.44 | 0.81 |
| Example 13 | 3.31 | 0.94 |
| Example 24 | 0 | −0.81 |
| Example 25 | 0 | 0.19 |
| Example 27 | 0 | −1.31 |
| Example 28 | 3.69 | 6.44 |
| Example 29 | 0 | −1.94 |

Inhibition rate (%) of PDE5A1

| Compound | 1 μM | 10 μM |
|---|---|---|
| Example 0 | 3.97 | 2.65 |
| Example 5 | 0.66 | 8.39 |
| Example 6 | 34.22 | 34.00 |
| Example 7 | 0 | −2.87 |
| Example 9 | 1.99 | 5.74 |
| Example 13 | 14.13 | 20.31 |
| Example 24 | 0 | 11.48 |
| Example 25 | 0 | 8.17 |
| Example 27 | 0 | 8.14 |
| Example 28 | 0 | 4.16 |
| Example 29 | 5.72 | 12.99 |

Inhibition rate (%) of PDE7A

| Compound | 1 μM | 10 μM |
|---|---|---|
| Example 0 | 9.41 | 11.07 |
| Example 5 | 0 | 1.94 |
| Example 6 | 0 | 13.83 |
| Example 7 | 3.87 | 15.49 |
| Example 9 | 20.75 | 44.54 |
| Example 13 | 0 | 9.96 |
| Example 24 | 0 | 4.15 |
| Example 25 | 4.98 | 24.62 |
| Example 27 | 2.21 | 14.66 |
| Example 28 | 0.55 | 10.51 |
| Example 29 | 17.43 | 19.92 |

Inhibition rate (%) of PDE10A1

| Compound | 1 μM | 10 μM |
|---|---|---|
| Example 0 | 0 | −2.75 |
| Example 5 | 2.21 | 5.46 |
| Example 6 | 4.84 | −16.18 |
| Example 7 | 0 | 1.30 |
| Example 9 | 0 | 10.09 |
| Example 13 | 16.31 | −26.73 |
| Example 24 | 6.04 | 11.44 |
| Example 25 | 3.02 | 11.10 |
| Example 27 | 11.86 | 1.01 |
| Example 28 | 29.06 | 0.04 |
| Example 29 | 0 | 16.13 |

Note: Example 0 involved in the present disclosure represents the control drug Apremilast.

8. Inhibitory assay of inflammatory factors TNF-α, IL-2, INF-γ

Determination of TNF-α, IL-2 and INF-γ induced by LPS/SEB in human peripheral blood mononuclear cells:

1. The purchased PBMC frozen cells were thawed at 37° C., transferred to RMPI1640 medium, and incubated overnight at 37° C. in an incubator containing 5% of CO₂.

2. The next day, the cells were planked at 2×105 cells/well, with 100 μL per well.

3. Triple-series dilution was performed on compounds to be tested. The final drug concentrations were 3000, 1000, 333.33, 111.11, 37.04, 12.35, 4.12, 1.37, 0.46 nM.

4. LPS stimulus was added to each well at a final concentration of 10 ng/ml.

5. Incubation was carried out overnight at 7° C. in an incubator containing 5% of CO₂.

6. On the third day, the cell culture supernatant was collected, and cytokine assay was performed using an MSD method.

The experimental results show that the compounds in the examples according to the present disclosure have inhibitory effects on inflammatory factors such as TNF-α, IL-2, and IFN-γ. Examples of representative compounds are as follows:

| | | IC₅₀ (nM) for | IC₅₀ (nM) for | IC₅₀ (nM) for |
|---|---|---|---|---|
| Cytokine | Stimulus | Example 0 | Example 9 | Roflumilast |
| TNF-α | LPS | 63.8 | 0.82 | 2.83 |
| IL-2 | SEB | 109.9 | 0.44 | 14.7 |
| INF-r | LPS | 15.4 | 0.43 | 1.4 |
| INF-r | SEB | 77.8 | 1.82 | 10.9 |

The compounds provided by the present disclosure significantly increase the activity of inhibiting the expression of inflammation-related factors.

Note: Example 0 involved in the present disclosure represents the control drug Apremilast.

The embodiments of the present disclosure have been described above. However, the present disclosure is not limited to the aforementioned embodiments. Any modification, equivalent replacement, improvement, and the like made within the spirit and principles of the present disclosure shall be included within the scope of protection of the present disclosure. In particular, optical enantiomers, diastereomers, and stereoisomers of the compounds provided by the present disclosure, as well as mixtures thereof are all within the scope of protection of the present disclosure.

What is claimed is:

1. A compound as shown in formula V and its racemates, stereoisomers, tautomers, isotopic labels, solvates, polymorphs, esters, prodrugs or pharmaceutically acceptable salts: 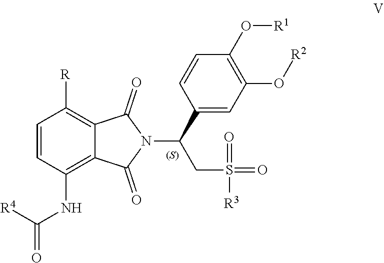
wherein each R refers to the following groups unsubstituted or optionally substituted with one, two or more Ra: hydrocarbyl groups of C4-C10;
Ra is independently selected from deuterium, halogen, amino, hydroxyl, cyano, nitro, oxo;
R¹ refers to the following groups unsubstituted or optionally substituted with one, two or more R1a: hydrocarbyl groups of C1-C16, heteroalkyl groups of C1-C16, and cycloalkyl groups of C3-C12;
R² refers to the following groups unsubstituted or optionally substituted with one, two or more R2a: hydrocarbyl groups of C1-C16, heteroalkyl groups of C1-C16, and cycloalkyl groups of C3-C12;
R³ refers to the following groups unsubstituted or optionally substituted with one, two or more R3a: hydrocarbyl groups of C1-C16, heteroalkyl groups of C1-C16, and cycloalkyl groups of C3-C12;
R⁴ refers to the following groups unsubstituted or optionally substituted with one, two or more R4a: hydrocarbyl groups of C1-C16, heteroalkyl groups of C1-C16, and cycloalkyl groups of C3-C12;
R1a is independently selected from deuterium, halogen, amino, hydroxyl, cyano, nitro, oxo (═O), and the following groups unsubstituted or optionally substituted with one, two or more R1b: hydrocarbyl groups of C1-C16, heteroalkyl groups of C1-C16, and cycloalkyl groups of C3-C12;
R2a is independently selected from deuterium, halogen, amino, hydroxyl, cyano, nitro, oxo (═O), and the following groups unsubstituted or optionally substituted with one, two or more R2b: hydrocarbyl groups of C1-C16, heteroalkyl groups of C1-C16, and cycloalkyl groups of C3-C12;
R3a is independently selected from deuterium, halogen, amino, hydroxyl, cyano, nitro, oxo (═O), and the following groups unsubstituted or optionally substituted with one, two or more R3b: hydrocarbyl groups of C1-C16, heteroalkyl groups of C1-C16, and cycloalkyl groups of C3-C12;
R4a is independently selected from deuterium, halogen, amino, hydroxyl, cyano, nitro, oxo, and the following groups unsubstituted or optionally substituted with one, two or more R4b: hydrocarbyl groups of C1-C16, heteroalkyl groups of C1-C16, and cycloalkyl groups of C3-C12;
R1b is selected from deuterium, halogen, amino, hydroxyl, cyano, nitro, oxo (═O), as well as hydrocarbyl groups of C1-C16, and heteroalkyl groups of C1-C16;
R2b is selected from deuterium, halogen, amino, hydroxyl, cyano, nitro, oxo (═O), as well as hydrocarbyl groups of C1-C16, and heteroalkyl groups of C1-C16;
R3b is selected from deuterium, halogen, amino, hydroxyl, cyano, nitro, oxo (═O), as well as hydrocarbyl groups of C1-C16, and heteroalkyl groups of C1-C16;
R4b is selected from deuterium, halogen, amino, hydroxyl, cyano, nitro, oxo, as well as hydrocarbyl groups of C1-C16, and heteroalkyl groups of C1-C16;
the hydrocarbyl groups are alkanes, alkenes, and alkynes; and the oxo is =O.

2. A compound as shown in formula VI and its racemates, stereoisomers, tautomers, isotopic labels, solvates, polymorphs, esters, prodrugs or pharmaceutically acceptable salts: 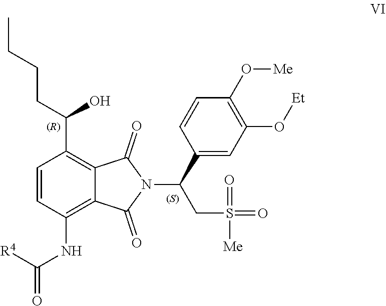
wherein R⁴ refers to the following groups unsubstituted or optionally substituted with one, two or more R4a: hydrocarbyl groups of C1-C16, heteroalkyl groups of C1-C16, and cycloalkyl groups of C3-C12;
R4a is independently selected from deuterium, halogen, amino, hydroxyl, cyano, nitro, oxo, and the following groups unsubstituted or optionally substituted with one, two or more R4b: hydrocarbyl groups of C1-C16, heteroalkyl groups of C1-C16, and cycloalkyl groups of C3-C12; and
R4b is selected from deuterium, halogen, amino, hydroxyl, cyano, nitro, oxo, as well as hydrocarbyl groups of C1-C16, and heteroalkyl groups of C1-C16;
the hydrocarbyl groups are alkanes, alkenes, and alkynes.

3. A pharmaceutical composition, which contains the compound as shown in formula V and its racemates, stereoisomers, tautomers, isotopic labels, solvates, polymorphs, esters, prodrugs or pharmaceutically acceptable salts and pharmaceutically acceptable carriers according to claim 1.

4. The application of the compound as shown in formula V and its racemates, stereoisomers, tautomers, isotopic labels, solvates, polymorphs, esters, prodrugs or pharmaceutically acceptable salts according to claim 1 in the preparation of medicines for inhibiting PDE4 enzyme; and
the medicines for inhibiting the PDE4 enzyme ameliorate the conditions by inhibiting the PDE4, and the conditions include, but are not limited to, asthma, inflammation, chronic or acute obstructive pulmonary disease, chronic or acute pneumonia, enteritis, segmental ileitis, psoriasis, seborrheic dermatitis, stasis dermatitis, palmoplantar abscess, psoriatic arthritis, Behcet's disease or colitis.

5. The application of the compound as shown in formula V and its racemates, stereoisomers, tautomers, isotopic labels, solvates, polymorphs, esters, prodrugs or pharmaceutically acceptable salts according to claim 1 in the preparation of medicines for the regulation of intracellular cAMP level.

6. The application of the compound as shown in formula V and its racemates, stereoisomers, tautomers, isotopic labels, solvates, polymorphs, esters, prodrugs or pharmaceutically acceptable salts according to claim 1 in the preparation of medicines capable of inhibiting the production of TNF-α or NF-κB.

7. The application of the compound as shown in formula V and its racemates, stereoisomers, tautomers, isotopic labels, solvates, polymorphs, esters, prodrugs or pharmaceutically acceptable salts according to claim 1 in the preparation of medicines for the treatment of diseases and conditions selected from the group consisting: depression, asthma, inflammations, chronic or acute obstructive pulmonary disease, chronic or acute pneumonia, viral pneumonia, enteritis, segmental ileitis, Behcet's disease or colitis; and the inflammations comprises contact dermatitis, atopic dermatitis, seborrheic dermatitis, stasis dermatitis, palmoplantar abscess, psoriasis, rheumatoid arthritis, psoriatic arthritis, osteoarthritis, inflammatory dermatosis, or inflammations caused by reperfusion.

8. A method for controlling intracellular cAMP level, comprising the following steps: contacting an effective amount of the compound as shown in formula V and its racemates, stereoisomers, tautomers, isotopic labels, solvates, polymorphs, esters, prodrugs or pharmaceutically acceptable salts according to claim 1 with cells.
